SEQUENCE LISTING (265 sequences)

SEQ ID NO: 1 - DNA, 24 bp, Artificial Sequence
  Synthetic
nannactgatggcgcgagggaggc

SEQ ID NO: 2 - DNA, 19 bp, Artificial Sequence
  Synthetic
gcctccctcgcgccatcag

SEQ ID NO: 3 - DNA, 25 bp, Artificial Sequence
  Synthetic
gccttgccagcccgctcagnnnnnn

SEQ ID NO: 4 - DNA, 19 bp, Artificial Sequence
  Synthetic
ctgagcgggctggcaaggc

SEQ ID NO: 5 - DNA, 15 bp, Artificial Sequence
  Synthetic
gcctccctcgcgcca

SEQ ID NO: 6 - DNA, 15 bp, Artificial Sequence
  Synthetic
gccttgccagcccgc

SEQ ID NO: 7 - DNA, 20 bp, Artificial Sequence
  Synthetic
gctaaggaaagggtgggatg

SEQ ID NO: 8 - DNA, 27 bp, Artificial Sequence
  Synthetic
ttgtgtttgttcataataaaaagtgaa

SEQ ID NO: 9 - DNA, 20 bp, Artificial Sequence
  Synthetic
gagtctccggggctctatgg

SEQ ID NO: 10 - DNA, 20 bp, Artificial Sequence
  Synthetic
gccgctgaagggcttttgaa

SEQ ID NO: 11 - DNA, 20 bp, Artificial Sequence
  Synthetic
ggatcctccccttctttctg

SEQ ID NO: 12 - DNA, 25 bp, Artificial Sequence
  Synthetic
caaaacttgctagttactgcctacc

SEQ ID NO: 13 - DNA, 20 bp, Artificial Sequence
  Synthetic
cccagcacctcttctgagtc

SEQ ID NO: 14 - DNA, 20 bp, Artificial Sequence
  Synthetic
agagaggggtgtaggcatca

SEQ ID NO: 15 - DNA, 20 bp, Artificial Sequence
  Synthetic
ggattgtcaacgtgccctac

SEQ ID NO: 16 - DNA, 20 bp, Artificial Sequence
  Synthetic
gagctagacccggagaggat

SEQ ID NO: 17 - DNA, 20 bp, Artificial Sequence
  Synthetic
gtgcacgaactggtagacga

SEQ ID NO: 18 - DNA, 20 bp, Artificial Sequence
  Synthetic
ggcagaaagcaacacaacct

SEQ ID NO: 19 - DNA, 20 bp, Artificial Sequence
  Synthetic
actgacggcaacagtgaaca

SEQ ID NO: 20 - DNA, 22 bp, Artificial Sequence
  Synthetic
tggaaagtctccctgatgattt

SEQ ID NO: 21 - DNA, 20 bp, Artificial Sequence
  Synthetic
atgcaacaaggttgtgctga

SEQ ID NO: 22 - DNA, 20 bp, Artificial Sequence
  Synthetic
caaacctgaaagaccccagt

SEQ ID NO: 23 - DNA, 20 bp, Artificial Sequence
  Synthetic
agccgactcctaaccgatct

SEQ ID NO: 24 - DNA, 21 bp, Artificial Sequence
  Synthetic
tgaattctgcattttcaccaa

SEQ ID NO: 25 - DNA, 20 bp, Artificial Sequence
  Synthetic
cagagcgagcaaatatggaa

SEQ ID NO: 26 - DNA, 20 bp, Artificial Sequence
  Synthetic
cttgcttcggtttcttgtcc

SEQ ID NO: 27 - DNA, 20 bp, Artificial Sequence
  Synthetic
caaaaacgagacgccaaatc

SEQ ID NO: 28 - DNA, 20 bp, Artificial Sequence
  Synthetic
caaaaacaagacgcgtagca

SEQ ID NO: 29 - DNA, 20 bp, Artificial Sequence
  Synthetic
gaagcggttacaggaatgga

SEQ ID NO: 30 - DNA, 20 bp, Artificial Sequence
  Synthetic
ttctgagctccagcagcttt

SEQ ID NO: 31 - DNA, 20 bp, Artificial Sequence
  Synthetic
gaactgagcagagcagagca

SEQ ID NO: 32 - DNA, 23 bp, Artificial Sequence
  Synthetic
catttggcattaacaaagatcaa

SEQ ID NO: 33 - DNA, 20 bp, Artificial Sequence
  Synthetic
gtgtgacgtggtgaaaggtg

SEQ ID NO: 34 - DNA, 20 bp, Artificial Sequence
  Synthetic
aaatgggcaggagaggaaag

SEQ ID NO: 35 - DNA, 21 bp, Artificial Sequence
  Synthetic
gctaatggtcagaatgctgct

SEQ ID NO: 36 - DNA, 20 bp, Artificial Sequence
  Synthetic
cttcttctgctcctgcgagt

SEQ ID NO: 37 - DNA, 20 bp, Artificial Sequence
  Synthetic
gctgtcaatagtccccaagc

SEQ ID NO: 38 - DNA, 21 bp, Artificial Sequence
  Synthetic
ggatttgcaacctctttatcg

SEQ ID NO: 39 - DNA, 20 bp, Artificial Sequence
  Synthetic
tttggggataagggaaaagg

SEQ ID NO: 40 - DNA, 20 bp, Artificial Sequence
  Synthetic
gctttgctctgtgggctaac

SEQ ID NO: 41 - DNA, 18 bp, Artificial Sequence
  Synthetic
ctgggggacttggcagat

SEQ ID NO: 42 - DNA, 21 bp, Artificial Sequence
  Synthetic
tccaagaaacacagcttctcc

SEQ ID NO: 43 - DNA, 20 bp, Artificial Sequence
  Synthetic
ggctcaggttgtggtagagg

SEQ ID NO: 44 - DNA, 20 bp, Artificial Sequence
  Synthetic
ttgagcctgtcctggaactt

SEQ ID NO: 45 - DNA, 19 bp, Artificial Sequence
  Synthetic
ggagtaggcgcgagctaag

SEQ ID NO: 46 - DNA, 20 bp, Artificial Sequence
  Synthetic
gtccatagtcgctggaggag

SEQ ID NO: 47 - DNA, 18 bp, Artificial Sequence
  Synthetic
cggagtcccaatgaaacg

SEQ ID NO: 48 - DNA, 20 bp, Artificial Sequence
  Synthetic
gaggaggaggacgatgaaga

SEQ ID NO: 49 - DNA, 19 bp, Artificial Sequence
  Synthetic
ccttcccagaagtggtggt

SEQ ID NO: 50 - DNA, 20 bp, Artificial Sequence
  Synthetic
cacacgggagagagacccta

SEQ ID NO: 51 - DNA, 20 bp, Artificial Sequence
  Synthetic
gattcttgggcttcccacat

SEQ ID NO: 52 - DNA, 26 bp, Artificial Sequence
  Synthetic
caaagacacaattagaacagttacca

SEQ ID NO: 53 - DNA, 20 bp, Artificial Sequence
  Synthetic
gcagatcctgccctacacac

SEQ ID NO: 54 - DNA, 20 bp, Artificial Sequence
  Synthetic
agctgaagaaggaactgcca

SEQ ID NO: 55 - DNA, 235 bp, Artificial Sequence
  Synthetic
ggagtaggcgcgagctaagcaggaggcggaggcggaggcggagggcgaggggcggggagcgccgcctggagcgcggcaggaagccttatcagttgtgagtgaggaccagtcgttgtttgagtgtgcctacggaacgccacacctggctaagacagagatgaccgcgtcctcctccagcgactatggacagacttccaagatgagcccacgcgtccctcagcaggattggctgtct

SEQ ID NO: 56 - DNA, 245 bp, Artificial Sequence
  Synthetic
aggtctcaagaatcaaaaacaaaacaaaaatacaaacagagagcaagtgggaagataaataacactccgaaataacctagctacacacttttagtttccaatttttcttagcatgaaatcacttttctcttccatcctgtaagacgtgttctctcctctgcgcatgcactccagggcctgggtgaagacctgcggggccatgccatgctcgtgttgcaggatcaggcactgctccagtgtcaccg

SEQ ID NO: 57 - DNA, 229 bp, Artificial Sequence
  Synthetic
ggggctagcaactctagtatgttttctctcttctgtctattctgggccttcccagaagtggtggtcaggtatcatctcaggtcaagctaccactggaaatgatgatcttccccagcctggaagctccttcttccattactgaaaatgtcttgttcctataggccagaacactcatcacagccatagggtctctctcccgtgtgagttctgtgatgtacaatgagcattg

SEQ ID NO: 58 - DNA, 232 bp, Artificial Sequence
  Synthetic
acgcgggggaagcagcgtgagcagccggaggatcgcggagtcccaatgaaacgggcagccatggccctccacagcccgcagggtgcgtcagggaaatcatgcagccatcaggacacaggctccgggacgtcgagcaccatcctctcctggctgaaaatgacaactatgactcttcatcgtcctcctcctccgaggctgacgtggctgaccgggtctggttcatccgtgacgg

SEQ ID NO: 59 - DNA, 244 bp, Artificial Sequence
  Synthetic
cgattcttgtctcgttccgttttttccttctcaccatctttctgtgtgctgttttcttcattctgatcatggtccccactgtcatcatctttcaaactctcttctgagttgggctgtgaagagctgccctggtctcccggtctgacggtgttgtccaccccatctgaggcacccagggaattgccctggcgtccggagcccgtgggttctgatagcctgggtctttttgcagggaactgatggt

SEQ ID NO: 60 - DNA, 256 bp, Artificial Sequence
  Synthetic
acagagagaacattgtttccatcactcaacaacaaaatgaggaactggctactcaactgcaacaagctctgacagagcgagcaaatatggaattacaacttcaacatgccagagaggcctcccaagtggccaatgaaaaagttcaaaaataaaaattacacacaagaaccaagccccaatgctgatgggcccgcctccaaaaaccggtttattctgctccctcgtcaaaaggacaagaaaccgaagcaaggaataa

SEQ ID NO: 61 - DNA, 149 bp, Artificial Sequence
  Synthetic
gtcactgggtttgccggattcttgggcttcccacatatttcttctttttcttctgatagtgtttcccagattggctccttgatgtgttctggtaactgttctaattgtgtctttgttacttccatggcaaccccttcaggtaagtttca

SEQ ID NO: 62 - DNA, 255 bp, Artificial Sequence
  Synthetic
cgcaaaaaaaagggaggaccactgcgggctctgagcagcaagacttggagcaccgatgacttcttcgcaggactgagggaagagggagaagactccatggctcaggaagaaaaggaggagactggggatgacaatgactgaaggaatgaattgaatcttgagacgggtcctcaccagggtgcctgtggagaaagaatggagtcactgtttaaccatggtacctgcctcagccccagcagaccacaggaggttcgg

SEQ ID NO: 63 - DNA, 248 bp, Artificial Sequence
  Synthetic
gaatcggaagtggctgcgtcgtcgacgctgggctttcgggtcccgcgcccagagatgggctccaaggcaaagaagcgcgtgctgctgccccacccgcccagcgccccccacgggtggagcagatcctggaggatgtgcggggtgcgccggcagaggatccagtgttcaccatcctggccccggaaggctggagtgcagtggcgagatctcgactcactgcaggctccgactccccagttcaagcgatt

SEQ ID NO: 64 - DNA, 233 bp, Artificial Sequence
  Synthetic
ttgggatttttctcttcattatttatcccggagcatttgttgatctgttcaccactcatttgcaacttatatcgccagtccagcagcaaggatattttgtgcagccatggcctccaacgaagatttctccatcacacaagacctggagatcccggcagatattgtggagctccacgacatcaatgtggagccccttcctatggaggacattccgacggaaagcgtccagtacg

SEQ ID NO: 65 - DNA, 101 bp, Artificial Sequence
  Synthetic
ctgggggacttggcagatctcaccgtcaccaacgacaacgacctcagctgcgatgtggagattctggacgcaaagacaagggagaagctgtgtttcttgga

SEQ ID NO: 66 - DNA, 99 bp, Artificial Sequence
  Synthetic
actgacggcaacagtgaacatctcaagcgggagcattcgctcattaagccctaccaagagtgaagatacacaacagcaaatcatcagggagactttcca

SEQ ID NO: 67 - DNA, 101 bp, Artificial Sequence
  Synthetic
gctaatggtcagaatgctgctgggccctctgcagattctgtaactgaaaaaaggcagagtgcttattcactttggaagcgcactcgcaggagcagaagaag

SEQ ID NO: 68 - DNA, 92 bp, Artificial Sequence
  Synthetic
gctgaagaaggaactgccacagggtgatagcactgtccatagcaatgagctgcttctcccggtggtagagggaggccagtgtgtaggggagg

SEQ ID NO: 69 - DNA, 6 bp, Artificial Sequence
  Synthetic
gttcaa

SEQ ID NO: 70 - DNA, 10 bp, Artificial Sequence
  Synthetic
cagagttcaa

SEQ ID NO: 71 - DNA, 11 bp, Artificial Sequence
  Synthetic
gcagagttcaa

SEQ ID NO: 72 - DNA, 18 bp, Artificial Sequence
  Synthetic
gatttaagcagagttcaa

SEQ ID NO: 73 - DNA, 19 bp, Artificial Sequence
  Synthetic
ggatttaagcagagttcaa

SEQ ID NO: 74 - DNA, 20 bp, Artificial Sequence
  Synthetic
tggatttaagcagagttcaa

SEQ ID NO: 75 - DNA, 21 bp, Artificial Sequence
  Synthetic
ctggatttaagcagagttcaa

SEQ ID NO: 76 - DNA, 23 bp, Artificial Sequence
  Synthetic
cactggatttaagcagagttcaa

SEQ ID NO: 77 - DNA, 25 bp, Artificial Sequence
  Synthetic
gccactggatttaagcagagttcaa

SEQ ID NO: 78 - DNA, 27 bp, Artificial Sequence
  Synthetic
cagccactggatttaagcagagttcaa

SEQ ID NO: 79 - DNA, 29 bp, Artificial Sequence
  Synthetic
ctcagccactggatttaagcagagttcaa

SEQ ID NO: 80 - DNA, 30 bp, Artificial Sequence
  Synthetic
actcagccactggatttaagcagagttcaa

SEQ ID NO: 81 - DNA, 30 bp, Artificial Sequence
  Synthetic
aagcccttcagcggccagtagcatctgact

SEQ ID NO: 82 - DNA, 26 bp, Artificial Sequence
  Synthetic
aagcccttcagcggccagtagcatct

SEQ ID NO: 83 - DNA, 25 bp, Artificial Sequence
  Synthetic
aagcccttcagcggccagtagcatc

SEQ ID NO: 84 - DNA, 18 bp, Artificial Sequence
  Synthetic
aagcccttcagcggccag

SEQ ID NO: 85 - DNA, 17 bp, Artificial Sequence
  Synthetic
aagcccttcagcggcca

SEQ ID NO: 86 - DNA, 17 bp, Artificial Sequence
  Synthetic
aagcccttcagcggccg

SEQ ID NO: 87 - DNA, 16 bp, Artificial Sequence
  Synthetic
aagcccttcagcggcc

SEQ ID NO: 88 - DNA, 15 bp, Artificial Sequence
  Synthetic
aagcccttcagcggc

SEQ ID NO: 89 - DNA, 13 bp, Artificial Sequence
  Synthetic
aagcccttcagcg

SEQ ID NO: 90 - DNA, 11 bp, Artificial Sequence
  Synthetic
aagcccttcag

SEQ ID NO: 91 - DNA, 9 bp, Artificial Sequence
  Synthetic
aagcccttc

SEQ ID NO: 92 - DNA, 7 bp, Artificial Sequence
  Synthetic
aagccct

SEQ ID NO: 93 - DNA, 6 bp, Artificial Sequence
  Synthetic
aagccc

SEQ ID NO: 94 - DNA, 29 bp, Artificial Sequence
  Synthetic
gtcagccatggccctccacagcccgcagg

SEQ ID NO: 95 - DNA, 26 bp, Artificial Sequence
  Synthetic
agccagggccctccacagcccgcagg

SEQ ID NO: 96 - DNA, 25 bp, Artificial Sequence
  Synthetic
gccatggccctccacagcccgcagg

SEQ ID NO: 97 - DNA, 18 bp, Artificial Sequence
  Synthetic
ccctccacagcccgcagg

SEQ ID NO: 98 - DNA, 16 bp, Artificial Sequence
  Synthetic
ctccacagcccgcagg

SEQ ID NO: 99 - DNA, 7 bp, Artificial Sequence
  Synthetic
gtgcgtc

SEQ ID NO: 100 - DNA, 10 bp, Artificial Sequence
  Synthetic
gtgcgtcagg

SEQ ID NO: 101 - DNA, 11 bp, Artificial Sequence
  Synthetic
gtgcgtcaggg

SEQ ID NO: 102 - DNA, 18 bp, Artificial Sequence
  Synthetic
gtgcgtcagggaaatcat

SEQ ID NO: 103 - DNA, 20 bp, Artificial Sequence
  Synthetic
gtgcgtcagggaaatcatgc

SEQ ID NO: 104 - DNA, 24 bp, Artificial Sequence
  Synthetic
aggggtgacagtagtagaaagttt

SEQ ID NO: 105 - DNA, 23 bp, Artificial Sequence
  Synthetic
ggggtgacagtagtagaaagttt

SEQ ID NO: 106 - DNA, 22 bp, Artificial Sequence
  Synthetic
gggtgacagtagtagaaagttt

SEQ ID NO: 107 - DNA, 14 bp, Artificial Sequence
  Synthetic
gtagtagaaagttt

SEQ ID NO: 108 - DNA, 11 bp, Artificial Sequence
  Synthetic
gtagaaagttt

SEQ ID NO: 109 - DNA, 9 bp, Artificial Sequence
  Synthetic
agaaagttt

SEQ ID NO: 110 - DNA, 12 bp, Artificial Sequence
  Synthetic
gagagaagactc

SEQ ID NO: 111 - DNA, 13 bp, Artificial Sequence
  Synthetic
gagagaagactca

SEQ ID NO: 112 - DNA, 14 bp, Artificial Sequence
  Synthetic
gagagaagactcaa

SEQ ID NO: 113 - DNA, 22 bp, Artificial Sequence
  Synthetic
gagagaagactcaacccgacac

SEQ ID NO: 114 - DNA, 25 bp, Artificial Sequence
  Synthetic
gagagaagactcaacccgacacttc

SEQ ID NO: 115 - DNA, 27 bp, Artificial Sequence
  Synthetic
gagagaagactcaacccgacacttctc

SEQ ID NO: 116 - DNA, 24 bp, Artificial Sequence
  Synthetic
taggacattctgcacaagagagga

SEQ ID NO: 117 - DNA, 8 bp, Artificial Sequence
  Synthetic
agagagga

SEQ ID NO: 118 - DNA, 7 bp, Artificial Sequence
  Synthetic
gagagga

SEQ ID NO: 119 - DNA, 5 bp, Artificial Sequence
  Synthetic
gagga

SEQ ID NO: 120 - DNA, 12 bp, Artificial Sequence
  Synthetic
gacgcgtacgtg

SEQ ID NO: 121 - DNA, 28 bp, Artificial Sequence
  Synthetic
gacgcgtacgtgaggtcccggacccact

SEQ ID NO: 122 - DNA, 29 bp, Artificial Sequence
  Synthetic
gacgcgtacgtgaggtcccggacccactt

SEQ ID NO: 123 - DNA, 31 bp, Artificial Sequence
  Synthetic
gacgcgtaggtgaggtcccggacccacttct

SEQ ID NO: 124 - DNA, 29 bp, Artificial Sequence
  Synthetic
tcccaagtggccaatgaaaaagttcaaaa

SEQ ID NO: 125 - DNA, 20 bp, Artificial Sequence
  Synthetic
gccaatgaaaaagttcaaaa

SEQ ID NO: 126 - DNA, 14 bp, Artificial Sequence
  Synthetic
gaaaaagttcaaaa

SEQ ID NO: 127 - DNA, 11 bp, Artificial Sequence
  Synthetic
aaagttcaaaa

SEQ ID NO: 128 - DNA, 7 bp, Artificial Sequence
  Synthetic
ataaaaa

SEQ ID NO: 129 - DNA, 16 bp, Artificial Sequence
  Synthetic
ataaaaattacacaca

SEQ ID NO: 130 - DNA, 22 bp, Artificial Sequence
  Synthetic
ataaaaattacacacaagaacc

SEQ ID NO: 131 - DNA, 25 bp, Artificial Sequence
  Synthetic
ataaaaattacacacaagaaccaag

SEQ ID NO: 132 - DNA, 13 bp, Artificial Sequence
  Synthetic
actgcactccagc

SEQ ID NO: 133 - DNA, 13 bp, Artificial Sequence
  Synthetic
actgccctccagc

SEQ ID NO: 134 - DNA, 23 bp, Artificial Sequence
  Synthetic
cttccggggccaggatggtgaac

SEQ ID NO: 135 - DNA, 28 bp, Artificial Sequence
  Synthetic
attgaatcttgagacgggtcctcaccag

SEQ ID NO: 136 - DNA, 21 bp, Artificial Sequence
  Synthetic
cttgagacgggtcctcaccag

SEQ ID NO: 137 - DNA, 16 bp, Artificial Sequence
  Synthetic
gacgggtcctcaccag

SEQ ID NO: 138 - DNA, 12 bp, Artificial Sequence
  Synthetic
ggtcctcaccag

SEQ ID NO: 139 - DNA, 6 bp, Artificial Sequence
  Synthetic
caccag

SEQ ID NO: 140 - DNA, 8 bp, Artificial Sequence
  Synthetic
ggtgcctg

SEQ ID NO: 141 - DNA, 15 bp, Artificial Sequence
  Synthetic
ggtgcctgtggagaa

SEQ ID NO: 142 - DNA, 20 bp, Artificial Sequence
  Synthetic
ggtgcctgtggagaaagaat

SEQ ID NO: 143 - DNA, 24 bp, Artificial Sequence
  Synthetic
ggtgcctgtggagaaagaatggag

SEQ ID NO: 144 - DNA, 30 bp, Artificial Sequence
  Synthetic
ggtgcctgtggagaaagaatggagtcactg

SEQ ID NO: 145 - DNA, 25 bp, Artificial Sequence
  Synthetic
cagcagctaaggatattttgtgcag

SEQ ID NO: 146 - DNA, 11 bp, Artificial Sequence
  Synthetic
ccatggcctcc

SEQ ID NO: 147 - DNA, 32 bp, Artificial Sequence
  Synthetic
actgaaaatgtcttgttcctataggccagaac

SEQ ID NO: 148 - DNA, 31 bp, Artificial Sequence
  Synthetic
ctgaaaatgtcttgttcctataggccagaac

SEQ ID NO: 149 - DNA, 29 bp, Artificial Sequence
  Synthetic
gaaaatgtcttgttcctataggccagaac

SEQ ID NO: 150 - DNA, 27 bp, Artificial Sequence
  Synthetic
aaatgtcttgttcctataggccagaac

SEQ ID NO: 151 - DNA, 24 bp, Artificial Sequence
  Synthetic
tgtcttgttcctataggccagaac

SEQ ID NO: 152 - DNA, 23 bp, Artificial Sequence
  Synthetic
gtcttgttcctataggccagaac

SEQ ID NO: 153 - DNA, 4 bp, Artificial Sequence
  Synthetic
actc

SEQ ID NO: 154 - DNA, 5 bp, Artificial Sequence
  Synthetic
actca

SEQ ID NO: 155 - DNA, 7 bp, Artificial Sequence
  Synthetic
actcatc

SEQ ID NO: 156 - DNA, 9 bp, Artificial Sequence
  Synthetic
actcatcac

SEQ ID NO: 157 - DNA, 12 bp, Artificial Sequence
  Synthetic
actcatcacagc

SEQ ID NO: 158 - DNA, 13 bp, Artificial Sequence
  Synthetic
actcatcacagcc

SEQ ID NO: 159 - DNA, 25 bp, Artificial Sequence
  Synthetic
ggggcgccgcctggagcgcggcagg

SEQ ID NO: 160 - DNA, 22 bp, Artificial Sequence
  Synthetic
gcgccgcctggagcgcggcagg

SEQ ID NO: 161 - DNA, 10 bp, Artificial Sequence
  Synthetic
gcgcggcagg

SEQ ID NO: 162 - DNA, 26 bp, Artificial Sequence
  Synthetic
gggagcgccgcctggagcgcggcagg

SEQ ID NO: 163 - DNA, 25 bp, Artificial Sequence
  Synthetic
ggagcgccgcctggagcgcggcagg

SEQ ID NO: 164 - DNA, 20 bp, Artificial Sequence
  Synthetic
gccgcctggagcgcggcagg

SEQ ID NO: 165 - DNA, 17 bp, Artificial Sequence
  Synthetic
gcctggagcgcggcagg

SEQ ID NO: 166 - DNA, 13 bp, Artificial Sequence
  Synthetic
ggagcgcggcagg

SEQ ID NO: 167 - DNA, 11 bp, Artificial Sequence
  Synthetic
aagccttatca

SEQ ID NO: 168 - DNA, 14 bp, Artificial Sequence
  Synthetic
aagccttatcagtt

SEQ ID NO: 169 - DNA, 26 bp, Artificial Sequence
  Synthetic
aagccttatcagttgtgagtgagaac

SEQ ID NO: 170 - DNA, 10 bp, Artificial Sequence
  Synthetic
aagccttatc

SEQ ID NO: 171 - DNA, 16 bp, Artificial Sequence
  Synthetic
aagccttatcagttgt

SEQ ID NO: 172 - DNA, 19 bp, Artificial Sequence
  Synthetic
aagccttatcagttgtgag

SEQ ID NO: 173 - DNA, 23 bp, Artificial Sequence
  Synthetic
aagccttatcagttgtgagtgag

SEQ ID NO: 174 - DNA, 26 bp, Artificial Sequence
  Synthetic
aagccttatcagttgtgagtgaggac

SEQ ID NO: 175 - DNA, 11 bp, Artificial Sequence
  Synthetic
agaatctccac

SEQ ID NO: 176 - DNA, 12 bp, Artificial Sequence
  Synthetic
cagaatctccac

SEQ ID NO: 177 - DNA, 13 bp, Artificial Sequence
  Synthetic
ccagaatctccac

SEQ ID NO: 178 - DNA, 14 bp, Artificial Sequence
  Synthetic
tccagaatctccac

SEQ ID NO: 179 - DNA, 15 bp, Artificial Sequence
  Synthetic
gtccagaatctccac

SEQ ID NO: 180 - DNA, 18 bp, Artificial Sequence
  Synthetic
tgcgtccagaatctccac

SEQ ID NO: 181 - DNA, 22 bp, Artificial Sequence
  Synthetic
tctttgcgtccagaatctccac

SEQ ID NO: 182 - DNA, 25 bp, Artificial Sequence
  Synthetic
atcgcagctgaggtcgttgtcgttg

SEQ ID NO: 183 - DNA, 24 bp, Artificial Sequence
  Synthetic
atcgcagctgaggtcgttgtcgtt

SEQ ID NO: 184 - DNA, 23 bp, Artificial Sequence
  Synthetic
atcgcagctgaggtcgttgtcgt

SEQ ID NO: 185 - DNA, 22 bp, Artificial Sequence
  Synthetic
atcgcagctgaggtcgttgtcg

SEQ ID NO: 186 - DNA, 21 bp, Artificial Sequence
  Synthetic
atcgcagctgaggtcgttgtc

SEQ ID NO: 187 - DNA, 18 bp, Artificial Sequence
  Synthetic
atcgcagctgaggtcgtt

SEQ ID NO: 188 - DNA, 14 bp, Artificial Sequence
  Synthetic
atcgcagctgaggt

SEQ ID NO: 189 - DNA, 13 bp, Artificial Sequence
  Synthetic
ctgtaactgaaaa

SEQ ID NO: 190 - DNA, 15 bp, Artificial Sequence
  Synthetic
ttctgtaactgaaaa

SEQ ID NO: 191 - DNA, 23 bp, Artificial Sequence
  Synthetic
aaggcagagtgcttattcacttt

SEQ ID NO: 192 - DNA, 16 bp, Artificial Sequence
  Synthetic
gttgtgtatcttcact

SEQ ID NO: 193 - DNA, 18 bp, Artificial Sequence
  Synthetic
ctgttgtgtatcttcact

SEQ ID NO: 194 - DNA, 20 bp, Artificial Sequence
  Synthetic
cttggtagggcttaatgagc

SEQ ID NO: 195 - DNA, 17 bp, Artificial Sequence
  Synthetic
cttggtagggcttaatg

SEQ ID NO: 196 - DNA, 21 bp, Artificial Sequence
  Synthetic
gcactgtccatagcaatgagc

SEQ ID NO: 197 - DNA, 27 bp, Artificial Sequence
  Synthetic
gtgatagcactgtccatagcaatgagc

SEQ ID NO: 198 - DNA, 24 bp, Artificial Sequence
  Synthetic
atagcactgtccatagcaatgagc

SEQ ID NO: 199 - DNA, 16 bp, Artificial Sequence
  Synthetic
gtccatagcaatgagc

SEQ ID NO: 200 - DNA, 10 bp, Artificial Sequence
  Synthetic
agcaatgagc

SEQ ID NO: 201 - DNA, 8 bp, Artificial Sequence
  Synthetic
caatgagc

SEQ ID NO: 202 - DNA, 15 bp, Artificial Sequence
  Synthetic
tgcttctcccggtgg

SEQ ID NO: 203 - DNA, 9 bp, Artificial Sequence
  Synthetic
tgcttctcc

SEQ ID NO: 204 - DNA, 12 bp, Artificial Sequence
  Synthetic
tgcttctcccgg

SEQ ID NO: 205 - DNA, 20 bp, Artificial Sequence
  Synthetic
tgcttctcccggtggtagag

SEQ ID NO: 206 - DNA, 26 bp, Artificial Sequence
  Synthetic
tgcttctcccggtggtagagggaggc

SEQ ID NO: 207 - DNA, 28 bp, Artificial Sequence
  Synthetic
tgcttctcccggtggtagagggaggcca

SEQ ID NO: 208 - DNA, 12 bp, Artificial Sequence
  Synthetic
gagcgcggcagg

SEQ ID NO: 209 - DNA, 16 bp, Artificial Sequence
  Synthetic
cctggagcgcggcagg

SEQ ID NO: 210 - DNA, 19 bp, Artificial Sequence
  Synthetic
ccgcctggagcgcggcagg

SEQ ID NO: 211 - DNA, 23 bp, Artificial Sequence
  Synthetic
agcgccgcctggagcgcggcagg

SEQ ID NO: 212 - DNA, 27 bp, Artificial Sequence
  Synthetic
accccgcgccgcctggagcgcggcagg

SEQ ID NO: 213 - DNA, 43 bp, Artificial Sequence
  Synthetic
atacgagctcttccgatctgagccccgcctggagcgcggcagg

SEQ ID NO: 214 - DNA, 40 bp, Artificial Sequence
  Synthetic
gggggcgaggggcggggagcgccgcctggagcgcggcagg

SEQ ID NO: 215 - DNA, 41 bp, Artificial Sequence
  Synthetic
ggagggcgaggggcggggagcgccgcctggcgcgcggcagg

SEQ ID NO: 216 - DNA, 44 bp, Artificial Sequence
  Synthetic
ccgcccggagttgaaagcgggtgtgaggagcgcggcgcggcagg

SEQ ID NO: 217 - DNA, 46 bp, Artificial Sequence
  Synthetic
gaggcggagggcgaggggcggggagcgccgcctggagcgcggcagg

SEQ ID NO: 218 - DNA, 47 bp, Artificial Sequence
  Synthetic
ggaggcggagggcgaggggcggggagcgccgcctggagcgcggcagg

SEQ ID NO: 219 - DNA, 48 bp, Artificial Sequence
  Synthetic
cggaggcggagggcgaggggcggggagcgccgcctggagcgcggcagg

SEQ ID NO: 220 - DNA, 51 bp, Artificial Sequence
  Synthetic
aagccttatcagttgtgagtgaggaccagtcgttgtttgcgtttgcctacg

SEQ ID NO: 221 - DNA, 86 bp, Artificial Sequence
  Synthetic
gtgtgcaaggctgtccaagcaccagccgccatgcgcgtgtttccgccctggcgaggggctgccagcgccgcctggagcgcggcagg

SEQ ID NO: 222 - DNA, 97 bp, Artificial Sequence
  Synthetic
aagccttatcagttgtgagtgaggaccagtcgttgtttgagtgtgcctacggaacgccacacctggctaagacagagatgaccgcgtcctcctccag

SEQ ID NO: 223 - DNA, 93 bp, Artificial Sequence
  Synthetic
aagccttatcagttgtgagtgaggaccagtcgttgtttgagtgtgcctacggaatgcctcacctgcacaagacagagcagacctcgtccccct

SEQ ID NO: 224 - DNA, 90 bp, Artificial Sequence
  Synthetic
aagccttatcagttgtgagtgaggaccagtcgttgtttgagtgtgcctacggaacgccacacctggctaagacgatcggaagaactcgga

SEQ ID NO: 225 - DNA, 90 bp, Artificial Sequence
  Synthetic
aagccttatcagttgtgagtgaggaccagtcgttgtttgagtgtgccaagatcggaagagctcggatggcgttttctgtttgaaaaaaaa

SEQ ID NO: 226 - DNA, 86 bp, Artificial Sequence
  Synthetic
aagccttatcagttgtgagtgaggaccagtcgttgtttgagtgtgcctacggaacgccacacctggctaagacagggatgaccgca

SEQ ID NO: 227 - DNA, 82 bp, Artificial Sequence
  Synthetic
aagccttatcagttgtgagtgaggaccagtcgttgtttgagtgtgcctacggaacgccacacctggctaagacagagatgac

SEQ ID NO: 228 - DNA, 66 bp, Artificial Sequence
  Synthetic
aagccttatcagttgtgagtgaggaccagtcgttgtttgagtgtgcctacggaacgccacacctgg

SEQ ID NO: 229 - DNA, 69 bp, Artificial Sequence
  Synthetic
aagccttatctgttgtgcgtgagggccagtcgttgtttgagtgtgcctcgggaacgcacacctggctac

SEQ ID NO: 230 - DNA, 68 bp, Artificial Sequence
  Synthetic
aagccttatcagttgtgagtgaggaccagtcgttgtttgagtgtgcttacggaacgccaacacttgct

SEQ ID NO: 231 - DNA, 65 bp, Artificial Sequence
  Synthetic
aagccttatcagttgtgggggaggaccagtcgttgtttgagggtgccttcggaaagccaaccccg

SEQ ID NO: 232 - DNA, 63 bp, Artificial Sequence
  Synthetic
aagccttatcagttgtgagtgaggacccgtcgttgtttgggggggcctacggaagggcaaccc

SEQ ID NO: 233 - DNA, 62 bp, Artificial Sequence
  Synthetic
aagccttatcagttgtgagtgaggaccagagatcggaagagctcggatgccggcttctgctt

SEQ ID NO: 234 - DNA, 62 bp, Artificial Sequence
  Synthetic
aagccttatcagttgtgagtgaggaccagtcgttgtttgggtgggcctcccggacgccaccc

SEQ ID NO: 235 - DNA, 61 bp, Artificial Sequence
  Synthetic
aagccttatcagttgtgagtgaggaccagtcgttgtttggggtgggctacggaacgccaca

SEQ ID NO: 236 - DNA, 58 bp, Artificial Sequence
  Synthetic
gtaccttccgatgtggcggagggctgggggcgaaggccgccgcctagagcgcggcagg

SEQ ID NO: 237 - DNA, 23 bp, Artificial Sequence
  Synthetic
aagccttatcagttgtgcgtcag

SEQ ID NO: 238 - DNA, 19 bp, Artificial Sequence
  Synthetic
tactccttcatggccaccg

SEQ ID NO: 239 - DNA, 20 bp, Artificial Sequence
  Synthetic
agtttgctgacgaagatggc

SEQ ID NO: 240 - DNA, 18 bp, Artificial Sequence
  Synthetic
gccttgcgtgcctatgat

SEQ ID NO: 241 - DNA, 20 bp, Artificial Sequence
  Synthetic
cagtttcctgccagatgtgt

SEQ ID NO: 242 - DNA, 18 bp, Artificial Sequence
  Synthetic
agtgtgggccacctcaag

SEQ ID NO: 243 - DNA, 17 bp, Artificial Sequence
  Synthetic
ggcgtcgagaggaccat

SEQ ID NO: 244 - DNA, 18 bp, Artificial Sequence
  Synthetic
ctgccaccacaacagcag

SEQ ID NO: 245 - DNA, 17 bp, Artificial Sequence
  Synthetic
aagtgactgggcggtgc

SEQ ID NO: 246 - DNA, 18 bp, Artificial Sequence
  Synthetic
agggagctggacctggag

SEQ ID NO: 247 - DNA, 20 bp, Artificial Sequence
  Synthetic
gtcatcccacctgagaacca

SEQ ID NO: 248 - DNA, 22 bp, Artificial Sequence
  Synthetic
cagcctcacttctttgattgaa

SEQ ID NO: 249 - DNA, 20 bp, Artificial Sequence
  Synthetic
gaaaaatggctcggactgtg

SEQ ID NO: 250 - DNA, 20 bp, Artificial Sequence
  Synthetic
gagagctgtggctcctcttc

SEQ ID NO: 251 - DNA, 22 bp, Artificial Sequence
  Synthetic
gttaagggactcaggcagtacg

SEQ ID NO: 252 - DNA, 20 bp, Artificial Sequence
  Synthetic
gggtgctcaaggaaccttct

SEQ ID NO: 253 - DNA, 20 bp, Artificial Sequence
  Synthetic
tcacagagagaccccagacc

SEQ ID NO: 254 - DNA, 19 bp, Artificial Sequence
  Synthetic
catgatgcagtggggacac

SEQ ID NO: 255 - DNA, 20 bp, Artificial Sequence
  Synthetic
cgttgagcctccaggtactc

SEQ ID NO: 256 - DNA, 20 bp, Artificial Sequence
  Synthetic
ccgtaggcacactcaaacaa

SEQ ID NO: 257 - DNA, 20 bp, Artificial Sequence
  Synthetic
cgagagtacctgcacagcat

SEQ ID NO: 258 - DNA, 20 bp, Artificial Sequence
  Synthetic
tcctgtgctttcttcatcca

SEQ ID NO: 259 - DNA, 20 bp, Artificial Sequence
  Synthetic
gtgtaacatgcctccttccg

SEQ ID NO: 260 - DNA, 19 bp, Artificial Sequence
  Synthetic
cggggtgggaaataagcta

SEQ ID NO: 261 - DNA, 20 bp, Artificial Sequence
  Synthetic
gtgctgaaggccaggttgta

SEQ ID NO: 262 - DNA, 20 bp, Artificial Sequence
  Synthetic
gcacttgcttcaggtttcct

SEQ ID NO: 263 - DNA, 23 bp, Artificial Sequence
  Synthetic
tgatcctttagcttggaatgaaa

SEQ ID NO: 264 - DNA, 20 bp, Artificial Sequence
  Synthetic
cctcctcactgccatagagc

SEQ ID NO: 265 - DNA, 23 bp, Artificial Sequence
  Synthetic
gacacagggaagttcaggtagac

(19) United States
(12) Patent Application Publication
(10) Pub. No.: US 2012/0015839 A1
(43) Pub. Date: Jan. 19, 2012

(54) RECURRENT GENE FUSIONS IN CANCER

(75) Inventor: Arul M. Chinnaiyan, Plymouth, MI (US)

(73) Assignee: THE REGENTS OF THE UNIVERSITY OF MICHIGAN, Ann Arbor, MI (US)

(21) Appl. No.: 13/145,067

(22) PCT Filed: Jan. 8, 2010
(86) PCT No.: PCT/US2010/020501

§ 371 Date: Sep. 30, 2011

Related U.S. Application Data
(60) Provisional application No. 61/143,598, filed on Jan. 9, 2009.
(60) Provisional application No. 61/187,776, filed on Jun. 17, 2009.

(51) Int. Cl. C12Q 1/68; C40B 30/04

(52) U.S. Cl. 506/9; 435/6.14; 435/6.11

(57) ABSTRACT

The present invention relates to compositions and methods for cancer diagnosis, research and therapy, including but not limited to, cancer markers. In particular, the present invention relates to recurrent gene fusions as diagnostic markers and clinical targets for cancer (e.g., prostate cancer).

### CROSS REFERENCE TO RELATED APPLICATIONS

This application claims priority to U.S. provisional applications 61/143,598, filed Jan. 9, 2009 and 61/187,776, filed Jun. 17, 2009, each of which is herein incorporated by reference in its entirety.

### GOVERNMENT SUPPORT

This invention was made with government support under grant numbers CA069568, CA 111275 awarded by the National Institutes of Health and grant number W81XWH-08-1-0031 awarded by the Army. The government has certain rights in the invention.

### FIELD OF THE INVENTION

The present invention relates to compositions and methods for cancer diagnosis, research and therapy, including but not limited to, cancer markers. In particular, the present invention relates to recurrent gene fusions as diagnostic markers and clinical targets for cancer (e.g., prostate cancer).

### BACKGROUND OF THE INVENTION

A central aim in cancer research is to identify altered genes that are causally implicated in oncogenesis. Several types of somatic mutations have been identified including base substitutions, insertions, deletions, translocations, and chromosomal gains and losses, all of which result in altered activity of an oncogene or tumor suppressor gene. First hypothesized in the early 1900's, there is now compelling evidence for a causal role for chromosomal rearrangements in cancer (Rowley, Nat Rev Cancer 1: 245 (2001)). Recurrent chromosomal aberrations were thought to be primarily characteristic of leukemias, lymphomas, and sarcomas. Epithelial tumors (carcinomas), which are much more common and contribute to a relatively large fraction of the morbidity and mortality associated with human cancer, comprise less than 1% of the known, disease-specific chromosomal rearrangements (Mitelman, Mutat Res 462: 247 (2000)). While hematological ma lignancies are often characterized by balanced, disease-specific chromosomal rearrangements, most solid tumors have a plethora of non-specific chromosomal aberrations. It is thought that the karyotypic complexity of solid tumors is due to secondary alterations acquired through cancer evolution or progression.

Two primary mechanisms of chromosomal rearrangements have been described. In one mechanism, promoter/enhancer elements of one gene are rearranged adjacent to a proto-oncogene, thus causing altered expression of an oncogenic protein. This type of translocation is exemplified by the apposition of immunoglobulin (IG) and T-cell receptor (TCR) genes to MYC leading to activation of this oncogene in B- and T-cell malignancies, respectively (Rabbitts, Nature 372: 143 (1994)). In the second mechanism, rearrangement results in the fusion of two genes, which produces a fusion protein that may have a new function or altered activity. The prototypic example of this translocation is the BCR-ABL gene fusion in chronic myelogenous leukemia (CML) (Rowley, Nature 243: 290 (1973); de Klein et al., Nature 300: 765 (1982)). Importantly, this finding led to the rational development of imatinib mesylate (Gleevec), which successfully targets the BCR-ABL kinase (Deininger et al., Blood 105: 2640 (2005)). Thus, identifying recurrent gene rearrangements in common epithelial tumors may have profound implications for cancer drug discovery efforts as well as patient treatment.

### SUMMARY OF THE INVENTION

The present invention relates to compositions and methods for cancer diagnosis, research and therapy, including but not limited to, cancer markers. In particular, the present invention relates to recurrent gene fusions as diagnostic markers and clinical targets for cancer (e.g., prostate cancer).

For example, in some embodiments, the present invention provides a method for identifying prostate cancer in a patient comprising: providing a sample from the patient; and detecting the presence or absence in the sample of a gene fusion having a 5′ portion from a transcriptional regulatory region of an SLC45A3 gene and a 3′ portion from an ELK4 gene, wherein detecting the presence in the sample of the gene fusion identifies prostate cancer in the patient. In some embodiments, the transcriptional regulatory region of the SLC45A3 gene comprises a promoter region of the SLC45A3 gene. In some embodiments, the detecting comprises detecting chimeric mRNA transcripts having a 5′ RNA portion transcribed from the transcriptional regulatory region of the SLC45A3 gene and a 3′ RNA portion transcribed from the ELK4 gene. In some embodiments, the gene fusion is a read through transcript. In some embodiments, the sample is tissue, blood, plasma, serum, urine, urine supernatant, urine cell pellet, semen, prostatic secretions or prostate cells. In some embodiments, the method further comprises the step of detecting the presence or absence of a gene fusion having a 5′ portion from a transcriptional regulatory region of an androgen regultated gene or a housekeeping gene and a 3′ portion from an ETS family member gene.

In other embodiments, the present invention provides a method for identifying prostate cancer in a patient comprising: providing a sample from the patient; and detecting the presence or absence in the sample of a gene fusion selected from USP10:ZDHHC7, EIF4E2:HJURP, HJURP:INPP4A,STRN4:GPSN2, RC3H2:RGS3, LMAN2:AP3S1, ZNF649-ZNF577 or MIPOL1:DGKB, wherein detecting the presence in the sample of the gene fusion is identifies prostate cancer in the patient. In some embodiments, the detecting comprises detecting chromosomal rearrangements of genomic DNA. In some embodiments, the detecting comprises detecting chimeric mRNA transcripts or read through transcripts. In some embodiments, the sample is tissue, blood, plasma, serum, urine, urine supernatant, urine cell pellet, semen, prostatic secretions or prostate cells.

In further embodiments, the present invention provides a method for identifying prostate cancer in a patient comprising: providing a sample from the patient; and detecting the presence or absence in the sample of a gene fusion having a 5′ portion from a transcriptional regulatory region of an HERPUD1 gene and a 3′ portion from an ERG gene, wherein detecting the presence in the sample of the gene fusion identifies prostate cancer in the patient.

In yet other embodiments, the present invention provides a method for identifying prostate cancer in a patient comprising: providing a sample from the patient; and detecting the presence or absence in the sample of a gene fusion having a 5′ portion from a transcriptional regulatory region of an AX747630 gene and a 3′ portion from an ETV1 gene, wherein detecting the presence in the sample of the gene fusion identifies prostate cancer in the patient.

In additional embodiments, the present invention provides a method for identifying prostate cancer in a patient comprising: providing a sample from the patient; and detecting the presence or absence in the sample of a gene fusion selected from HERPUD1:ERG, TIA1:DIRC2, NUP214:XKR3, DLEU2:PSPC1, PIK3C2A:TEAD1, SPOCK1:TBC1D9B, or RERE:PIK3CD, wherein detecting the presence in the sample of the gene fusion is identifies prostate cancer in the patient.

Further embodiments of the present invention provide a method for identifying breast cancer in a patient comprising: providing a sample from the patient; and detecting the presence or absence in the sample of a gene fusion selected from AHCYL1:RAD51C, ARHGAP19:DRG1, BC017255:TMEM49, FCHO1:MYO9B, or PAPOLA:AK7, wherein detecting the presence in the sample of the gene fusion is identifies prostate cancer in the patient.

Additional embodiments of the present invention provide a method for identifying prostate cancer in a patient comprising: providing a sample from the patient; and detecting the presence or absence in the sample of a gene fusion selected from the group consisting of SLC45A3-ELK4, ZNF649-ZNF577, CARM1:YIPF2, MGC11102:BANF1, SLC4A1AP:SUPT7L, ERCC2:KLC3, PMF1:BGLAP, THOC6:HCFC1R1, NDUFB8:SEC31L2, ANKRD39:ANKRD23, C14orf124:KIAA0323, C14orf21:CIDEB or ZNF511:TUBGCP2, wherein detecting the presence in the sample of the gene fusion is identifies prostate cancer in the patient

In still further embodiments, the present invention provides a composition comprising at least one of the following: (a) an oligonucleotide probe comprising a sequence that hybridizes to a junction of a chimeric genomic DNA or chimeric mRNA in which a 5′ portion of the chimeric genomic DNA or chimeric mRNA is from a transcriptional regulatory region of an SLC45A3 gene and a 3′ portion of the chimeric genomic DNA or chimeric mRNA is from an ELK4 gene;

(b) a first oligonucleotide probe comprising a sequence that hybridizes to a 5′ portion of a chimeric genomic DNA or chimeric mRNA from a transcriptional regulatory region of an SLC45A3 gene and a second oligonucleotide probe comprising a sequence that hybridizes to a 3′ portion of the chimeric genomic DNA or chimeric mRNA from an ELK4 gene; or

(c) a first amplification oligonucleotide comprising a sequence that hybridizes to a 5′ portion of a chimeric genomic DNA or chimeric mRNA from a transcriptional regulatory region of an SLC45A3 gene and a second amplification oligonucleotide comprising a sequence that hybridizes to a 3′ portion of the chimeric genomic DNA or chimeric mRNA from an ERG gene.

In additional embodiments, the present invention provides a composition comprising at least one of the following:

(a) an oligonucleotide probe comprising a sequence that hybridizes to a junction of a chimeric genomic DNA or chimeric mRNA of a gene fusion selected from the group consisting of USP10:ZDHHC7, EIF4E2:HJURP, HJURP:INPP4A, STRN4:GPSN2, RC3H2:RGS3, LMAN2:AP3S1, ZNF649-ZNF577 and MIPOL1:DGKB;

(b) a first oligonucleotide probe comprising a sequence that hybridizes to a 5′ portion of a chimeric genomic DNA or chimeric mRNA from a gene fusion selected from the group consisting of USP10:ZDHHC7, EIF4E2:HJURP, HJURP:INPP4A, STRN4:GPSN2, RC3H2:RGS3, LMAN2:AP3S1, ZNF649-ZNF577 and MIPOL1:DGKB and a second oligonucleotide probe comprising a sequence that hybridizes to a 3′ portion of the chimeric genomic DNA or chimeric mRNA from a gene fusion selected from the group consisting of USP10:ZDHHC7, EIF4E2:HJURP, HJURP:INPP4A, STRN4:GPSN2, RC3H2:RGS3, LMAN2:AP3S1, ZNF649-ZNF577, MIPOL1:DGKB; or

(c) a first amplification oligonucleotide comprising a sequence that hybridizes to a 5′ portion of a chimeric genomic DNA or chimeric mRNA from a transcriptional regulatory region of an gene fusion selected from the group consisting of USP10:ZDHHC7, EIF4E2:HJURP, HJURP:INPP4A, STRN4:GPSN2, RC3H2:RGS3, LMAN2:AP3S1, ZNF649-ZNF577 and MIPOL1:DGKB and a second amplification oligonucleotide comprising a sequence that hybridizes to a 3′ portion of from a gene fusion selected from the group consisting of USP10:ZDHHC7, EIF4E2:HJURP, HJURP:INPP4A, STRN4:GPSN2, RC3H2:RGS3, LMAN2:AP3S1, ZNF649-ZNF577 and MIPOL1:DGKB.

In some embodiments, the present invention provides a composition comprising at least one of the following:

(a) an oligonucleotide probe comprising a sequence that hybridizes to a junction of a chimeric genomic DNA or chimeric mRNA of a gene fusion selected from HERPUD1:ERG, AX747630:ETV1, TIA1:DIRC2, NUP214:XKR3, DLEU2:PSPC1, PIK3C2A:TEAD1, SPOCK1:TBC1D9B, RERE:PIK3CD, AHCYL1:RAD51C, ARHGAP19:DRG1, BC017255:TMEM49, FCHO1:MYO9B, PAPOLA:AK7, CARM1:YIPF2, MGC11102:BANF1, SLC4A1AP:SUPT7L, ERCC2:KLC3, PMF1:BGLAP, THOC6:HCFC1R1, NDUFB8:SEC31L2, ANKRD39:ANKRD23, C14orf124:KIAA0323, C14orf21:CIDEB, or ZNF511:TUBGCP2;

(b) a first oligonucleotide probe comprising a sequence that hybridizes to a 5′ portion of a chimeric genomic DNA or chimeric mRNA from a gene fusion selected from HERPUD1:ERG, AX747630:ETV1, TIA1:DIRC2, NUP214:XKR3, DLEU2:PSPC1, PIK3C2A:TEAD1, SPOCK1:TBC1D9B, RERE:PIK3CD, AHCYL1:RAD51C, ARHGAP19:DRG1, BC017255:TMEM49, FCHO1:MYO9B, PAPOLA:AK7, CARM1:YIPF2, MGC11102:BANF1, SLC4A1AP:SUPT7L, ERCC2:KLC3, PMF1:BGLAP, THOC6:HCFC1R1, NDUFB8:SEC31L2, ANKRD39:ANKRD23, C14orf124:KIAA0323, C14orf21:CIDEB, or ZNF511:TUBGCP2 and a second oligonucleotide probe comprising a sequence that hybridizes to a 3′ portion of the chimeric genomic DNA or chimeric mRNA from a gene fusion selected from HERPUD1:ERG, AX747630:ETV1, TIA1:DIRC2, NUP214:XKR3, DLEU2:PSPC1, PIK3C2A:TEAD1, SPOCK1:TBC1D9B, RERE:PIK3CD, AHCYL1:RAD51C, ARHGAP19:DRG1, BC017255:TMEM49, FCHO1:MYO9B, PAPOLA:AK7, CARM1:YIPF2, MGC11102:BANF1, SLC4A1AP:SUPT7L, ERCC2:KLC3, PMF1:BGLAP, THOC6:HCFC1R1, NDUFB8:SEC31L2, ANKRD39:ANKRD23, C14orf124:KIAA0323, C14orf21:CIDEB, or ZNF511:TUBGCP2;

(c) a first amplification oligonucleotide comprising a sequence that hybridizes to a 5′ portion of a chimeric genomic DNA or chimeric mRNA from a transcriptional regulatory region of an gene fusion selected from the HERPUD1:ERG, AX747630:ETV1, TIA1:DIRC2, NUP214:XKR3, DLEU2:PSPC1, PIK3C2A:TEAD1, SPOCK1:TBC1D9B, RERE:PIK3CD, AHCYL1:RAD51C, ARHGAP19:DRG1, BC017255:TMEM49, FCHO1:MYO9B, PAPOLA:AK7, CARM1:YIPF2, MGC11102:BANF1, SLC4A1AP:SUPT7L, ERCC2:KLC3, PMF1:BGLAP, THOC6:HCFC1R1, NDUFB8:SEC31L2, ANKRD39:ANKRD23, C14orf124:KIAA0323, C14orf21:CIDEB, or ZNF511:TUBGCP2 and a second amplification oligonucleotide comprising a sequence that hybridizes to a 3′ portion of from a gene fusion selected from HERPUD1:ERG, AX747630:ETV1, TIA1:DIRC2, NUP214:XKR3, DLEU2:PSPC1, PIK3C2A:TEAD1, SPOCK1:TBC1D9B, RERE:PIK3CD, AHCYL1:RAD51C, ARHGAP19:DRG1, BC017255:TMEM49, FCHO1:MYO9B, PAPOLA:AK7, CARM1:YIPF2, MGC11102:BANF1, SLC4A1AP:SUPT7L, ERCC2:KLC3, PMF1:BGLAP, THOC6:HCFC1R1, NDUFB8:SEC31L2, ANKRD39:ANKRD23, C14orf124:KIAA0323, C14orf21:CIDEB, or ZNF511:TUBGCP2.

Additional embodiments of the invention are described herein.

### DESCRIPTION OF THE FIGURES

FIG. 1 shows the “re-discovery” of the BCR-ABL1 gene fusion using massively parallel sequencing of the transcriptome in the chronic myelogenous leukemia cell line K652. The inset represents qRT-PCR validation of the expression of BCR-ABL1 fusion gene in K562 cells.

FIG. 2 shows a schema representing the use of transcriptome sequencing to identify chimeric transcripts. ‘Long read’ sequences compared with the reference database are classified as ‘Mapping’, ‘Partially Aligned’, and ‘Non-Mapping’ reads.

FIG. 3 shows a histogram of predicted VCaP validated chimeras compared to total number of computationally predicted chimeras based on long read technology, short read technology, and an integrative approach.

FIG. 4 shows fusion-chimeras nominated by long read sequences that failed validation by qRT-PCR. TMPRSS2-ERG and USP10-ZDHHC7 were the only two chimeras validated in this set of eighteen candidates in VCaP cells.

FIG. 5 shows representative gene fusions characterized in the prostate cancer cell line VCaP. Top panel, Schematic of USP10-ZDHHC7 fusion on chromosome 16. Exon 1 of USP10 is fused with exon 3 of ZDHHC7, located on the same chromosome in opposite orientation. Inset displays histogram of qRT-PCR validation of USP10-ZDHHC7 transcript. Lower panel, Schematic of a complex intra-chromosomal rearrangement leading to two gene fusions involving HJURP on chromosome 2. Exon 8 of HJURP is fused with exon 2 of EIF4E2 to form HJURP-EIF4E2. Exon 25 of INPP4A is fused with exon 9 of HJURP to form INPP4A-HJURP. Insets display histograms of qRT-PCR validation of HJURP-EIF4E2 and INPP4A-HJURP transcripts.

FIG. 6 shows FISH analysis of the chromosomal rearrangements at 2q11 and 2q37, involving INPP4A, EIF4E2 and HJURP genes. a, Schematic showing genomic organization of INPP4A, EIF4E2 and HJURP genes. Horizontal bars indicate the location of BAC clones. b, FISH analysis using BAC clones 2 and 3 showing the fusion of INPP4A and HJURP genes on a marker chromosome. Arrow indicate the hybridization of 5′ INPP4A probe at 2q11 and 3′HJURP probe at 2q37, respectively, on two copies of normal chromosome 2. c, Hybridization of HJURP probe to two normal copies of chromosome 2 and on the marker chromosome indicate a breakpoint between EIF4E2 and HJURP genes resulting in translocation of 3′ end of chromosome 2q onto the marker chromosome. d, Hybridization of probes 2 and 4 onto two normal chromosome 2, marker chromosome and a split signal on the derivate chromosome 2 (confirming a breakpoint within probes 2 and 4 resulting in an insertion into the marker chromosome. e, Rearrangement of INPP4A gene confirmed by the presence of probe 3 on the marker chromosomes in addition to the co-localizing signal on two copies of normal chromosome 2.

FIG. 7 shows a schematic of MIPOL1-DGKB gene fusion in the prostate cancer cell line LNCaP. MIPOL1-DGKB is an inter-chromosomal gene fusion accompanying the cryptic insertion of ETV1 locus on chromosome 7 into the MIPOL1 intron on chromosome 4. Previously determined genomic breakpoints (stars) are shown in DGKB and MIPOL1 An insertion event results in the inversion of the 3′ end of DGKB and ETV1 into the MIPOL1 intron between exons 10 and 11. Inset displays histogram of qRT-PCR validation of the MIPOL1-DGKB transcript.

FIG. 8 shows FISH analysis of the chromosomal rearrangements involving MIPOL1, DGKB, and ETV1. a, Schematic of the genomic organization of ETV1 and DGKB locus on chromosome 7p21.2. Gene orientation is indicated by arrows. Previously identified genomic breakpoint in DGKB is marked with a star. FISH analysis was performed using BAC clones on VCaP and LNCaP cells. Probe locations encompassing both ETV1 and DGKB are indicated with horizontal bars. Genomic coordinates indicate the region spanning the two BAC clones. b, Co-localized signals (normal) are indicated by arrows and arrowheads indicate the split signal. c, Schematic diagram showing genomic organization of MIPOL1 locus on chromosome 14q13.3-q21.1, d, FISH analysis did not reveal split signals in LNCaP or VCaP cells. e, Genomic organization of MIPOL1, ETV1, and DGKB gene locus on chromosomes 7p21.2 and 14q13.3-q21.1, respectively. f, FISH analysis shows co-localization in LNCaP but not VCaP cells.

FIG. 9 shows chimeric class V, read-through fusions. Schematics of the read-through fusions accompanied with qRT-PCR validations of the fusion transcripts in prostate cancer cell lines VCaP and LNCaP, metastatic prostate tissues VCaP-met and Met 2, and benign prostate cell lines, RWPE and PREC, a, C19orf25-APC2 (intron), b, WDR55-DND1, c, MBTPS2-YY2, and d, ZNF649-ZNF577.

FIG. 10 shows chimera candidates in prostate tissues. a, Schematic of TMPRSS2-ERG fusion boundary populated with short reads sequenced in both VCaP-Met and Met 3 tissues. b, Schematic of the STRN4-GPSN2 fusion on chromosome 19 in the metastatic prostate cancer tissue, Met 3. The 5′ portion of STRN4 is fused with exon 2 of GPSN2, which resides in the opposite orientation on the same chromosome. c, Schematic of RC3H2-RGS3 fusion on chromosome 9 in metastatic prostate cancer tissue, VCaP-Met. The 5′ portion of RC3H2 is fused with exon 20 of RGS3, which resides in the opposite orientation on the same chromosome. d, Schematic of the complex intra chromosomal gene fusion between exon 1 of lectin, mannose-binding 2 (LMAN2) and exon 2 of adaptor-related protein complex 3, subunit 1 (AP3S1). qRT-PCR validation of LMAN2-AP3S1 fusion transcript expression in prostate cancer cell line, VCaP and metastatic prostate tissue, VCaP-Met.

FIG. 11 shows discovery of the recurrent SLC45A3-ELK4 chimera in prostate cancer and a general classification system for chimeric transcripts in cancer. Left upper panel, schematic of the SLC45A3-ELK4 chimera located on chromosome 1. Left middle panel, qRTPCR validation of SLC45A3-ELK4 transcript in a panel of cell lines. Inset, histogram of qRT-PCR assessment of the SLC45A3-ELK4 transcript in LNCaP cells treated with R1881. Left lower panel, histogram of qRT-PCR validation in a panel of prostate tissues benign adjacent prostate, localized prostate cancer (PGA) and metastatic prostate cancer (Mets). Right panel, Chimera classification schema (described below).

FIG. 12 shows lack of rearrangement of the SLC45A3-ELK4 locus in prostate cancers that express the SLC45A3-ELK4 mRNA chimera. Fluorescence in situ hybridization analysis of the ELK4 gene for rearrangement. Schematic diagram (top panel) shows the genomic organization of the SLC45A3 and ELK4 genes on chromosome 1q32.1. BAC clones were derived from the immediately flanking 3′ and 5′ regions of ELK4 and SLC45A3 genes, respectively. Probes were hybridized on the SLC45A3-ELK4 chimera positive cell line LNCaP (a, metaphase spread; b, interphase), and 5 index prostate tumors that express the mRNA chimera (a, e, f, g & h). c, DU145 is a an SLC45A3-ELK4 chimera negative prostate cancer cell line.

FIG. 13 shows genomic level analysis, using Affymetrix SNP 6.0, of 15 samples using the Genotyping Console software. Copy number states are divided into the following categories: 0-homozygous deletion; 1—heterozygous deletion; 2—normal diploid; 3—single copy gain; and 4—multiple copy gain. Genome organization shows the genomic aberrations relative to (a) SLC45A3-ELK4 and (b) PTEN.

FIG. 14 shows a qRT-PCR based survey of a panel of prostate cancer cell lines and tissues—benign, localized prostate cancer, and metastatic tissues for recurrence. USP10-ZDHHC7 (a), INPP4A-HJURP (c), and HJURP-EIF4E2 (d) all show expression in VCaP and VCaP-Met, and were not confirmed in any other samples from the panel. (b) STRN4-GPSN2 expression is confirmed in Met 3.

FIG. 15 shows qRT-PCR based confirmation of fusion transcript expression restricted to prostate cancer samples and absent in somatic tissues from the same patient. Five fusion genes, TMPRSS2-ERG (a), GPSN2-STRN4 (b), USP10-ZDHHC7 (c), RC3H2-RGS3 (d), HJURP-EIF4E2 (e), INPP4A-HJURP (f), LMAN2-AP3S1 (g), MBTPS2-YY2 (h), and ZNF649-ZNF577 (i) were tested in two patients.

FIG. 16 shows FISH analysis of the chromosomal rearrangements involving STRN4-GPSN2 gene fusion in tumor sample MET3. Top panel shows the genomic organization of the GSPN2 and STRN4 genes located on chromosome 19. Normal signal patterns were observed in benign sample (a) whereas a co-localizing signal indicates a gene fusion in tumor sample only (b).

FIG. 17 shows FISH analysis of the chromosomal rearrangements involving EIF4E2-HJURP, USP10-ZDHHC7, and INPP4A-HJURP gene fusions in tumor and paired normal tissues from VCaP-Met. Schematic diagrams on the left panel show the genomic organization of the genes on their respective chromosomes.

FIG. 18 shows FISH analysis of the chromosomal rearrangements involving MRPS10 and HPR. A, Schematic of the MRPS10-HPR fusion. The exons 6-7 of MRPS10 located on chromosome 6 are fused with exon 7 of HPR, on chromosome 16. b, Schematic diagram showing the genomic organization of the HPR gene locus. The horizontal bars indicate the approximate location of the BAC clones from the 5′ and 3′ end of the gene, respectively. c, FISH image from LNCaP cells show two copies of normal chromosome 16, two copies of derivative chromosome 16 [der(16)], and single red signal on derivative chromosome 6 [der(6)] confirming a rearrangement in the HPR gene. d, Schematic diagram showing the genomic organization of the MRPS10 and HPR gene locus. The horizontal bars indicate the approximate location of the BAC clones from the 5′ and 3′ end of MRPS10 and HPR genes, respectively. e, FISH image from LNCaP cells show hybridization of MRPS10 probe to two copies of chromosome 6, and arrows indicate the hybridization of HPR probe to two copies of normal chromosome 16. A single co-localizing signal on der(6) confirms the fusion of MRPS10 with HPR.

FIG. 19 shows a plot of genomic aberrations on chromosome 16 located near the USP10-ZDHHC7 fusion, as seen by array CGH. A deletion involving the two genes is observed in VCaP and the VCaP parental tissue (VCaP-Met), but not in normal prostate cell line, RWPE.

FIG. 20 shows identification of SLC45A3:ELK4 mRNA in urine sediments.

FIG. 21 shows Dynamic range and sensitivity of the paired-end transcriptome analysis relative to single read approaches. (A) Comparison of paired-end and long single transcriptome reads supporting known gene fusions TMPRSS2-ERG, BCR-ABL1, BCAS4-BCAS3, and ARFGEF2-SULF2. (B) Schematic representation of TMPRSS2-ERG in VCaP, comparing mate pairs with long single transcriptome reads. (Upper) Frequency of mate pairs, shown in log scale, are divided based on whether they encompass or span the fusion boundary; (Lower) 100-mer single transcriptome reads spanning TMPRSS2-ERG fusion boundary. (C) Venn diagram of chimera nominations from both a paired-end and long single read strategy for UHR and HBR.

FIG. 22 shows comprehensiveness of paired-end transcriptome analysis. (A) Venn diagram to highlight the overlap between paired-end gene fusion discovery and the previously reported integrated approach applied to VCaP (Left) and LNCaP(Right). Larger circle encompasses all experimentally validated chimeras nominated by paired-end sequencing. The inner circle demonstrates that all previously validated chimeras, previously reported by the integrated approach, are a subset of the paired-end nominations. (B) Histogram of the experimentally validated chimeras in VCaP and K562 highlighting the distinction between known recurrent gene fusions TMPRSS2-ERG and BCR-ABL1 from secondary gene fusions within their respective cell lines. (C) Comprehensive detection of chimeras in MCF-7 using paired-end transcriptome sequencing.

FIG. 23 shows RNA based chimeras. (A) Heatmaps showing the normalized number of reads supporting each readthrough chimera across samples ranging from 0 to 30. (Upper) The heatmap highlights broadly expressed chimeras in UHR, HBR, VCaP, and K562. (Lower) The heatmap highlights the expression of the top ranking restricted gene fusions that are enriched with interchromosomal and intrachromosomal rearrangements. (B) Illustrative examples classifying RNA-based chimeras into (i) read-throughs, (ii) converging transcripts, (iii) diverging transcripts, and (iv) overlapping transcripts. (C Upper) Paired-end approach links reads from independent genes as belonging to the same transcriptional unit (Right), whereas a single read approach would assign these to independent genes (Left). (Lower) The single read approach requires that a chimera span the fusion junction (Left), whereas a paired-end approach can link mate pairs independent of gene annotation (Right).

FIG. 24 shows discovery of previously undescribed ETS gene fusions in localized prostate cancer. (A) Schematic representation of the interchromosomal gene fusion between exon 1 of HERPUD1, residing on chromosome 16, with exon 4 of ERG, located on chromosome 21. (B) Schematic representation showing genomic organization of HERPUD1 and ERG genes. Horizontal bars indicate the location of BAC clones. (Lower) FISH analysis using BAC clones showing HERPUD1 and ERG in a normal tissue (Left), deletion of theERG5_region in tumor (Center), and HERPUD1-ERG fusion in a tumor sample (Right). (C) Schematic representation of the interchromosomal gene fusion between AX747630, residing on chromosome 17, with exon 4 of ETV1 (orange) located on chromosome 21. (D Upper) Schematic representation of the genomic organization of AX747630 and ETV1 genes. (Lower) FISH analysis using BAC clones showing split of ETV1 in tumor sample (Left) and the colocalization of AX747630 and ETV1 in a tumor sample (Right)

FIG. 25 shows paired-end improvements over single-read approach. (A) Paired-end approach resolves ambiguous mappings. (Upper) The single-read approach (Left) displays a single read, or “mate 1,” with identical matches to gene X and gene Y, thus resulting in this read being classified as having multiple mappings. The paired-end approach (Right) displays the same read as the single-read approach aligning to gene X and gene Y. However, the corresponding mate pair, or “mate 2,” aligns with the expected insert size to gene X, but not gene Y. (Lower) Mate 1 shows a best unique hit to gene Y, and a second best hit to gene X, based on single-read approach (Left). However, the second mate, using paired-end (Right), reveals a best unique hit to gene X, revealing the actual best hit. (B) Paired-end sequencing increases coverage spanning fusion junction. Although a single-read approach can detect gene fusions solely by spanning the fusion junction (Left), a paired-end approach can detect a chimera if a mate pairs spans the fusion junction or if the mate pairs encompass the fusion junction (Right), thus providing more opportunity for chimera discovery. (C) Limitation of single-read spanning fusion junction.

FIG. 26 shows paired-end transcriptome sequencing for chimera discovery. (A) Schematic representation of bioinformatics methodology for using paired-end transcriptome sequencing to identify chimeric transcripts. The mate pairs are classified into the following categories (i) mate pairs align to same gene, (ii) mate pairs align to different genes (chimera candidates), (iii) nonmapping, (iv) mitochondrial, (v) ribosomal, and (vi) quality control. The nonmapping mate pairs are further classified based on whether (i) they both fail to map to a gene or (ii) only a single mate read fails to align to a gene. (B) Coverage statistics for UHR and HBR paired-end and long transcriptome read approaches distributed by lane.

FIG. 27 shows novel paired-end schematics and experimental validation. (A) Schematic representation of the UHR paracentric inversion on chromosome 13q34 generating the gene fusion between exon 5 of GAS6 and exon 4 of RASA3. (B) Novel hematological gene fusion NUP214-XKR3. Schematic representation of BCR-ABL1 and NUP214-XKR3 interchromosomal gene fusions between chromosomes 9 and 22. Representative distributions of mate pairs and long single reads areshownonlog scale for both UHR and K562. (C) Histogram of qRT-PCR validation of the NUP214-XKR3 transcript across chronic myeloid leukemia cell lines. (D) Novel complex interchromosomal rearrangement ZDHHC7-ABCB9. Schematic representation of the intrachromosomal rearrangement of USP10-ZDHHC7 and the interchromosomal gene fusion, ZDHHC7-ABCB9. (E) Histogram of qRT-PCR validation of the ZDHHC7-ABCB9 transcript.

FIG. 28 shows validation of novel VCaP interchromosomal gene fusion TIA1-DIRC2. (A) Schematic representation of the VCaP interchromosomal gene fusion between TIA1 residing on chromosome 2 with DIRC2 located on chromosome 3. Inset displays histogram of qRT-PCR validation of the TIA1-DIRC2 transcript. (B) Schematic representation showing genomic organization of TIA1 and DIRC2 genes. Horizontal bars indicate the location of BAC clones (Upper). FISH analysis using BAC clones showing the fusion of TIA1 and DIRC2 genes on a marker chromosome (Lower).

FIG. 29 shows experimental validation of novel chimeras. Quantitative RT-PCR validation of novel paired end nominations (A) ARHGAP19-DRG1, (B) BC017255-TMEM49, (C) AHCYL1-RAD51C, (D) MYO9B-FCHO1, and (E) PAPOLA-AK7 in MCF-7. Validation of prostate tumor chimeras includes (F) HERPUD1-ERG in aT64 and (G) AX747630-ETV1 in aT52. (H) Overall summary of novel validated chimeras.

FIG. 30 shows RNA-Seq gene expression and androgen regulation of HERPUD1 and AX747630 in LNCaP and VCaP androgen time course. Histogram represents the normalized gene expression value of (A) HERPUD1 and (B) AX747630 in LNCaP and VCaP cell lines starved and treated with R1881 at 6, 24, and 48 h. (C) ChIP-Seq binding reveals AR regulation of HERPUD1 and AX747630 in prostate cell lines. Schematic representation of ChIP-Seq peaks representing androgen binding near the upstream of HERPUD1 (Left) and AX747630 (Right) in LNCaP and VCaP.

### DEFINITIONS

To facilitate an understanding of the present invention, a number of terms and phrases are defined below:

As used herein, the term “gene fusion” refers to a chimeric genomic DNA, a chimeric messenger RNA, a truncated protein or a chimeric protein resulting from the fusion of at least a portion of a first gene to at least a portion of a second gene. The gene fusion need not include entire genes or exons of genes.

As used herein, the term “gene upregulated in cancer” refers to a gene that is expressed (e.g., mRNA or protein expression) at a higher level in cancer (e.g., prostate cancer) relative to the level in other tissues. In some embodiments, genes upregulated in cancer are expressed at a level at least 10%, preferably at least 25%, even more preferably at least 50%, still more preferably at least 100%, yet more preferably at least 200%, and most preferably at least 300% higher than the level of expression in other tissues. In some embodiments, genes upregulated in prostate cancer are “androgen regulated genes.”

As used herein, the term “gene upregulated in prostate tissue” refers to a gene that is expressed (e.g., mRNA or protein expression) at a higher level in prostate tissue relative to the level in other tissue. In some embodiments, genes upregulated in prostate tissue are expressed at a level at least 10%, preferably at least 25%, even more preferably at least 50%, still more preferably at least 100%, yet more preferably at least 200%, and most preferably at least 300% higher than the level of expression in other tissues. In some embodiments, genes upregulated in prostate tissue are exclusively expressed in prostate tissue.

As used herein, the term “high expression promoter” refers to a promoter that when fused to a gene causes the gene to be expressed in a particular tissue (e.g., prostate) at a higher level (e.g, at a level at least 10%, preferably at least 25%, even more preferably at least 50%, still more preferably at least 100%, yet more preferably at least 200%, and most preferably at least 300% higher) than the level of expression of the gene when not fused to the high expression promoter. In some embodiments, high expression promoters are promoters from an androgen regulated gene or a housekeeping gene (e.g., HNRPA2B1).

As used herein, the term “transcriptional regulatory region” refers to the region of a gene comprising sequences that modulate (e.g., upregulate or downregulate) expression of the gene. In some embodiments, the transcriptional regulatory region of a gene comprises non-coding upstream sequence of a gene, also called the 5′ untranslated region (5′UTR). In other embodiments, the transcriptional regulatory region contains sequences located within the coding region of a gene or within an intron (e.g., enhancers).

As used herein, the term “androgen regulated gene” refers to a gene or portion of a gene whose expression is induced or repressed by an androgen (e.g., testosterone). The promoter region of an androgen regulated gene may contain an “androgen response element” that interacts with androgens or androgen signaling molecules (e.g., downstream signaling molecules).

As used herein, the terms “detect”, “detecting” or “detection” may describe either the general act of discovering or discerning or the specific observation of a detectably labeled composition.

As used herein, the term “inhibits at least one biological activity of a gene fusion” refers to any agent that decreases any activity of a gene fusion of the present invention (e.g., including, but not limited to, the activities described herein), via directly contacting gene fusion protein, contacting gene fusion mRNA or genomic DNA, causing conformational changes of gene fusion polypeptides, decreasing gene fusion protein levels, or interfering with gene fusion interactions with signaling partners, and affecting the expression of gene fusion target genes. Inhibitors also include molecules that indirectly regulate gene fusion biological activity by intercepting upstream signaling molecules.

As used herein, the term “siRNAs” refers to small interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to, or substantially complementary to, a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

As used herein, the term “stage of cancer” refers to a qualitative or quantitative assessment of the level of advancement of a cancer. Criteria used to determine the stage of a cancer include, but are not limited to, the size of the tumor and the extent of metastases (e.g., localized or distant).

As used herein, the term “gene transfer system” refers to any means of delivering a composition comprising a nucleic acid sequence to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system” refers to gene transfer systems comprising intact or altered viruses belonging to the family Adenoviridae.

As used herein, the term “site-specific recombination target sequences” refers to nucleic acid sequences that provide recognition sequences for recombination factors and the location where recombination takes place.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under ‘medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄ H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄ H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄ H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

As used herein, the term “amplification oligonucleotide” refers to an oligonucleotide that hybridizes to a target nucleic acid, or its complement, and participates in a nucleic acid amplification reaction. An example of an amplification oligonucleotide is a “primer” that hybridizes to a template nucleic acid and contains a 3′ OH end that is extended by a polymerase in an amplification process. Another example of an amplification oligonucleotide is an oligonucleotide that is not extended by a polymerase (e.g., because it has a 3′ blocked end) but participates in or facilitates amplification. Amplification oligonucleotides may optionally include modified nucleotides or analogs, or additional nucleotides that participate in an amplification reaction but are not complementary to or contained in the target nucleic acid. Amplification oligonucleotides may contain a sequence that is not complementary to the target or template sequence. For example, the 5′ region of a primer may include a promoter sequence that is non-complementary to the target nucleic acid (referred to as a “promoter-primer”). Those skilled in the art will understand that an amplification oligonucleotide that functions as a primer may be modified to include a 5′ promoter sequence, and thus function as a promoter-primer. Similarly, a promoter-primer may be modified by removal of, or synthesis without, a promoter sequence and still function as a primer. A 3′ blocked amplification oligonucleotide may provide a promoter sequence and serve as a template for polymerization (referred to as a “promoter-provider”).

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to at least a portion of another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

### DETAILED DESCRIPTION OF THE INVENTION

The present invention is based on the discovery of recurrent gene fusions in cancer (e.g., prostate cancer). The present invention provides diagnostic, research, and therapeutic methods that either directly or indirectly detect or target the gene fusions. The present invention also provides compositions for diagnostic, research, and therapeutic purposes.

Characterization of specific genomic aberrations in cancers has led to the identification of several successful therapeutic targets, such as BCR-ABL1, PDGFR, ERBB2, and EGFR etc (Lynch et al., New Engl. J. Med. 350:2129 [2004]; Slamon et al., New Engl. J. Med. 344:783 [2001]; Demetri et al., New Engl. J. Med. 347:472 [2002]; Druker et al., New Engl. J. Med. 355:2408 [2006]). Therefore, a major goal in cancer research is to identify causal genetic aberrations. Mutations in cancers have been conventionally identified through cytogenetic and molecular techniques (Mitelman et al., Cancer Genome Anatomy Project [2008]), later supplanted with sequencing of specific cancer types (Greenman et al., Nature 446:153 [2007]; Weir et al., Nature 450:893 [2007]; Wood et al., Science 318:1108 [2007]), or candidate genes (Barber et al., New Engl. J. Med. 351:2883 [2004]). Gene fusions resulting from chromosomal rearrangements in cancer are believed to define the most prevalent category of ‘cancer genes’ (Futreal et al., Nat. Revs. 4:177 [2004]). Typically, an aberrant juxtaposition of two genes may encode a fusion protein (e.g., BCR-ABL1), or the regulatory elements of one gene may drive the aberrant expression of an oncogene (e.g., TMPRSS2-ERG). While gene fusions have been widely described in rare hematological malignancies and sarcomas (Mitelman et al., Cancer Genome Anatomy Project [2008]), the recent discovery of recurrent gene fusions in prostate (Lynch et al., New Engl. J. Med. 350:2129 [2004]; Kumar-Sinha et al., Nat. Rev. 8:497 [2008]) and lung cancers (Choi et al. Cancer Res. 68:4971 [2008]; Koivunen et al., Clin. Cancer Res. 14:4275 [2008]; Perner et al., Neoplasia (New York, N.Y.) 10:298 [2008]; Rikova et al., Cell 131:14 [2007]; Soda et al., Nature 448:561 [2007]) points to their role in common solid tumors as well. Considering their prevalence and common characteristics across cancer types, gene fusions may be regarded as a distinct class of ‘mutations’, with a causal role in carcinogenesis, and being strictly confined to cancer cells, they represent ideal diagnostic markers and rational therapeutic targets.

A number of national efforts are underway to comprehensively characterize the genomic alterations in cancer, including The Cancer Genome Atlas Project (TCGA). More recently, high throughput ‘next generation sequencing’ methods have been used for enumeration of genome-wide aberrations in cancers (Campbell et al., Nature Gen. 40:722 [2008]; Parsons et al., Science 321:1807 [2008]). While considerable effort has been vested in discovering base change mutations (and SNPs) in cancers (Weir et al., Nature 450:893 [2007]; Wood et al., Science 318:1108 [2007]; Cheung et al., Nature 409:953 [2001]; Strausberg et al., Trends Genet. 16:103 [2003]), ‘gene-fusions’ have not been systematically investigated thus far. Part of the reason is that solid tumors pick up many non-specific aberrations during tumor evolution, making it difficult to distinguish causal/driver aberrations from secondary/insignificant mutations. The problem of non-specific genetic aberrations is mitigated by sequencing the transcriptome, which restricts the enquiry to ‘expressed sequences’, thus enriching the data for potentially ‘functional’ mutations. The recent gene fusions discovered in prostate and lung cancer were found through transcriptome (Soda et al., Nature 448:561 [2007]; Tomlins et al., Science 310:644 [2005]) and proteome (Rikova et al., Cell 131:14 [2007]) analyses. During experiments conducted during the course of the present invention, massively parallel transcriptome sequencing was employed to discover chimeric transcripts, representing functional gene fusions.

Additional experiments conducted during the course of development of the present invention demonstrated the effectiveness of paired-end massively parallel transcriptome sequencing for fusion gene discovery. By using a paired-end approach, known gene fusions were rediscovered, as well as previously undescribed gene fusions, and it was possible to hone in on causal gene fusions. The ability to detect 12 previously undescribed gene fusions in 4 commonly used cell lines that eluded any previous efforts conveys the superior sensitivity of a paired-end RNA-Seq strategy compared with existing approaches. Also, it demonstrates that it may be possible to unveil previously undescribed chimeric events in previously characterized samples believed to be devoid of any known driver gene fusions. This was exemplified by the discovery of previously undescribed ETS gene fusions in 2 clinically localized prostate tumor samples that lacked known driver gene fusions.

By analyzing the transcriptome at unprecedented depth, numerous gene fusions were revealed, demonstrating the prevalence of a relatively under-represented class of mutations. A major goal is to discover recurrent gene fusions and to distinguish them from secondary, nonspecific chimeras. Although quantifying expression levels is not proof of whether a gene fusion is a driver or passenger, because a low-level gene fusion could still be causative, it still of major significance that a paired-end strategy clearly distinguished known high-level driving gene fusions, such as BCR-ABL1 and TMPRSS2-ERG, from potential lower level passenger chimeras. Overall, these fusions serve as a model for employing a paired-end nomination strategy for prioritizing leads likely to be high-level driving gene fusions, which would subsequently undergo further functional and experimental evaluation.

One of the major advantages of using a transcriptome approach is that it enables the identification of rearrangements that are not detectable at the DNA level. For example, conventional cytogenetic methods would miss gene fusions produced by paracentric inversions, or sub microscopic events, such as GAS6-RASA3. Also, transcriptome sequencing can unveil RNA chimeras, lacking DNA aberrations, as demonstrated by the discovery of a recurrent, prostate specific, read-through of SLC45A3 with ELK4 in prostate cancers. Further classification of RNA based events using paired-end sequencing revealed numerous broadly expressed chimeras between adjacent genes. Although these were not necessarily read-throughs events, because they typically had different orientations, they represent extensions of transcriptional units beyond their annotated boundaries. Unlike single read based approaches, which require chimeras to span exon boundaries of independent genes, it was possible to detect these events using paired-end sequencing.

The comprehensiveness of a paired-end strategy for gene fusion discovery is attributed to the increased coverage provided by sequencing reads from both ends of a fragment, the ability to resolve ambiguous mappings, thus, maximizing the information from the sequences generated, and the lack of reliance on having to span the fusion junction. In comparison, single read approaches using short reads (36 nt) are limited not only by requiring it to span the fusion junction, but with enough sequence on each side to confidently identify the fusion partners. Although long transcriptome reads are highly desirable to provide sequence specificity when aligning to a reference genome, a 454 based approach is limited by the depth of coverage. Therefore, many of the novel paired-end gene fusions, such as TIA1-DIRC2 or ZDHHC7-ABCB9, eluded an integrative transcriptome sequencing approach. However, to circumvent this issue, one of the first long single read (100 nt) runs generated by the Illumina platform was unveiled. Despite offering a deeper coverage of the transcriptome, compared with previous long single read approaches such as expressed sequence tags (ESTs) or 454 long reads, an increased dynamic range by paired-end sequencing was still observed. Also, despite the slightly longer time, it takes to generate 2×50-nt paired-end over 100-nt transcriptome reads, the paired-end data resulted in 3-fold greater nucleotide coverage. Overall, for comparable resources of generating long single reads, paired-end sequencing provides a more comprehensive catalog of gene fusions within a given sample.

Overall, the advantages of employing a paired-end transcriptome strategy for chimera discovery are demonstrated, allowing establishment of a methodology for mining chimeras. It was further possible to extensively catalogue chimeras in a prostate and hematological cancer models. The sensitivity of this approach is of broad impact and significance for revealing novel causative gene fusions in various cancers while revealing additional private gene fusions that may contribute to tumorigenesis or cooperate with driver gene fusions.

### I. Gene Fusions

The present invention identifies recurrent gene fusions indicative of prostate cancer. The gene fusions are the result of a chromosomal rearrangement of 5′ gene fusion partner and a 5′ gene fusion partner. In some embodiments, the gene fusions are fusions of an androgen regulated gene (ARG) or housekeeping gene (HG) and an ETS family member gene. Despite their recurrence, the junction where the 5′ gene fusion partner fuses to the 3′ fusion partner varies. The recurrent gene fusions have use as diagnostic markers and clinical targets for prostate and other (e.g., breast) cancers.

A. Androgen Regulated Genes Genes regulated by androgenic hormones are of critical importance for the normal physiological function of the human prostate gland. They also contribute to the development and progression of prostate carcinoma. Recognized ARGs include, but are not limited to: TMPRSS2; SLC45A3; HERV-K_{—}22q11.23; C15ORF21; FLJ35294; CANT1; PSA; PSMA; KLK2; SNRK; Seladin-1; and, FKBP51 (Paoloni-Giacobino et al., Genomics 44: 309 (1997); Velasco et al., Endocrinology 145(8): 3913 (2004)). Additional ARGs include, but are not limited to, HERPUD1 and GenBank accession number AX747630.

TMPRSS2 (NM_{—}005656) has been demonstrated to be highly expressed in prostate epithelium relative to other normal human tissues (Lin et al., Cancer Research 59: 4180 (1999)). The TMPRSS2 gene is located on chromosome 21. This gene is located at 41,750,797-41,801,948 bp from the pter (51,151 total bp; minus strand orientation). The human TMPRSS2 protein sequence may be found at GenBank accession no. AAC51784 (Swiss Protein accession no. O15393) and the corresponding cDNA at GenBank accession no. U75329 (see also, Paoloni-Giacobino, et al., Genomics 44: 309 (1997)).

SLC45A3, also known as prostein or P501 S, has been shown to be exclusively expressed in normal prostate and prostate cancer at both the transcript and protein level (Kalos et al., Prostate 60, 246-56 (2004); Xu et al., Cancer Res 61, 1563-8 (2001)).

HERV-K_{—}22q11.23, by EST analysis and massively parallel sequencing, was found to be the second most strongly expressed member of the HERV-K family of human endogenous retroviral elements and was most highly expressed in the prostate compared to other normal tissues (Stauffer et al., Cancer Immun 4, 2 (2004)). While androgen regulation of HERV-K elements has not been described, endogenous retroviral elements have been shown to confer androgen responsiveness to the mouse sex-linked protein gene C4A (Stavenhagen et al., Cell 55, 247-54 (1988)). Other HERV-K family members have been shown to be both highly expressed and estrogen-regulated in breast cancer and breast cancer cell lines (Ono et al., J Virol 61, 2059-62 (1987); Patience et al., J Virol 70, 2654-7 (1996); Wang-Johanning et al., Oncogene 22, 1528-35 (2003)), and sequence from a HERV-K3 element on chromosome 19 was fused to FGFR1 in a case of stem cell myeloproliferative disorder with t(8; 19)(p12; q13.3) (Guasch et al., Blood 101, 286-8 (2003)).

C15ORF21, also known as D-PCA-2, was originally isolated based on its exclusive over-expression in normal prostate and prostate cancer (Weigle et al., Int J Cancer 109, 882-92 (2004)).

FLJ35294 was identified as a member of the “full-length long Japan” (FLJ) collection of sequenced human cDNAs (Nat. Genet. 2004 January; 36(1):40-5. Epub 2003 Dec. 21).

CANT1, also known as sSCAN1, is a soluble calcium-activated nucleotidase (Arch Biochem Biophys. 2002 Oct. 1; 406(1):105-15). CANT1 is a 371-amino acid protein. A cleavable signal peptide generates a secreted protein of 333 residues with a predicted core molecular mass of 37,193 Da. Northern analysis identified the transcript in a range of human tissues, including testis, placenta, prostate, and lung. No traditional apyrase-conserved regions or nucleotide-binding domains were identified in this human enzyme, indicating membership in a new family of extracellular nucleotidases.

HERPUD1 (Homocysteine—And Endoplasmic Reticulum Stress-Inducible Protein, Ubiquitin-Like Domain-Containing, 1) is an endoplasmic reticulum (ER) resident protein whose expression is upregulated in response to ER stress. The GenBank accession number for HERPUD1 is NM_{—}014685.

Gene fusions of the present invention may comprise transcriptional regulatory regions of an ARG. The transcriptional regulatory region of an ARG may contain coding or non-coding regions of the ARG, including the promoter region. The promoter region of the ARG may further comprise an androgen response element (ARE) of the ARG. The promoter region for TMPRSS2, in particular, is provided by GenBank accession number AJ276404.

B. Housekeeping Genes

Housekeeping genes are constitutively expressed and are generally ubiquitously expressed in all tissues. These genes encode proteins that provide the basic, essential functions that all cells need to survive. Housekeeping genes are usually expressed at the same level in all cells and tissues, but with some variances, especially during cell growth and organism development. It is unknown exactly how many housekeeping genes human cells have, but most estimates are in the range from 300-500.

Many of the hundreds of housekeeping genes have been identified. The most commonly known gene, GAPDH (glyceraldehyde-3-phosphate dehydrogenase), codes for an enzyme that is vital to the glycolytic pathway. Another important housekeeping gene is albumin, which assists in transporting compounds throughout the body. Several housekeeping genes code for structural proteins that make up the cytoskeleton such as beta-actin and tubulin. Others code for 18S or 28S rRNA subunits of the ribosome. HNRPA2B1 is a member of the ubiquitously expressed heteronuclear ribonuclear proteins. Its promoter has been shown to be unmetheylated and prevents transcriptional silencing of the CMV promoter in transgenes (Williams et al., BMC Biotechnol 5, 17 (2005)). An exemplary listing of housekeeping genes can be found, for example, in Trends in Genetics, 19, 362-365 (2003).

C. ETS Family Member Genes

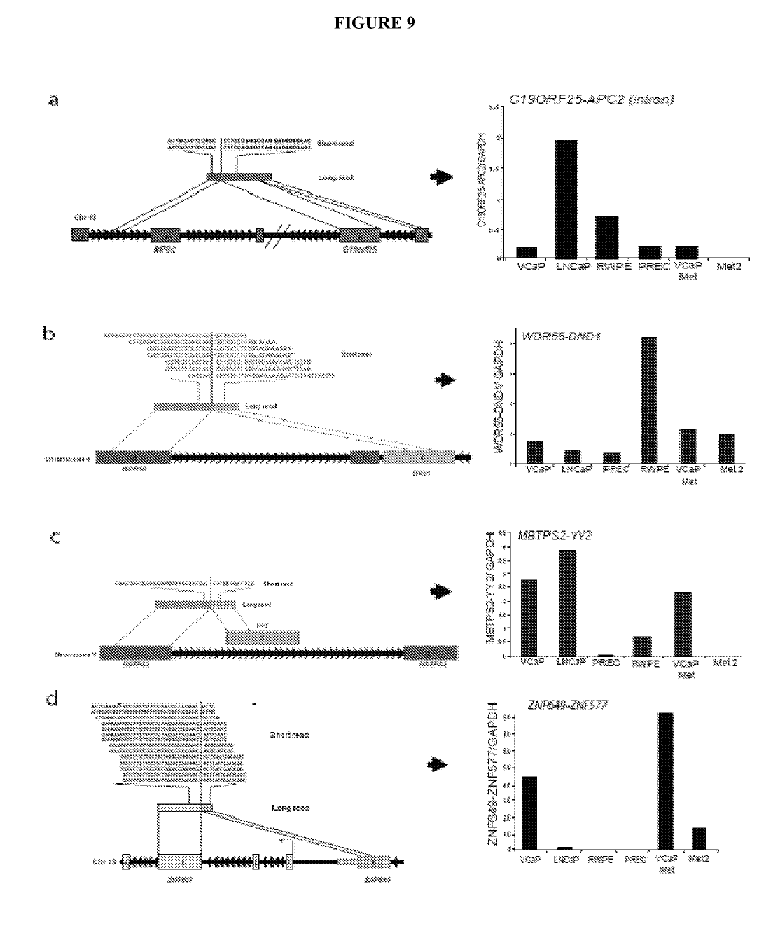

The ETS family of transcription factors regulate the intra-cellular signaling pathways controlling gene expression. As downstream effectors, they activate or repress specific target genes. As upstream effectors, they are responsible for the spacial and temporal expression of numerous growth factor receptors. Almost 30 members of this family have been identified and implicated in a wide range of physiological and pathological processes. These include, but are not limited to: ERG; ETV1 (ER81); FLI1; ETS1; ETS2; ELK1; ETV6 (TEL1); ETV7 (TEL2); GABPα; ELF1; ETV4 (E1AF; PEA3); ETV5 (ERM); ERF; PEA3/E1AF; PU.1; ESE1/ESX; SAP1 (ELK4); ETV3 (METS); EWS/FLI1; ESE1; ESE2 (ELF5); ESE3; PDEF; NET (ELK3; SAP2); NERF (ELF2); and FEV. Exemplary ETS family member sequences are given in FIG. 9.

ERG (NM_{—}004449) has been demonstrated to be highly expressed in prostate epithelium relative to other normal human tissues. The ERG gene is located on chromosome 21. The gene is located at 38,675,671-38,955,488 base pairs from the pter. The ERG gene is 279,817 total by minus strand orientation. The corresponding ERG cDNA and protein sequences are given at GenBank accession nos. M17254 and NP04440 (Swiss Protein acc. no. P11308), respectively.

The ETV1 gene is located on chromosome 7 (GenBank accession nos. NC_{—}000007.11; NC_{—}086703.11; and NT_{—}007819.15). The gene is located at 13,708330-13,803,555 base pairs from the pter. The ETV1 gene is 95,225 bp total, minus strand orientation. The corresponding ETV1 cDNA and protein sequences are given at GenBank accession nos. NM_{—}004956 and NP_{—}004947 (Swiss protein acc. no. P50549), respectively.

The human ETV4 gene is located on chromosome 14 (GenBank accession nos. NC_{—}000017.9; NT_{—}010783.14; and NT_{—}086880.1). The gene is at 38,960,740-38,979,228 base pairs from the pter. The ETV4 gene is 18,488 bp total, minus strand orientation. The corresponding ETV4 cDNA and protein sequences are given at GenBank accession nos. NM_{—}001986 and NP_{—}01977 (Swiss protein acc. no. P43268), respectively.

The human ETV5 gene is located on chromosome 3 at 3q28 (NC_{—}000003.10 (187309570 . . . 187246803). The corresponding ETV5 mRNA and protein sequences are given by GenBank accession nos. NM_{—}004454 and CAG33048, respectively.

D. ETS Gene Fusions

Including the initial identification of TMPRSS2:ETS gene fusions, five classes of ETS rearrangements in prostate cancer have been identified. The present invention is not limited to a particular mechanism. Indeed, an understanding of the mechanism is not necessary to practice the present invention. Nonetheless, it is contemplated that upregulated expression of ETS family members via fusion with an ARG or HG or insertion into a locus with increased expression in cancer provides a mechanism for prostate cancers. Knowledge of the class of rearrangement present in a particular individual allows for customized cancer therapy.

1. Classes of Gene Rearrangements

TMPRSS2:ETS gene fusions (Class I) represent the predominant class of ETS rearrangements in prostate cancer. Rearrangements involving fusions with untranslated regions from other prostate-specific androgen-induced genes (Class IIa) and endogenous retroviral elements (Class IIb), such as SLC45A3 and HERV-K 22q11.23 respectively, function similarly to TMRPSS2 in ETS rearrangements. Similar to the 5′ partners in class I and II rearrangements, C15ORF21 is markedly over-expressed in prostate cancer. However, unlike fusion partners in class I and II rearrangements, C15ORF21 is repressed by androgen, representing a novel class of ETS rearrangements (Class III) involving prostate-specific androgen-repressed 5′ fusion partners. By contrast, HNRPA2B1 did not show prostate-specific expression or androgen-responsiveness. Thus, HNRPA2B1:ETV1 represents a novel class of ETS rearrangements (Class IV) where fusions involving non-tissue specific promoter elements drive ETS expression. In Class V rearrangements, the entire ETS gene is rearranged to prostate-specific regions.

Men with advanced prostate cancer are commonly treated with androgen-deprivation therapy, usually resulting in tumor regression. However the cancer almost invariably progresses with a hormone-refractory phenotype. As Class IV rearrangements (such as HNRPA2B1:ETV1) are driven by androgen insensitive promoter elements, the results indicate that these patients may not respond to anti-androgen treatment, as these gene fusions would not be responsive to androgen-deprivation. Anti-androgen treatment of patients with Class III rearrangements may increase ETS fusion expression. For example, C15ORF21:ETV1 was isolated from a patient with hormone-refractory metastatic prostate cancer where anti-androgen treatment increased C15ORF21:ETV1 expression. Supporting this hypothesis, androgen starvation of LNCaP significantly decreased the expression of endogenous PSA and TMPRSS2, had no effect on HNRPA2B1, and increased the expression of C15ORF21 (FIG. 49). This allows for customized treatment of men with prostate cancer based on the class of fusion present (e.g., the choice of androgen blocking therapy or other alternative therapies).

Multiple classes of gene rearrangements in prostate cancer indicate a more generalized role for chromosomal rearrangements in common epithelial cancers. For example, tissue specific promoter elements may be fused to oncogenes in other hormone driven cancers, such as estrogen response elements fused to oncogenes in breast cancer. Additionally, while prostate specific fusions (Classes I-III, V) would not provide a growth advantage and be selected for in other epithelial cancers, fusions involving strong promoters of ubiquitously expressed genes, such as HNRPA2B1, result in the aberrant expression of oncogenes across tumor types. In summary, this study supports a role for chromosomal rearrangements in common epithelial tumor development through a variety of mechanisms, similar to hematological malignancies.

2. ARG/ETS Gene Fusions

As described above, embodiments of the present invention provide fusions of an ARG to an ETS family member gene. Experiments conducted during the course of development of the present invention indicated that certain fusion genes express fusion transcripts, while others do not express a functional transcript (Tomlins et al., Science, 310: 644-648 (2005); Tomlins et al., Cancer Research 66: 3396-3400 (2006)).

a. ERG Gene Fusions

Gene fusions comprising ERG were found to be the most common gene fusions in prostate cancer. Experiments conducted during the development of embodiments of the present invention identified HERPUD1, an androgen regulated gene, fused to ERG.

b. ETV1 Gene Fusions

Experiments conducted during the development of embodiments of the present invention identified the AX747630:ETV1 fusion. AC747630 has been found to be an androgen regulated gene.

E. Additional Gene Fusions

Embodiments of the present invention provide additional gene fusions associated with prostate cancer, including but not limited to, USP10:ZDHHC7, EIF4E2:HJURP, HJURP-INPP4A,STRN4:GPSN2, RC3H2:RGS3, LMAN2:AP3S1, MIPOL1:DGKB, HERPUD1:ERG, AX747630:ETV1, TIA1:DIRC2, NUP214:XKR3, ZDHHC7:ABCB9, DLEU2:PSPC1, PIK3C2A:TEAD1, SPOCK1:TBC1D9B, and RERE:PIK3CD.

Embodiments of the present invention further provide gene fusions found in additional cancers including, but not limited to, NUP214-XKR3 (chronic myeloid leukemia) and AHCYL1:RAD51C, ARHGAP19:DRG1, BC017255:TMEM49, FCHO1:MYO9B, and PAPOLA:AK7 (breast cancer).

In addition, in some embodiments, the present invention provides gene fusions present or recurrent at the mRNA level but not the DNA level (e.g., read through transcript chimeras). In some embodiments, read through transcripts are the result of cis-splicing. In some embodiments, RNA-based chimeras are categorized as (i) read-throughs, adjacent genes in the same orientation, (ii) diverging genes, adjacent genes in opposite orientation whose 5′ sites are in close proximity, (iii) convergent genes, adjacent genes in opposite orientation whose 3′ ends are in close proximity, and (iv) overlapping genes, adjacent genes who share common exons. Examples of mRNA fusions include, but are not limited to, SLC45A3-ELK4, ZNF649-ZNF577, CARM1:YIPF2, MGC11102:BANF1, SLC4A1AP:SUPT7L, ERCC2:KLC3, PMF1:BGLAP, THOC6:HCFC1R1, NDUFB8:SEC31L2, ANKRD39:ANKRD23, C14orf124:KIAA0323, C14orf21:CIDEB, and ZNF511:TUBGCP2.

F. Multiple Fusions

In some embodiments, samples (e.g., cancer samples) comprise greater than one fusion. For example, experiments conducted during the course of development of the present invention demonstrated that SLC45A3-ELK4 is represented in tumors with other ETS fusions. For example, LNCap cells have ETV1 rearrangement and the SLC45A3-ELK4 fusion. Accordingly, in some embodiments, the present invention provides diagnostic and/or prognostic methods that utilize the detection of multiple fusions in combination.

### II. Antibodies

The gene fusion proteins of the present invention, including fragments, derivatives and analogs thereof, may be used as immunogens to produce antibodies having use in the diagnostic, research, and therapeutic methods described below. The antibodies may be polyclonal or monoclonal, chimeric, humanized, single chain or Fab fragments. Various procedures known to those of ordinary skill in the art may be used for the production and labeling of such antibodies and fragments. See, e.g., Burns, ed., Immunochemical Protocols, 3^{rd} ed., Humana Press (2005); Harlow and Lane, Antibodies: A Laboratory Manual, Cold Spring Harbor Laboratory (1988); Kozbor et al., Immunology Today 4: 72 (1983); Köhler and Milstein, Nature 256: 495 (1975). Antibodies or fragments exploiting the differences between the truncated ETS family member protein or chimeric protein and their respective native proteins are particularly preferred.

### III. Diagnostic Applications

One or more fusions described herein are detectable as DNA, RNA or protein. Initially, the gene fusion is detectable as a chromosomal rearrangement of genomic DNA having a 5′ portion from a 5′ fusion partner and a 3′ portion from a 3′ fusion partner. Once transcribed, the gene fusion is detectable as a chimeric mRNA having a 5′ portion and a 3′ portion. Once translated, the gene fusion is detectable as an amino-terminally truncated 3′ fusion partner or 5′ partner:3′ partner fusion protein. The truncated protein and chimeric protein may differ from their respective native proteins in amino acid sequence, post-translational processing and/or secondary, tertiary or quaternary structure. Such differences, if present, can be used to identify the presence of the gene fusion. Specific methods of detection are described in more detail below.

The present invention provides DNA, RNA and protein based diagnostic methods that either directly or indirectly detect the gene fusions. The present invention also provides compositions and kits for diagnostic purposes.

The diagnostic methods of the present invention may be qualitative or quantitative. Quantitative diagnostic methods may be used, for example, to discriminate between indolent and aggressive cancers via a cutoff or threshold level. Where applicable, qualitative or quantitative diagnostic methods may also include amplification of target, signal or intermediary (e.g., a universal primer).

An initial assay may confirm the presence of a gene fusion but not identify the specific fusion. A secondary assay is then performed to determine the identity of the particular fusion, if desired. The second assay may use a different detection technology than the initial assay.

The gene fusions of the present invention may be detected along with other markers in a multiplex or panel format. Markers are selected for their predictive value alone or in combination with the gene fusions. Exemplary prostate cancer markers include, but are not limited to: AMACR/P504S (U.S. Pat. No. 6,262,245); PCA3 (U.S. Pat. No. 7,008,765); PCGEM1 (U.S. Pat. No. 6,828,429); prostein/P501S, P503S, P504S, P509S, P510S, prostase/P703P, P710P (U.S. Publication No. 20030185830); and, those disclosed in U.S. Pat. Nos. 5,854,206 and 6,034,218, and U.S. Publication No. 20030175736, each of which is herein incorporated by reference in its entirety. Markers for other cancers, diseases, infections, and metabolic conditions are also contemplated for inclusion in a multiplex of panel format.

The diagnostic methods of the present invention may also be modified with reference to data correlating particular gene fusions with the stage, aggressiveness or progression of the disease or the presence or risk of metastasis. Ultimately, the information provided by the methods of the present invention will assist a physician in choosing the best course of treatment for a particular patient.

A. Sample

Any patient sample suspected of containing the gene fusions may be tested according to the methods of the present invention. By way of non-limiting examples, the sample may be tissue (e.g., a prostate biopsy sample or a tissue sample obtained by prostatectomy), blood, urine, semen, prostatic secretions or a fraction thereof (e.g., plasma, serum, urine supernatant, urine cell pellet or prostate cells). A urine sample is preferably collected immediately following an attentive digital rectal examination (DRE), which causes prostate cells from the prostate gland to shed into the urinary tract.

The patient sample typically requires preliminary processing designed to isolate or enrich the sample for the gene fusions or cells that contain the gene fusions. A variety of techniques known to those of ordinary skill in the art may be used for this purpose, including but not limited: centrifugation; immunocapture; cell lysis; and, nucleic acid target capture (See, e.g., EP Pat. No. 1 409 727, herein incorporated by reference in its entirety).

B. DNA and RNA Detection

The gene fusions of the present invention may be detected as chromosomal rearrangements of genomic DNA or chimeric mRNA using a variety of nucleic acid techniques known to those of ordinary skill in the art, including but not limited to: nucleic acid sequencing; nucleic acid hybridization; and, nucleic acid amplification.

1. Sequencing

Illustrative non-limiting examples of nucleic acid sequencing techniques include, but are not limited to, chain terminator (Sanger) sequencing and dye terminator sequencing. Those of ordinary skill in the art will recognize that because RNA is less stable in the cell and more prone to nuclease attack experimentally RNA is usually reverse transcribed to DNA before sequencing.

Chain terminator sequencing uses sequence-specific termination of a DNA synthesis reaction using modified nucleotide substrates. Extension is initiated at a specific site on the template DNA by using a short radioactive, or other labeled, oligonucleotide primer complementary to the template at that region. The oligonucleotide primer is extended using a DNA polymerase, standard four deoxynucleotide bases, and a low concentration of one chain terminating nucleotide, most commonly a di-deoxynucleotide. This reaction is repeated in four separate tubes with each of the bases taking turns as the di-deoxynucleotide. Limited incorporation of the chain terminating nucleotide by the DNA polymerase results in a series of related DNA fragments that are terminated only at positions where that particular di-deoxynucleotide is used. For each reaction tube, the fragments are size-separated by electrophoresis in a slab polyacrylamide gel or a capillary tube filled with a viscous polymer. The sequence is determined by reading which lane produces a visualized mark from the labeled primer as you scan from the top of the gel to the bottom.

Dye terminator sequencing alternatively labels the terminators. Complete sequencing can be performed in a single reaction by labeling each of the di-deoxynucleotide chain-terminators with a separate fluorescent dye, which fluoresces at a different wavelength.

2. Hybridization

Illustrative non-limiting examples of nucleic acid hybridization techniques include, but are not limited to, in situ hybridization (ISH), microarray, and Southern or Northern blot.

In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. RNA ISH is used to measure and localize mRNAs and other transcripts within tissue sections or whole mounts. Sample cells and tissues are usually treated to fix the target transcripts in place and to increase access of the probe. The probe hybridizes to the target sequence at elevated temperature, and then the excess probe is washed away. The probe that was labeled with either radio-, fluorescent- or antigen-labeled bases is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

a. FISH

In some embodiments, fusion sequences are detected using fluorescence in situ hybridization (FISH). The preferred FISH assays for the present invention utilize bacterial artificial chromosomes (BACs). These have been used extensively in the human genome sequencing project (see Nature 409: 953-958 (2001)) and clones containing specific BACs are available through distributors that can be located through many sources, e.g., NCBI. Each BAC clone from the human genome has been given a reference name that unambiguously identifies it. These names can be used to find a corresponding GenBank sequence and to order copies of the clone from a distributor.

The present invention further provides a method of performing a FISH assay on human prostate cells, human prostate tissue or on the fluid surrounding said human prostate cells or human prostate tissue.

Probes are labeled with appropriate fluorescent or other markers and then used in hybridizations. The Examples section provided herein sets forth one particular protocol that is effective for measuring deletions but one of skill in the art will recognize that many variations of this assay can be used equally well. Specific protocols are well known in the art and can be readily adapted for the present invention. Guidance regarding methodology may be obtained from many references including: In situ Hybridization: Medical Applications (eds. G. R. Coulton and J. de Belleroche), Kluwer Academic Publishers, Boston (1992); In situ Hybridization: In Neurobiology; Advances in Methodology (eds. J. H. Eberwine, K. L. Valentino, and J. D. Barchas), Oxford University Press Inc., England (1994); In situ Hybridization: A Practical Approach (ed. D. G. Wilkinson), Oxford University Press Inc., England (1992)); Kuo, et al., Am. J. Hum. Genet. 49:112-119 (1991); Klinger, et al., Am. J. Hum. Genet. 51:55-65 (1992); and Ward, et al., Am. J. Hum. Genet. 52:854-865 (1993)). There are also kits that are commercially available and that provide protocols for performing FISH assays (available from e.g., Oncor, Inc., Gaithersburg, Md.). Patents providing guidance on methodology include U.S. Pat. Nos. 5,225,326; 5,545,524; 6,121,489 and 6,573,043. All of these references are hereby incorporated by reference in their entirety and may be used along with similar references in the art and with the information provided in the Examples section herein to establish procedural steps convenient for a particular laboratory.

b. Microarrays

Different kinds of biological assays are called microarrays including, but not limited to: DNA microarrays (e.g., cDNA microarrays and oligonucleotide microarrays); protein microarrays; tissue microarrays; transfection or cell microarrays; chemical compound microarrays; and, antibody microarrays. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes by comparing gene expression in disease and normal cells. Microarrays can be fabricated using a variety of technologies, including but not limiting: printing with fine-pointed pins onto glass slides; photolithography using pre-made masks; photolithography using dynamic micromirror devices; ink-jet printing; or, electrochemistry on microelectrode arrays.

Southern and Northern blotting is used to detect specific DNA or RNA sequences, respectively. DNA or RNA extracted from a sample is fragmented, electrophoretically separated on a matrix gel, and transferred to a membrane filter. The filter bound DNA or RNA is subject to hybridization with a labeled probe complementary to the sequence of interest. Hybridized probe bound to the filter is detected. A variant of the procedure is the reverse Northern blot, in which the substrate nucleic acid that is affixed to the membrane is a collection of isolated DNA fragments and the probe is RNA extracted from a tissue and labeled.

3. Amplification

Chromosomal rearrangements of genomic DNA and chimeric mRNA may be amplified prior to or simultaneous with detection. Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

The polymerase chain reaction (U.S. Pat. Nos. 4,683,195, 4,683,202, 4,800,159 and 4,965,188, each of which is herein incorporated by reference in its entirety), commonly referred to as PCR, uses multiple cycles of denaturation, annealing of primer pairs to opposite strands, and primer extension to exponentially increase copy numbers of a target nucleic acid sequence. In a variation called RT-PCR, reverse transcriptase (RT) is used to make a complementary DNA (cDNA) from mRNA, and the cDNA is then amplified by PCR to produce multiple copies of DNA. For other various permutations of PCR see, e.g., U.S. Pat. Nos. 4,683,195, 4,683,202 and 4,800,159; Mullis et al., Meth. Enzymol. 155: 335 (1987); and, Murakawa et al., DNA 7: 287 (1988), each of which is herein incorporated by reference in its entirety.

Transcription mediated amplification (U.S. Pat. Nos. 5,480,784 and 5,399,491, each of which is herein incorporated by reference in its entirety), commonly referred to as TMA, synthesizes multiple copies of a target nucleic acid sequence autocatalytically under conditions of substantially constant temperature, ionic strength, and pH in which multiple RNA copies of the target sequence autocatalytically generate additional copies. See, e.g., U.S. Pat. Nos. 5,399,491 and 5,824,518, each of which is herein incorporated by reference in its entirety. In a variation described in U.S. Publ. No. 20060046265 (herein incorporated by reference in its entirety), TMA optionally incorporates the use of blocking moieties, terminating moieties, and other modifying moieties to improve TMA process sensitivity and accuracy.

The ligase chain reaction (Weiss, R., Science 254: 1292 (1991), herein incorporated by reference in its entirety), commonly referred to as LCR, uses two sets of complementary DNA oligonucleotides that hybridize to adjacent regions of the target nucleic acid. The DNA oligonucleotides are covalently linked by a DNA ligase in repeated cycles of thermal denaturation, hybridization and ligation to produce a detectable double-stranded ligated oligonucleotide product.

Strand displacement amplification (Walker, G. et al., Proc. Natl. Acad. Sci. USA 89: 392-396 (1992); U.S. Pat. Nos. 5,270,184 and 5,455,166, each of which is herein incorporated by reference in its entirety), commonly referred to as SDA, uses cycles of annealing pairs of primer sequences to opposite strands of a target sequence, primer extension in the presence of a dNTPαS to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product. Thermophilic SDA (tSDA) uses thermophilic endonucleases and polymerases at higher temperatures in essentially the same method (EP Pat. No. 0 684 315).

Other amplification methods include, for example: nucleic acid sequence based amplification (U.S. Pat. No. 5,130,238, herein incorporated by reference in its entirety), commonly referred to as NASBA; one that uses an RNA replicase to amplify the probe molecule itself (Lizardi et al., BioTechnol. 6: 1197 (1988), herein incorporated by reference in its entirety), commonly referred to as Qβ replicase; a transcription based amplification method (Kwoh et al., Proc. Natl. Acad. Sci. USA 86:1173 (1989)); and, self-sustained sequence replication (Guatelli et al., Proc. Natl. Acad. Sci. USA 87: 1874 (1990), each of which is herein incorporated by reference in its entirety). For further discussion of known amplification methods see Persing, David H., “In Vitro Nucleic Acid Amplification Techniques” in Diagnostic Medical Microbiology: Principles and Applications (Persing et al., Eds.), pp. 51-87 (American Society for Microbiology, Washington, D.C. (1993)).

4. Detection Methods

Non-amplified or amplified gene fusion nucleic acids can be detected by any conventional means. For example, the gene fusions can be detected by hybridization with a detectably labeled probe and measurement of the resulting hybrids. Illustrative non-limiting examples of detection methods are described below.

One illustrative detection method, the Hybridization Protection Assay (HPA) involves hybridizing a chemiluminescent oligonucleotide probe (e.g., an acridinium ester-labeled (AE) probe) to the target sequence, selectively hydrolyzing the chemiluminescent label present on unhybridized probe, and measuring the chemiluminescence produced from the remaining probe in a luminometer. See, e.g., U.S. Pat. No. 5,283,174 and Norman C. Nelson et al., Nonisotopic Probing, Blotting, and Sequencing, ch. 17 (Larry J. Kricka ed., 2d ed. 1995, each of which is herein incorporated by reference in its entirety).

Another illustrative detection method provides for quantitative evaluation of the amplification process in real-time. Evaluation of an amplification process in “real-time” involves determining the amount of amplicon in the reaction mixture either continuously or periodically during the amplification reaction, and using the determined values to calculate the amount of target sequence initially present in the sample. A variety of methods for determining the amount of initial target sequence present in a sample based on real-time amplification are well known in the art. These include methods disclosed in U.S. Pat. Nos. 6,303,305 and 6,541,205, each of which is herein incorporated by reference in its entirety. Another method for determining the quantity of target sequence initially present in a sample, but which is not based on a real-time amplification, is disclosed in U.S. Pat. No. 5,710,029, herein incorporated by reference in its entirety.

Amplification products may be detected in real-time through the use of various self-hybridizing probes, most of which have a stem-loop structure. Such self-hybridizing probes are labeled so that they emit differently detectable signals, depending on whether the probes are in a self-hybridized state or an altered state through hybridization to a target sequence. By way of non-limiting example, “molecular torches” are a type of self-hybridizing probe that includes distinct regions of self-complementarity (referred to as “the target binding domain” and “the target closing domain”) which are connected by a joining region (e.g., non-nucleotide linker) and which hybridize to each other under predetermined hybridization assay conditions. In a preferred embodiment, molecular torches contain single-stranded base regions in the target binding domain that are from 1 to about 20 bases in length and are accessible for hybridization to a target sequence present in an amplification reaction under strand displacement conditions. Under strand displacement conditions, hybridization of the two complementary regions, which may be fully or partially complementary, of the molecular torch is favored, except in the presence of the target sequence, which will bind to the single-stranded region present in the target binding domain and displace all or a portion of the target closing domain. The target binding domain and the target closing domain of a molecular torch include a detectable label or a pair of interacting labels (e.g., luminescent/quencher) positioned so that a different signal is produced when the molecular torch is self-hybridized than when the molecular torch is hybridized to the target sequence, thereby permitting detection of probe:target duplexes in a test sample in the presence of unhybridized molecular torches. Molecular torches and a variety of types of interacting label pairs are disclosed in U.S. Pat. No. 6,534,274, herein incorporated by reference in its entirety.

Another example of a detection probe having self-complementarity is a “molecular beacon.” Molecular beacons include nucleic acid molecules having a target complementary sequence, an affinity pair (or nucleic acid arms) holding the probe in a closed conformation in the absence of a target sequence present in an amplification reaction, and a label pair that interacts when the probe is in a closed conformation. Hybridization of the target sequence and the target complementary sequence separates the members of the affinity pair, thereby shifting the probe to an open conformation. The shift to the open conformation is detectable due to reduced interaction of the label pair, which may be, for example, a fluorophore and a quencher (e.g., DABCYL and EDANS). Molecular beacons are disclosed in U.S. Pat. Nos. 5,925,517 and 6,150,097, herein incorporated by reference in its entirety.

Other self-hybridizing probes are well known to those of ordinary skill in the art. By way of non-limiting example, probe binding pairs having interacting labels, such as those disclosed in U.S. Pat. No. 5,928,862 (herein incorporated by reference in its entirety) might be adapted for use in the present invention. Probe systems used to detect single nucleotide polymorphisms (SNPs) might also be utilized in the present invention. Additional detection systems include “molecular switches,” as disclosed in U.S. Publ. No. 20050042638, herein incorporated by reference in its entirety. Other probes, such as those comprising intercalating dyes and/or fluorochromes, are also useful for detection of amplification products in the present invention. See, e.g., U.S. Pat. No. 5,814,447 (herein incorporated by reference in its entirety).

C. Protein Detection

The gene fusions of the present invention may be detected as truncated ETS family member proteins or chimeric proteins using a variety of protein techniques known to those of ordinary skill in the art, including but not limited to: protein sequencing; and, immunoassays.

1. Sequencing

Illustrative non-limiting examples of protein sequencing techniques include, but are not limited to, mass spectrometry and Edman degradation.

Mass spectrometry can, in principle, sequence any size protein but becomes computationally more difficult as size increases. A protein is digested by an endoprotease, and the resulting solution is passed through a high pressure liquid chromatography column. At the end of this column, the solution is sprayed out of a narrow nozzle charged to a high positive potential into the mass spectrometer. The charge on the droplets causes them to fragment until only single ions remain. The peptides are then fragmented and the mass-charge ratios of the fragments measured. The mass spectrum is analyzed by computer and often compared against a database of previously sequenced proteins in order to determine the sequences of the fragments. The process is then repeated with a different digestion enzyme, and the overlaps in sequences are used to construct a sequence for the protein.

In the Edman degradation reaction, the peptide to be sequenced is adsorbed onto a solid surface (e.g., a glass fiber coated with polybrene). The Edman reagent, phenylisothiocyanate (PTC), is added to the adsorbed peptide, together with a mildly basic buffer solution of 12% trimethylamine, and reacts with the amine group of the N-terminal amino acid. The terminal amino acid derivative can then be selectively detached by the addition of anhydrous acid. The derivative isomerizes to give a substituted phenylthiohydantoin, which can be washed off and identified by chromatography, and the cycle can be repeated. The efficiency of each step is about 98%, which allows about 50 amino acids to be reliably determined.

2. Immunoassays

Illustrative non-limiting examples of immunoassays include, but are not limited to: immunoprecipitation; Western blot; ELISA; immunohistochemistry; immunocytochemistry; flow cytometry; and, immuno-PCR. Polyclonal or monoclonal antibodies detectably labeled using various techniques known to those of ordinary skill in the art (e.g., colorimetric, fluorescent, chemiluminescent or radioactive) are suitable for use in the immunoassays.

Immunoprecipitation is the technique of precipitating an antigen out of solution using an antibody specific to that antigen. The process can be used to identify protein complexes present in cell extracts by targeting a protein believed to be in the complex. The complexes are brought out of solution by insoluble antibody-binding proteins isolated initially from bacteria, such as Protein A and Protein G. The antibodies can also be coupled to sepharose beads that can easily be isolated out of solution. After washing, the precipitate can be analyzed using mass spectrometry, Western blotting, or any number of other methods for identifying constituents in the complex.

A Western blot, or immunoblot, is a method to detect protein in a given sample of tissue homogenate or extract. It uses gel electrophoresis to separate denatured proteins by mass. The proteins are then transferred out of the gel and onto a membrane, typically polyvinyldiflroride or nitrocellulose, where they are probed using antibodies specific to the protein of interest. As a result, researchers can examine the amount of protein in a given sample and compare levels between several groups.

An ELISA, short for Enzyme-Linked ImmunoSorbent Assay, is a biochemical technique to detect the presence of an antibody or an antigen in a sample. It utilizes a minimum of two antibodies, one of which is specific to the antigen and the other of which is coupled to an enzyme. The second antibody will cause a chromogenic or fluorogenic substrate to produce a signal. Variations of ELISA include sandwich ELISA, competitive ELISA, and ELISPOT. Because the ELISA can be performed to evaluate either the presence of antigen or the presence of antibody in a sample, it is a useful tool both for determining serum antibody concentrations and also for detecting the presence of antigen.

Immunohistochemistry and immunocytochemistry refer to the process of localizing proteins in a tissue section or cell, respectively, via the principle of antigens in tissue or cells binding to their respective antibodies. Visualization is enabled by tagging the antibody with color producing or fluorescent tags. Typical examples of color tags include, but are not limited to, horseradish peroxidase and alkaline phosphatase. Typical examples of fluorophore tags include, but are not limited to, fluorescein isothiocyanate (FITC) or phycoerythrin (PE).

Flow cytometry is a technique for counting, examining and sorting microscopic particles suspended in a stream of fluid. It allows simultaneous multiparametric analysis of the physical and/or chemical characteristics of single cells flowing through an optical/electronic detection apparatus. A beam of light (e.g., a laser) of a single frequency or color is directed onto a hydrodynamically focused stream of fluid. A number of detectors are aimed at the point where the stream passes through the light beam; one in line with the light beam (Forward Scatter or FSC) and several perpendicular to it (Side Scatter (SSC) and one or more fluorescent detectors). Each suspended particle passing through the beam scatters the light in some way, and fluorescent chemicals in the particle may be excited into emitting light at a lower frequency than the light source. The combination of scattered and fluorescent light is picked up by the detectors, and by analyzing fluctuations in brightness at each detector, one for each fluorescent emission peak, it is possible to deduce various facts about the physical and chemical structure of each individual particle. FSC correlates with the cell volume and SSC correlates with the density or inner complexity of the particle (e.g., shape of the nucleus, the amount and type of cytoplasmic granules or the membrane roughness).

Immuno-polymerase chain reaction (IPCR) utilizes nucleic acid amplification techniques to increase signal generation in antibody-based immunoassays. Because no protein equivalence of PCR exists, that is, proteins cannot be replicated in the same manner that nucleic acid is replicated during PCR, the only way to increase detection sensitivity is by signal amplification. The target proteins are bound to antibodies which are directly or indirectly conjugated to oligonucleotides. Unbound antibodies are washed away and the remaining bound antibodies have their oligonucleotides amplified. Protein detection occurs via detection of amplified oligonucleotides using standard nucleic acid detection methods, including real-time methods.

D. Data Analysis

In some embodiments, a computer-based analysis program is used to translate the raw data generated by the detection assay (e.g., the presence, absence, or amount of a given gene fusion or other markers) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., likelihood of cancer being present) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease.

E. In vivo Imaging

The gene fusions of the present invention may also be detected using in vivo imaging techniques, including but not limited to: radionuclide imaging; positron emission tomography (PET); computerized axial tomography, X-ray or magnetic resonance imaging method, fluorescence detection, and chemiluminescent detection. In some embodiments, in vivo imaging techniques are used to visualize the presence of or expression of cancer markers in an animal (e.g., a human or non-human mammal). For example, in some embodiments, cancer marker mRNA or protein is labeled using a labeled antibody specific for the cancer marker. A specifically bound and labeled antibody can be detected in an individual using an in vivo imaging method, including, but not limited to, radionuclide imaging, positron emission tomography, computerized axial tomography, X-ray or magnetic resonance imaging method, fluorescence detection, and chemiluminescent detection. Methods for generating antibodies to the cancer markers of the present invention are described below.

The in vivo imaging methods of the present invention are useful in the diagnosis of cancers that express the cancer markers of the present invention (e.g., prostate cancer). In vivo imaging is used to visualize the presence of a marker indicative of the cancer. Such techniques allow for diagnosis without the use of an unpleasant biopsy. The in vivo imaging methods of the present invention are also useful for providing prognoses to cancer patients. For example, the presence of a marker indicative of cancers likely to metastasize can be detected. The in vivo imaging methods of the present invention can further be used to detect metastatic cancers in other parts of the body.

In some embodiments, reagents (e.g., antibodies) specific for the cancer markers of the present invention are fluorescently labeled. The labeled antibodies are introduced into a subject (e.g., orally or parenterally). Fluorescently labeled antibodies are detected using any suitable method (e.g., using the apparatus described in U.S. Pat. No. 6,198,107, herein incorporated by reference).

In other embodiments, antibodies are radioactively labeled. The use of antibodies for in vivo diagnosis is well known in the art. Sumerdon et al., (Nucl. Med. Biol 17:247-254 [1990] have described an optimized antibody-chelator for the radioimmunoscintographic imaging of tumors using Indium-111 as the label. Griffin et al., (J Clin One 9:631-640 [1991]) have described the use of this agent in detecting tumors in patients suspected of having recurrent colorectal cancer. The use of similar agents with paramagnetic ions as labels for magnetic resonance imaging is known in the art (Lauffer, Magnetic Resonance in Medicine 22:339-342 [1991]). The label used will depend on the imaging modality chosen. Radioactive labels such as Indium-111, Technetium-99m, or Iodine-131 can be used for planar scans or single photon emission computed tomography (SPECT). Positron emitting labels such as Fluorine-19 can also be used for positron emission tomography (PET). For MRI, paramagnetic ions such as Gadolinium (III) or Manganese (II) can be used.

Radioactive metals with half-lives ranging from 1 hour to 3.5 days are available for conjugation to antibodies, such as scandium-47 (3.5 days) gallium-67 (2.8 days), gallium-68 (68 minutes), technetiium-99m (6 hours), and indium-111 (3.2 days), of which gallium-67, technetium-99m, and indium-111 are preferable for gamma camera imaging, gallium-68 is preferable for positron emission tomography.

A useful method of labeling antibodies with such radiometals is by means of a bifunctional chelating agent, such as diethylenetriaminepentaacetic acid (DTPA), as described, for example, by Khaw et al. (Science 209:295 [1980]) for In-111 and Tc-99m, and by Scheinberg et al. (Science 215:1511 [1982]). Other chelating agents may also be used, but the 1-(p-carboxymethoxybenzyl)EDTA and the carboxycarbonic anhydride of DTPA are advantageous because their use permits conjugation without affecting the antibody's immunoreactivity substantially.

Another method for coupling DPTA to proteins is by use of the cyclic anhydride of DTPA, as described by Hnatowich et al. (Int. J. Appl. Radiat. Isot. 33:327 [1982]) for labeling of albumin with In-111, but which can be adapted for labeling of antibodies. A suitable method of labeling antibodies with Tc-99m which does not use chelation with DPTA is the pretinning method of Crockford et al., (U.S. Pat. No. 4,323,546, herein incorporated by reference).

A preferred method of labeling immunoglobulins with Tc-99m is that described by Wong et al. (Int. J. Appl. Radiat. Isot., 29:251 [1978]) for plasma protein, and recently applied successfully by Wong et al. (J. Nucl. Med., 23:229 [1981]) for labeling antibodies.

In the case of the radiometals conjugated to the specific antibody, it is likewise desirable to introduce as high a proportion of the radiolabel as possible into the antibody molecule without destroying its immunospecificity. A further improvement may be achieved by effecting radiolabeling in the presence of the specific cancer marker of the present invention, to insure that the antigen binding site on the antibody will be protected. The antigen is separated after labeling.

In still further embodiments, in vivo biophotonic imaging (Xenogen, Almeda, Calif.) is utilized for in vivo imaging. This real-time in vivo imaging utilizes luciferase. The luciferase gene is incorporated into cells, microorganisms, and animals (e.g., as a fusion protein with a cancer marker of the present invention). When active, it leads to a reaction that emits light. A CCD camera and software is used to capture the image and analyze it.

F. Compositions & Kits

Compositions for use in the diagnostic methods of the present invention include, but are not limited to, probes, amplification oligonucleotides, and antibodies. Particularly preferred compositions detect a product only when a gene fusion is present. These compositions include: a single labeled probe comprising a sequence that hybridizes to the junction at which a 5′ portion from a 5′ fusion partner fuses to a 3′ portion from a 3′ fusion partner (i.e., spans the gene fusion junction); a pair of amplification oligonucleotides wherein the first amplification oligonucleotide comprises a sequence that hybridizes to a 5′ fusion partner and second amplification oligonucleotide comprises a sequence that hybridizes to a 3′ fusion partner; an antibody to an amino-terminally truncated 3′ fusion partner; or, an antibody to a chimeric protein having an amino-terminal portion from a 5′ fusion partner and a carboxy-terminal portion from a 3′ fusion partner. Other useful compositions, however, include: a pair of labeled probes wherein the first labeled probe comprises a sequence that hybridizes to a 5′ fusion partner and the second labeled probe comprises a sequence that hybridizes to a 3′ fusion partner.

Any of these compositions, alone or in combination with other compositions of the present invention, may be provided in the form of a kit. For example, the single labeled probe and pair of amplification oligonucleotides may be provided in a kit for the amplification and detection of gene fusions of the present invention. Kits may further comprise appropriate controls and/or detection reagents. The probe and antibody compositions of the present invention may also be provided in the form of an array.

### IV. Drug Screening Applications

In some embodiments, the present invention provides drug screening assays (e.g., to screen for anticancer drugs). The screening methods of the present invention utilize cancer markers identified using the methods of the present invention (e.g., including but not limited to, gene fusions of the present invention). For example, in some embodiments, the present invention provides methods of screening for compounds that alter (e.g., decrease) the expression of gene fusions. The compounds or agents may interfere with transcription, by interacting, for example, with the promoter region. The compounds or agents may interfere with mRNA produced from the fusion (e.g., by RNA interference, antisense technologies, etc.). The compounds or agents may interfere with pathways that are upstream or downstream of the biological activity of the fusion. In some embodiments, candidate compounds are antisense or interfering RNA agents (e.g., oligonucleotides) directed against cancer markers. In other embodiments, candidate compounds are antibodies or small molecules that specifically bind to a cancer marker regulator or expression products of the present invention and inhibit its biological function.

In one screening method, candidate compounds are evaluated for their ability to alter cancer marker expression by contacting a compound with a cell expressing a cancer marker and then assaying for the effect of the candidate compounds on expression. In some embodiments, the effect of candidate compounds on expression of a cancer marker gene is assayed for by detecting the level of cancer marker mRNA expressed by the cell. mRNA expression can be detected by any suitable method.

In other embodiments, the effect of candidate compounds on expression of cancer marker genes is assayed by measuring the level of polypeptide encoded by the cancer markers. The level of polypeptide expressed can be measured using any suitable method, including but not limited to, those disclosed herein.

Specifically, the present invention provides screening methods for identifying modulators, i.e., candidate or test compounds or agents (e.g., proteins, peptides, peptidomimetics, peptoids, small molecules or other drugs) which bind to cancer markers of the present invention, have an inhibitory (or stimulatory) effect on, for example, cancer marker expression or cancer marker activity, or have a stimulatory or inhibitory effect on, for example, the expression or activity of a cancer marker substrate. Compounds thus identified can be used to modulate the activity of target gene products (e.g., cancer marker genes) either directly or indirectly in a therapeutic protocol, to elaborate the biological function of the target gene product, or to identify compounds that disrupt normal target gene interactions. Compounds that inhibit the activity or expression of cancer markers are useful in the treatment of proliferative disorders, e.g., cancer, particularly prostate cancer.

In one embodiment, the invention provides assays for screening candidate or test compounds that are substrates of a cancer marker protein or polypeptide or a biologically active portion thereof. In another embodiment, the invention provides assays for screening candidate or test compounds that bind to or modulate the activity of a cancer marker protein or polypeptide or a biologically active portion thereof.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin Science 249:404-406 [1990]; Cwirla et al., Proc. Natl. Acad. Sci. 87:6378-6382 [1990]; Felici, J. Mol. Biol. 222:301 [1991]).

In one embodiment, an assay is a cell-based assay in which a cell that expresses a cancer marker mRNA or protein or biologically active portion thereof is contacted with a test compound, and the ability of the test compound to the modulate cancer marker's activity is determined. Determining the ability of the test compound to modulate cancer marker activity can be accomplished by monitoring, for example, changes in enzymatic activity, destruction or mRNA, or the like.

The ability of the test compound to modulate cancer marker binding to a compound, e.g., a cancer marker substrate or modulator, can also be evaluated. This can be accomplished, for example, by coupling the compound, e.g., the substrate, with a radioisotope or enzymatic label such that binding of the compound, e.g., the substrate, to a cancer marker can be determined by detecting the labeled compound, e.g., substrate, in a complex.

Alternatively, the cancer marker is coupled with a radioisotope or enzymatic label to monitor the ability of a test compound to modulate cancer marker binding to a cancer marker substrate in a complex. For example, compounds (e.g., substrates) can be labeled with ¹²⁵I, ³⁵S¹⁴C or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a cancer marker substrate) to interact with a cancer marker with or without the labeling of any of the interactants can be evaluated. For example, a microphysiorneter can be used to detect the interaction of a compound with a cancer marker without the labeling of either the compound or the cancer marker (McConnell et al. Science 257:1906-1912 [1992]). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and cancer markers.

In yet another embodiment, a cell-free assay is provided in which a cancer marker protein or biologically active portion thereof is contacted with a test compound and the ability of the test compound to bind to the cancer marker protein, mRNA, or biologically active portion thereof is evaluated. Preferred biologically active portions of the cancer marker proteins or mRNA to be used in assays of the present invention include fragments that participate in interactions with substrates or other proteins, e.g., fragments with high surface probability scores.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FRET) (see, for example, Lakowicz et al., U.S. Pat. No. 5,631,169; Stavrianopoulos et al., U.S. Pat. No. 4,968,103; each of which is herein incorporated by reference). A fluorophore label is selected such that a first donor molecule's emitted fluorescent energy will be absorbed by a fluorescent label on a second, ‘acceptor’ molecule, which in turn is able to fluoresce due to the absorbed energy.

Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label should be maximal. A FRET binding event can be conveniently measured through standard fluorometric detection means well known in the art (e.g., using a fluorimeter).

In another embodiment, determining the ability of the cancer marker protein or mRNA to bind to a target molecule can be accomplished using real-time Biomolecular Interaction Analysis (BIA) (see, e.g., Sjolander and Urbaniczky, Anal. Chem. 63:2338-2345 [1991] and Szabo et al. Curr. Opin. Struct. Biol. 5:699-705 [1995]). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize cancer markers, an anti-cancer marker antibody or its target molecule to facilitate separation of complexed from non-complexed forms of one or both of the proteins, as well as to accommodate automation of the assay. Binding of a test compound to a cancer marker protein, or interaction of a cancer marker protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase-cancer marker fusion proteins or glutathione-S-transferase/target fusion proteins can be adsorbed onto glutathione Sepharose beads (Sigma Chemical, St. Louis, Mo.) or glutathione-derivatized microtiter plates, which are then combined with the test compound or the test compound and either the non-adsorbed target protein or cancer marker protein, and the mixture incubated under conditions conducive for complex formation (e.g., at physiological conditions for salt and pH). Following incubation, the beads or microtiter plate wells are washed to remove any unbound components, the matrix immobilized in the case of beads, complex determined either directly or indirectly, for example, as described above.

Alternatively, the complexes can be dissociated from the matrix, and the level of cancer markers binding or activity determined using standard techniques. Other techniques for immobilizing either cancer markers protein or a target molecule on matrices include using conjugation of biotin and streptavidin. Biotinylated cancer marker protein or target molecules can be prepared from biotin-NHS(N-hydroxy-succinimide) using techniques known in the art (e.g., biotinylation kit, Pierce Chemicals, Rockford, EL), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-IgG antibody).

This assay is performed utilizing antibodies reactive with cancer marker protein or target molecules but which do not interfere with binding of the cancer markers protein to its target molecule. Such antibodies can be derivatized to the wells of the plate, and unbound target or cancer markers protein trapped in the wells by antibody conjugation. Methods for detecting such complexes, in addition to those described above for the GST-immobilized complexes, include immunodetection of complexes using antibodies reactive with the cancer marker protein or target molecule, as well as enzyme-linked assays which rely on detecting an enzymatic activity associated with the cancer marker protein or target molecule.

Alternatively, cell free assays can be conducted in a liquid phase. In such an assay, the reaction products are separated from unreacted components, by any of a number of standard techniques, including, but not limited to: differential centrifugation (see, for example, Rivas and Minton, Trends Biochem Sci 18:284-7 [1993]); chromatography (gel filtration chromatography, ion-exchange chromatography); electrophoresis (see, e.g., Ausubel et al., eds. Current Protocols in Molecular Biology 1999, J. Wiley: New York); and immunoprecipitation (see, for example, Ausubel et al., eds. Current Protocols in Molecular Biology 1999, J. Wiley: New York). Such resins and chromatographic techniques are known to one skilled in the art (See e.g., Heegaard J. Mol. Recognit 11:141-8 [1998]; Hageand Tweed J. Chromatogr. Biomed. Sci. Appl 699:499-525 [1997]). Further, fluorescence energy transfer may also be conveniently utilized, as described herein, to detect binding without further purification of the complex from solution.

The assay can include contacting the cancer markers protein, mRNA, or biologically active portion thereof with a known compound that binds the cancer marker to form an assay mixture, contacting the assay mixture with a test compound, and determining the ability of the test compound to interact with a cancer marker protein or mRNA, wherein determining the ability of the test compound to interact with a cancer marker protein or mRNA includes determining the ability of the test compound to preferentially bind to cancer markers or biologically active portion thereof, or to modulate the activity of a target molecule, as compared to the known compound.

To the extent that cancer markers can, in vivo, interact with one or more cellular or extracellular macromolecules, such as proteins, inhibitors of such an interaction are useful. A homogeneous assay can be used can be used to identify inhibitors.

For example, a preformed complex of the target gene product and the interactive cellular or extracellular binding partner product is prepared such that either the target gene products or their binding partners are labeled, but the signal generated by the label is quenched due to complex formation (see, e.g., U.S. Pat. No. 4,109,496, herein incorporated by reference, that utilizes this approach for immunoassays). The addition of a test substance that competes with and displaces one of the species from the preformed complex will result in the generation of a signal above background. In this way, test substances that disrupt target gene product-binding partner interaction can be identified. Alternatively, cancer markers protein can be used as a “bait protein” in a two-hybrid assay or three-hybrid assay (see, e.g., U.S. Pat. No. 5,283,317; Zervos et al., Cell 72:223-232 [1993]; Madura et al., J. Biol. Chem. 268.12046-12054 [1993]; Bartel et al., Biotechniques 14:920-924 [1993]; Iwabuchi et al., Oncogene 8:1693-1696 [1993]; and Brent WO 94/10300; each of which is herein incorporated by reference), to identify other proteins, that bind to or interact with cancer markers (“cancer marker-binding proteins” or “cancer marker-bp”) and are involved in cancer marker activity. Such cancer marker-bps can be activators or inhibitors of signals by the cancer marker proteins or targets as, for example, downstream elements of a cancer markers-mediated signaling pathway.

Modulators of cancer markers expression can also be identified. For example, a cell or cell free mixture is contacted with a candidate compound and the expression of cancer marker mRNA or protein evaluated relative to the level of expression of cancer marker mRNA or protein in the absence of the candidate compound. When expression of cancer marker mRNA or protein is greater in the presence of the candidate compound than in its absence, the candidate compound is identified as a stimulator of cancer marker mRNA or protein expression. Alternatively, when expression of cancer marker mRNA or protein is less (i.e., statistically significantly less) in the presence of the candidate compound than in its absence, the candidate compound is identified as an inhibitor of cancer marker mRNA or protein expression. The level of cancer markers mRNA or protein expression can be determined by methods described herein for detecting cancer markers mRNA or protein.

A modulating agent can be identified using a cell-based or a cell free assay, and the ability of the agent to modulate the activity of a cancer markers protein can be confirmed in vivo, e.g., in an animal such as an animal model for a disease (e.g., an animal with prostate cancer or metastatic prostate cancer; or an animal harboring a xenograft of a prostate cancer from an animal (e.g., human) or cells from a cancer resulting from metastasis of a prostate cancer (e.g., to a lymph node, bone, or liver), or cells from a prostate cancer cell line.

This invention further pertains to novel agents identified by the above-described screening assays (See e.g., below description of cancer therapies). Accordingly, it is within the scope of this invention to further use an agent identified as described herein (e.g., a cancer marker modulating agent, an antisense cancer marker nucleic acid molecule, a siRNA molecule, a cancer marker specific antibody, or a cancer marker-binding partner) in an appropriate animal model (such as those described herein) to determine the efficacy, toxicity, side effects, or mechanism of action, of treatment with such an agent. Furthermore, novel agents identified by the above-described screening assays can be, e.g., used for treatments as described herein.

### V. Therapeutic Applications

In some embodiments, the present invention provides therapies for cancer (e.g., prostate cancer). In some embodiments, therapies directly or indirectly target gene fusions of the present invention.

A. RNA Interference and Antisense Therapies

In some embodiments, the present invention targets the expression of gene fusions. For example, in some embodiments, the present invention employs compositions comprising oligomeric antisense or RNAi compounds, particularly oligonucleotides (e.g., those identified in the drug screening methods described above), for use in modulating the function of nucleic acid molecules encoding cancer markers of the present invention, ultimately modulating the amount of cancer marker expressed.

1. RNA Interference (RNAi)

In some embodiments, RNAi is utilized to inhibit fusion protein function. RNAi represents an evolutionary conserved cellular defense for controlling the expression of foreign genes in most eukaryotes, including humans. RNAi is typically triggered by double-stranded RNA (dsRNA) and causes sequence-specific mRNA degradation of single-stranded target RNAs homologous in response to dsRNA. The mediators of mRNA degradation are small interfering RNA duplexes (siRNAs), which are normally produced from long dsRNA by enzymatic cleavage in the cell. siRNAs are generally approximately twenty-one nucleotides in length (e.g. 21-23 nucleotides in length), and have a base-paired structure characterized by two nucleotide 3′-overhangs. Following the introduction of a small RNA, or RNAi, into the cell, it is believed the sequence is delivered to an enzyme complex called RISC(RNA-induced silencing complex). RISC recognizes the target and cleaves it with an endonuclease. It is noted that if larger RNA sequences are delivered to a cell, RNase III enzyme (Dicer) converts longer dsRNA into 21-23 nt ds siRNA fragments. In some embodiments, RNAi oligonucleotides are designed to target the junction region of fusion proteins.

Chemically synthesized siRNAs have become powerful reagents for genome-wide analysis of mammalian gene function in cultured somatic cells. Beyond their value for validation of gene function, siRNAs also hold great potential as gene-specific therapeutic agents (Tuschl and Borkhardt, Molecular Intervent. 2002; 2(3):158-67, herein incorporated by reference).

The transfection of siRNAs into animal cells results in the potent, long-lasting post-transcriptional silencing of specific genes (Caplen et al, Proc Natl Acad Sci U.S.A. 2001; 98: 9742-7; Elbashir et al., Nature. 2001; 411:494-8; Elbashir et al., Genes Dev. 2001; 15: 188-200; and Elbashir et al., EMBO J. 2001; 20: 6877-88, all of which are herein incorporated by reference). Methods and compositions for performing RNAi with siRNAs are described, for example, in U.S. Pat. No. 6,506,559, herein incorporated by reference.

siRNAs are extraordinarily effective at lowering the amounts of targeted RNA, and by extension proteins, frequently to undetectable levels. The silencing effect can last several months, and is extraordinarily specific, because one nucleotide mismatch between the target RNA and the central region of the siRNA is frequently sufficient to prevent silencing (Brummelkamp et al, Science 2002; 296:550-3; and Holen et al, Nucleic Acids Res. 2002; 30:1757-66, both of which are herein incorporated by reference).

An important factor in the design of siRNAs is the presence of accessible sites for siRNA binding. Bahoia et al., (J. Biol. Chem., 2003; 278: 15991-15997; herein incorporated by reference) describe the use of a type of DNA array called a scanning array to find accessible sites in mRNAs for designing effective siRNAs. These arrays comprise oligonucleotides ranging in size from monomers to a certain maximum, usually Corners, synthesized using a physical barrier (mask) by stepwise addition of each base in the sequence. Thus the arrays represent a full oligonucleotide complement of a region of the target gene. Hybridization of the target mRNA to these arrays provides an exhaustive accessibility profile of this region of the target mRNA. Such data are useful in the design of antisense oligonucleotides (ranging from 7 mers to 25 mers), where it is important to achieve a compromise between oligonucleotide length and binding affinity, to retain efficacy and target specificity (Sohail et al, Nucleic Acids Res., 2001; 29(10): 2041-2045). Additional methods and concerns for selecting siRNAs are described for example, in WO 05054270, WO05038054A1, WO03070966A2, J Mol. Biol. 2005 May 13; 348(4):883-93, J Mol. Biol. 2005 May 13; 348(4):871-81, and Nucleic Acids Res. 2003 Aug. 1; 31(15):4417-24, each of which is herein incorporated by reference in its entirety. In addition, software (e.g., the MWG online siMAX siRNA design tool) is commercially or publicly available for use in the selection of siRNAs.

2. Antisense

In other embodiments, fusion protein expression is modulated using antisense compounds that specifically hybridize with one or more nucleic acids encoding cancer markers of the present invention. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of the expression of cancer markers of the present invention. In the context of the present invention, “modulation” means either an increase (stimulation) or a decrease (inhibition) in the expression of a gene. For example, expression may be inhibited to potentially prevent tumor proliferation.

The present invention also includes pharmaceutical compositions and formulations that include the antisense compounds of the present invention as described below.

B. Gene Therapy

The present invention contemplates the use of any genetic manipulation for use in modulating the expression of gene fusions of the present invention. Examples of genetic manipulation include, but are not limited to, gene knockout (e.g., removing the fusion gene from the chromosome using, for example, recombination), expression of antisense constructs with or without inducible promoters, and the like. Delivery of nucleic acid construct to cells in vitro or in vivo may be conducted using any suitable method. A suitable method is one that introduces the nucleic acid construct into the cell such that the desired event occurs (e.g., expression of an antisense construct). Genetic therapy may also be used to deliver siRNA or other interfering molecules that are expressed in vivo (e.g., upon stimulation by an inducible promoter (e.g., an androgen-responsive promoter)).

Introduction of molecules carrying genetic information into cells is achieved by any of various methods including, but not limited to, directed injection of naked DNA constructs, bombardment with gold particles loaded with said constructs, and macromolecule mediated gene transfer using, for example, liposomes, biopolymers, and the like. Preferred methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer into a variety of solid tumors in animal models and into human solid tumor xenografts in immune-deficient mice. Examples of adenoviral vectors and methods for gene transfer are described in PCT publications WO 00/12738 and WO 00/09675 and U.S. Pat. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety.

Vectors may be administered to subject in a variety of ways. For example, in some embodiments of the present invention, vectors are administered into tumors or tissue associated with tumors using direct injection. In other embodiments, administration is via the blood or lymphatic circulation (See e.g., PCT publication 99/02685 herein incorporated by reference in its entirety). Exemplary dose levels of adenoviral vector are preferably 10⁸ to 10¹¹ vector particles added to the perfusate.

C. Antibody Therapy

In some embodiments, the present invention provides antibodies that target prostate tumors that express a gene fusion of the present invention. Any suitable antibody (e.g., monoclonal, polyclonal, or synthetic) may be utilized in the therapeutic methods disclosed herein. In preferred embodiments, the antibodies used for cancer therapy are humanized antibodies. Methods for humanizing antibodies are well known in the art (See e.g., U.S. Pat. Nos. 6,180,370, 5,585,089, 6,054,297, and 5,565,332; each of which is herein incorporated by reference).

In some embodiments, the therapeutic antibodies comprise an antibody generated against a gene fusion of the present invention, wherein the antibody is conjugated to a cytotoxic agent. In such embodiments, a tumor specific therapeutic agent is generated that does not target normal cells, thus reducing many of the detrimental side effects of traditional chemotherapy. For certain applications, it is envisioned that the therapeutic agents will be pharmacologic agents that will serve as useful agents for attachment to antibodies, particularly cytotoxic or otherwise anticellular agents having the ability to kill or suppress the growth or cell division of endothelial cells. The present invention contemplates the use of any pharmacologic agent that can be conjugated to an antibody, and delivered in active form. Exemplary anticellular agents include chemotherapeutic agents, radioisotopes, and cytotoxins. The therapeutic antibodies of the present invention may include a variety of cytotoxic moieties, including but not limited to, radioactive isotopes (e.g., iodine-131, iodine-123, technicium-99m, indium-111, rhenium-188, rhenium-186, gallium-67, copper-67, yttrium-90, iodine-125 or astatine-211), hormones such as a steroid, antimetabolites such as cytosines (e.g., arabinoside, fluorouracil, methotrexate or aminopterin; an anthracycline; mitomycin C), vinca alkaloids (e.g., demecolcine; etoposide; mithramycin), and antitumor alkylating agent such as chlorambucil or melphalan. Other embodiments may include agents such as a coagulant, a cytokine, growth factor, bacterial endotoxin or the lipid A moiety of bacterial endotoxin. For example, in some embodiments, therapeutic agents will include plant-, fungus- or bacteria-derived toxin, such as an A chain toxins, a ribosome inactivating protein, α-sarcin, aspergillin, restrictocin, a ribonuclease, diphtheria toxin or pseudomonas exotoxin, to mention just a few examples. In some preferred embodiments, deglycosylated ricin A chain is utilized.

In any event, it is proposed that agents such as these may, if desired, be successfully conjugated to an antibody, in a manner that will allow their targeting, internalization, release or presentation to blood components at the site of the targeted tumor cells as required using known conjugation technology (See, e.g., Ghose et al., Methods Enzymol., 93:280 [1983]).

For example, in some embodiments the present invention provides immunotoxins targeted a cancer marker of the present invention (e.g., ERG or ETV1 fusions). Immunotoxins are conjugates of a specific targeting agent typically a tumor-directed antibody or fragment, with a cytotoxic agent, such as a toxin moiety. The targeting agent directs the toxin to, and thereby selectively kills, cells carrying the targeted antigen. In some embodiments, therapeutic antibodies employ crosslinkers that provide high in vivo stability (Thorpe et al., Cancer Res., 48:6396 [1988]).

In other embodiments, particularly those involving treatment of solid tumors, antibodies are designed to have a cytotoxic or otherwise anticellular effect against the tumor vasculature, by suppressing the growth or cell division of the vascular endothelial cells. This attack is intended to lead to a tumor-localized vascular collapse, depriving the tumor cells, particularly those tumor cells distal of the vasculature, of oxygen and nutrients, ultimately leading to cell death and tumor necrosis.

In preferred embodiments, antibody based therapeutics are formulated as pharmaceutical compositions as described below. In preferred embodiments, administration of an antibody composition of the present invention results in a measurable decrease in cancer (e.g., decrease or elimination of tumor).

D. Pharmaceutical Compositions

The present invention further provides pharmaceutical compositions (e.g., comprising pharmaceutical agents that modulate the expression or activity of gene fusions of the present invention). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Certain embodiments of the invention provide pharmaceutical compositions containing (a) one or more antisense compounds and (b) one or more other chemotherapeutic agents that function by a non-antisense mechanism. Examples of such chemotherapeutic agents include, but are not limited to, anticancer drugs such as daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxuridine (5-FUdR), methotrexate (MTX), colchicine, vincristine, vinblastine, etoposide, teniposide, cisplatin and diethylstilbestrol (DES). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Other non-antisense chemotherapeutic agents are also within the scope of this invention. Two or more combined compounds may be used together or sequentially.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligonucleotide is administered in maintenance doses, ranging from 0.01 μg to 100 g per kg of body weight, once or more daily, to once every 20 years.

### VI. Transgenic Animals

The present invention contemplates the generation of transgenic animals comprising an exogenous cancer marker gene (e.g., gene fusion) of the present invention or mutants and variants thereof (e.g., truncations or single nucleotide polymorphisms). In preferred embodiments, the transgenic animal displays an altered phenotype (e.g., increased or decreased presence of markers) as compared to wild-type animals. Methods for analyzing the presence or absence of such phenotypes include but are not limited to, those disclosed herein. In some preferred embodiments, the transgenic animals further display an increased or decreased growth of tumors or evidence of cancer.

The transgenic animals of the present invention find use in drug (e.g., cancer therapy) screens. In some embodiments, test compounds (e.g., a drug that is suspected of being useful to treat cancer) and control compounds (e.g., a placebo) are administered to the transgenic animals and the control animals and the effects evaluated.

The transgenic animals can be generated via a variety of methods. In some embodiments, embryonal cells at various developmental stages are used to introduce transgenes for the production of transgenic animals. Different methods are used depending on the stage of development of the embryonal cell. The zygote is the best target for micro-injection. In the mouse, the male pronucleus reaches the size of approximately 20 micrometers in diameter that allows reproducible injection of 1-2 picoliters (pl) of DNA solution. The use of zygotes as a target for gene transfer has a major advantage in that in most cases the injected DNA will be incorporated into the host genome before the first cleavage (Brinster et al., Proc. Natl. Acad. Sci. USA 82:4438-4442 [1985]). As a consequence, all cells of the transgenic non-human animal will carry the incorporated transgene. This will in general also be reflected in the efficient transmission of the transgene to offspring of the founder since 50% of the germ cells will harbor the transgene. U.S. Pat. No. 4,873,191 describes a method for the micro-injection of zygotes; the disclosure of this patent is incorporated herein in its entirety.

In other embodiments, retroviral infection is used to introduce transgenes into a non-human animal. In some embodiments, the retroviral vector is utilized to transfect oocytes by injecting the retroviral vector into the perivitelline space of the oocyte (U.S. Pat. No. 6,080,912, incorporated herein by reference). In other embodiments, the developing non-human embryo can be cultured in vitro to the blastocyst stage. During this time, the blastomeres can be targets for retroviral infection (Janenich, Proc. Natl. Acad. Sci. USA 73:1260 [1976]). Efficient infection of the blastomeres is obtained by enzymatic treatment to remove the zona pellucida (Hogan et al., in Manipulating the Mouse Embryo, Cold Spring Harbor Laboratory Press, Cold Spring Harbor, N.Y. [1986]). The viral vector system used to introduce the transgene is typically a replication-defective retrovirus carrying the transgene (Jahner et al., Proc. Natl. Acad. Sci. USA 82:6927 [1985]). Transfection is easily and efficiently obtained by culturing the blastomeres on a monolayer of virus-producing cells (Stewart, et al., EMBO J., 6:383 [1987]). Alternatively, infection can be performed at a later stage. Virus or virus-producing cells can be injected into the blastocoele (Jahner et al., Nature 298:623 [1982]). Most of the founders will be mosaic for the transgene since incorporation occurs only in a subset of cells that form the transgenic animal. Further, the founder may contain various retroviral insertions of the transgene at different positions in the genome that generally will segregate in the offspring. In addition, it is also possible to introduce transgenes into the germline, albeit with low efficiency, by intrauterine retroviral infection of the midgestation embryo (Jahner et al., supra [1982]). Additional means of using retroviruses or retroviral vectors to create transgenic animals known to the art involve the micro-injection of retroviral particles or mitomycin C-treated cells producing retrovirus into the perivitelline space of fertilized eggs or early embryos (PCT International Application WO 90/08832 [1990], and Haskell and Bowen, Mol. Reprod. Dev., 40:386 [1995]).

In other embodiments, the transgene is introduced into embryonic stem cells and the transfected stem cells are utilized to form an embryo. ES cells are obtained by culturing pre-implantation embryos in vitro under appropriate conditions (Evans et al., Nature 292:154 [1981]; Bradley et al., Nature 309:255 [1984]; Gossler et al., Proc. Acad. Sci. USA 83:9065 [1986]; and Robertson et al., Nature 322:445 [1986]). Transgenes can be efficiently introduced into the ES cells by DNA transfection by a variety of methods known to the art including calcium phosphate co-precipitation, protoplast or spheroplast fusion, lipofection and DEAE-dextran-mediated transfection. Transgenes may also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal (for review, See, Jaenisch, Science 240:1468 [1988]). Prior to the introduction of transfected ES cells into the blastocoel, the transfected ES cells may be subjected to various selection protocols to enrich for ES cells which have integrated the transgene assuming that the transgene provides a means for such selection. Alternatively, the polymerase chain reaction may be used to screen for ES cells that have integrated the transgene. This technique obviates the need for growth of the transfected ES cells under appropriate selective conditions prior to transfer into the blastocoel.

In still other embodiments, homologous recombination is utilized to knock-out gene function or create deletion mutants (e.g., truncation mutants). Methods for homologous recombination are described in U.S. Pat. No. 5,614,396, incorporated herein by reference.

### EXPERIMENTAL

The following examples are provided in order to demonstrate and further illustrate certain preferred embodiments and aspects of the present invention and are not to be construed as limiting the scope thereof.

### Example 1

This example describes materials and methods used for Example 2.

### Samples and Cell Lines

The benign immortalized prostate cell line RWPE and the prostate cancer cell line LNCaP was obtained from the American Type Culture Collection. Primary benign prostatic epithelial cells (PrEC) were obtained from Cambrex Bio Science. VCaP was derived from a vertebral metastasis from a patient with hormonerefractory metastatic prostate cancer (Korenchuk et al., In vivo (Athens, Greece) 15:163 [2001]).

Androgen stimulation experiment was carried out with LNCaP and VCaP cells grown in charcoal-stripped serum containing media for 24 h, before treatment with 1% ethanol or 1 nM of methyltrienolone (R1881, NEN Life Science Products) dissolved in ethanol, for 24 and 48 h. Total RNA was isolated with RNeasy mini kit (Qiagen) according to the manufacturer's instructions.

Prostate tissues were obtained from the radical prostatectomy series at the University of Michigan and from the Rapid Autopsy Program (Rubin et al., Clin. Cancer Res. 6:1038 [2000]), University of Michigan Prostate Cancer Specialized Program of Research Excellence Tissue Core.

### 454 FLX Sequencing

PolyA+ RNA was purified from 50 μg total RNA using two rounds of selection on oligo-dT containing paramagnetic beads using Dynabeads mRNA Purification Kit (Dynal Biotech, Oslo, Norway), according to the manufacturer's instructions. 200 ng mRNA was fragmented at 82° C. in Fragmentation Buffer (40 mM Tris-Acetate, 100 mM Potassium Acetate, 31.5 mM Magnesium Acetate, pH 8.1) for 2 minutes. First strand cDNA library was prepared using Superscript II (Invitrogen) according to standard protocols and directional adaptors were ligated to the cDNA ends for clonal amplification and sequencing on the Genome Sequencer FLX. The 5′-end Adaptor A has a 5′ overhang of 5 nucleotides and the 3′-end Adaptor B has a 3′ overhang of 6 random nucleotides, as shown:

(SEQ ID NO: 1)
5′-NANNACTGATGGCGCGAGGGAGGC-3′
(SEQ ID NO: 2)
GACTACCGCGCTCCCTCCG-5′
(SEQ ID NO: 3)
5′-biotin-GCCTTGCCAGCCCGCTCAGNNNNNN-P-3′
(SEQ ID NO: 4)
3′-CGGAACGGTCGGGCGAGTC

The adaptor ligation reaction was carried out in Quick Ligase Buffer (New England Biolabs, Ipswich, Mass.) containing 1.67 μM of the Adaptor A, 6.67 μM of the Adaptor B and 2000 units of T4 DNA Ligase (New England Biolabs, Ipswich, Mass.) at 37° C. for 2 hours. Adapted library was recovered with 0.05% Sera-Mag30 streptavidin beads (Seradyn Inc, Indianapolis, Ind.) according to manufacturer's instructions. Finally, the sscDNA library was purified twice with RNAClean (Agencourt, Beverly, Mass.) as per the manufacturer's directions except the amount of beads was reduced to 1.6× the volume of the sample. The purified sscDNA library was analyzed on an RNA 6000 Pico chip on a 2100 Bioanalyzer (Agilent Technologies, Santa Clara, Calif.) to confirm a size distribution between 450 to 750 nucleotides, and quantified with Quant-iT Ribogreen RNA Assay Kit (Invitrogen Corporation, Carlsbad, Calif.) on a Synergy HT (Bio-Tek Instruments Inc, Winooski, Vt.) instrument following the manufacturer's instructions. The library was PCR amplified with 2 μM each of Primer A (5′-GCC TCC CTC GCG CCA-3; SEQ ID NO:5) and Primer B (5′-GCC TTG CCA GCC CGC-3; SEQ ID NO:6), 400 μM dNTPs, 1× Advantage 2 buffer and 1 μl of Advantage 2 polymerase mix (Clontech, Mountain View, Calif.). The amplification reaction was performed at: 96° C. for 4 min; 94° C. for 30 sec, 64° C. for 30 sec, repeating steps 2 and 3 for a total of 20 cycles, followed by 68° C. for 3 minutes. The samples were purified using AMPure beads and diluted to a final working concentration of 200,000 molecules per pl. Emulsion beads for sequencing were generated using Sequencing emPCR Kit II and Kit III and sequencing was carried out using 600,000 beads.

### Normalization by Subtraction

mRNA from the prostate cancer cell line VCaP was hybridized with the subtractor cell line LNCaP 1st-strand cDNA immobilised on magnetic beads (Dynabeads, Invitrogen), according to the manufacturer's instructions. Transcripts common to both the cells were captured and removed by magnetic separation of bead-bound subtractor cDNA and the subtracted VCaP mRNA left in the supernatant was recovered by precipitation and used for generating sequencing library as described. Efficiency of normalization was assessed by qRT-PCR assay of levels of select transcripts in the sample before and after the subtraction.

### Illumina Genome Analyzer Sequencing

200 ng mRNA was fragmented at 70° C. for 5 min in a Fragmentation buffer (Ambion), and converted to first strand cDNA using Superscript III (Invitrogen), followed by second strand cDNA synthesis using E coli DNA pol I (Invitrogen). The double stranded cDNA library was further processed by Illumina Genomic DNA Sample Prep kit; processing involved end repair using T4 DNA polymerase, Klenow DNA polymerase, and T4 Polynucleotide kinase followed by a single <A> base addition using Klenow 3′ to 5′ exo-polymerase, and was ligated with Illumina's adaptor oligo mix using T4 DNA ligase. Adaptor ligated library was size selected by separating on a 4% agarose gel and cutting out the library smear at 200 bp (+/−25 bp). The library was PCR amplified by Phu polymerase (Stratagene), and purified by Qiaquick PCR purification kit (Qiagen). The library was quantified with Quant-iT Picogreen dsDNA Assay Kit (Invitrogen Corporation, Carlsbad, Calif.) on a Modulus™ Single Tube Luminometer (Turner Biosystems, Sunnyvale, Calif.) following the manufacturer's instructions. 10 nM library was used to prepare flowcells with approximately 30,000 clusters per lane.

### Sequence Datasets

Human genome build 18 (hg18) was used as a reference genome. All UCSC and Refseq transcripts were downloaded from the UCSC genome browser (Karolchik et al. Nucleic Acids Res. 32:D493 [2004]). Sequences of previously identified TMPRSS2-ERGa fusion transcript (Genbank accession: DQ204772) and BCR-ABL1 fusion transcript (Genbank accession: M30829) were used for reference.

### Short Read Chimera Discovery

Short reads that do not completely align to the human genome, Refseq genes, mitochondrial, ribosomal, or contaminant sequences are categorized as non-mapping. For many chimeras it was expected that there would be a larger portion mapping to a fusion partner (major alignment), and smaller portion aligning to the second partner (minor alignment). The approach was therefore divided into two phases which focused on first identifying the major alignment and then performing a more exhaustive approach for identifying the minor alignment. In the first phase all non-mapping reads are aligned against all exons of Refseq genes using Vmatch, a pattern matching program (Abouelhoda et al., J. Discrete Algorithsms 1:53[2004]). Only reads that have an alignment of 12 or more nucleotides to an exon boundary are kept as potential chimeras. In the second phase, the non-mapping portion of the remaining reads are then mapped to all possible exon boundaries using a Perl script that utilizes regular expressions to detect alignments of as few as six nucleotides. Only those short reads that show partial alignment to exon boundaries of two separate genes are categorized as chimeras. It is possible to have a chimera that has 28 nucleotides aligning to gene x and 8 nucleotides that align to gene y and z because the 8-mer does not provide enough sequence resolution to distinguish between gene y and gene z. Therefore this would be categorized as two individual chimeras. If a sequence forms more than five chimeras it is discarded because it is ambiguous. To minimize false positives, a predicted gene fusion event was required to have at least two supporting chimeras.

### Long and Short Read Integrated Chimera Discovery

All 454 reads are aligned against the human Refseq collection using BLAT, a rapid mRNA/DNA alignment tool (Kent, Gen. Res. 12:656 [2002])). Using a Perl script, the BLAT output files were parsed to detect potential chimeric reads. A read is categorized as completely aligning if it shows greater than 90% alignment to a known Refseq transcript. These are then discarded as they almost completely align and therefore are not characteristic of a chimera. From the remaining reads, it was desirable to query for reads having partial alignment, with minimal overlap, to two Refseq transcripts representing putative chimeras. To accomplish this, all possible BLAT alignments were iterated for a putative chimera, extracting only those partial alignments that have no more than a six nucleotide, or two codon, overlap. This step reduces false positive chimeras introduced by repetitive regions, large gene families, and conserved domains. Additionally, while the approach tolerates overlap between the partial alignments, it filters those having more than ten or more nucleotides between the partial alignments. The short reads (36 nucleotides) generated from the Illumina platform are parsed by aligning them against the Refseq database and the human genome using Eland, an alignment tool for short reads. Reads that align completely or fail quality control are removed leaving only the “non-mapping” reads; a rich source for chimeras. These non-mapping short reads are subsequently aligned against all putative long read chimeras (obtained as described above) using Vmatch20, a pattern matching program. A Perl script is used to parse the Vmatch output to extract only those reads that span the fusion boundary by at least three nucleotides on each side. Following this integration, the remaining putative chimeras are categorized as inter- or intra-chromosomal chimeras based on whether the partial alignments are located on different or the same chromosomes, respectively. Those intra-chromosomal chimeras that have partial alignments to adjacent genes are believed to be the product of co-transcription of adjacent genes coupled with intergenic splicing (CoTIS) (Communi et al., J. Biol. Chem. 276:16561 [2001]), alternatively known as read-throughs. The remaining intra-chromosomal and all inter-chromosomal chimeras are considered candidate gene fusions.

One additional source of false positive chimeras could be an unknown transcript that is not in Refseq. Due to its absence in the Refseq database, the corresponding long read would not be able to show a complete alignment, but instead show partial hits. Subsequently, short reads spanning this transcript would naturally validate the artificially produced fusion boundary. Therefore, to remove these candidates, all of the chimeras were aligned against the human genome using BLAT. If the long read had greater than 90% alignment to one genomic location, it was considered a novel transcript rather than a chimeric read. The remaining chimeras were given a score which was calculated by multiplying the long read coverage spanning the fusion boundary against the short read coverage spanning the fusion boundary.

### Coverage Analysis

Transcript coverage for every gene locus was calculated from the total number of passing filter reads that mapped, via ELAND, to exons. The total count of these reads was multiplied by the read length and divided by the longest transcript isoform of the gene as determined by the sum of all exon lengths as defined in the UCSC knownGene table (March 2006 assembly). Nucleotide coverage was determined by enumerating the total reads, based on ELAND mappings, at every nucleotide position within a non-redundant set of exons from all possible UCSC transcript isoforms.

### Array CGH Analysis

Oligonucleotide comparative genomic hybridization is a high-resolution method to detect unbalanced copy number changes at whole genome level. Competitive hybridization of differentially labeled tumor and reference DNA to oligonucleotide printed in an array format (Agilent Technologies, USA) and analysis of fluorescent intensity for each probe will detect the copy number changes in the tumor sample relative to normal reference genome. Genomic breakpoints were identified at regions with a change in copy number level of at least one copy (log ratio±0.5) for gains and losses involving more than one probe representing each genomic interval as detected by the aberration detection method (ADM) in CGH analytics algorithm.

### Real Time PCR Validation

Quantitative PCR (QPCR) was performed using Power SYBR Green Mastermix (Applied Biosystems, Foster City, Calif.) on an Applied Biosystems Step One Plus Real Time PCR System as described (Tomlins et al., Nature 448:595 [2007]). All oligonucleotide primers were synthesized by Integrated DNA Technologies (Coralville, Iowa). All assays were performed in duplicate or triplicate and results were plotted as average fold change relative to GAPDH.

Quantitative PCR for SLC45A3-ELK4 was carried out by Taqman assay method using fusion specific primers and Probe #7 of Universal Probe Library (UPL), Human (Roche) as the internal oligonucleotide, according to manufacturer's instructions. PGK1 was used as housekeeping control gene for UPL based Taqman assay (Roche), as per manufacturer's instructions. HMBS (Applied Biosystems, Taqman assay Hs00609297_ml) was used as housekeeping gene control for Taqman assays according to standard protocols (Applied Biosystems).

### Fluorescence In Situ Hybridization (FISH)

FISH hybridizations were performed on VCaP, LNCaP, and FFPE tumor and normal tissues. BAC clones were selected from UCSC genome browser. Following colony purification midi prep DNA was prepared using QiagenTips-100 (Qiagen, USA). DNA was labeled by nick translation labeling with biotin-16-dUTP and digoxigenin-11-dUTP (Roche, USA). Probe DNA was precipitated and dissolved in hybridization mixture containing 50% formamide, 2×SSC, 10% dextran sulphate, and 1% Denhardts solution. About 200 ng of labeled probes was hybridized to normal human chromosomes to confirm the map position of each BAC clone. FISH signals were obtained using anti digoxigenin-fluorescein and alexa fluor594 conjugate for green and red colors respectively. Fluorescence images were captured using a high resolution CCD camera controlled by ISIS image processing software (Metasystems, Germany).

### Affymetrix Genome-Wide Human SNP Array 6.0

1 μg each of genomic DNA samples was sent to Affymetrix service centers (Center for Molecular Medicine, Grand Rapid, Mich. and Vanderbilt Affymetrix Genotyping Core, Nashville, Tenn.) for genomic level analysis of 15 samples on the Genome-Wide Human SNP Array 6.0. Copy number analysis was conducted using the Affymetrix Genotyping Console software and visualizations were generated by the Genotyping Console (GTC) browser.

### Example 2

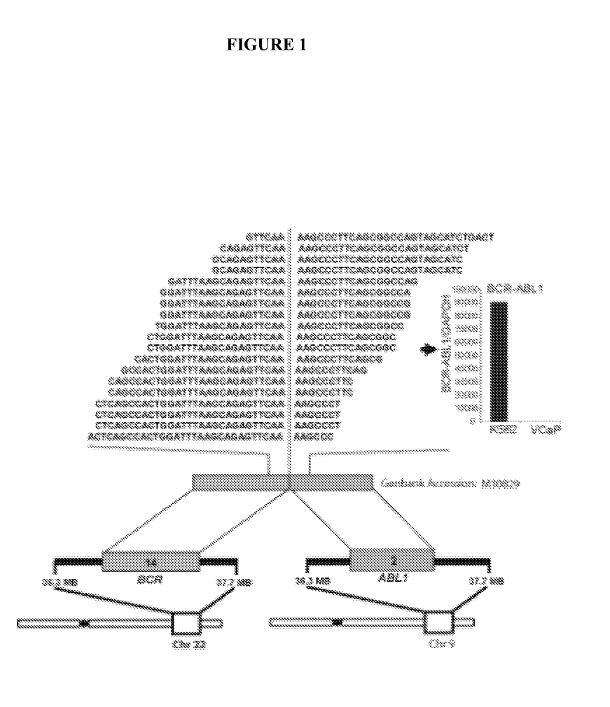

As a proof of concept during experiments conducted during the course of the present invention whole transcriptome sequencing of the chronic myelogenous leukemia cell line, K562, harboring the classical gene fusion, BCR-ABL1 (Shtivelman et al., Nature 315:550 [1985]) was carried out. Using the Illumina Genome Analyzer, 66.9 million reads of 36 nucleotides in length were generated and screened for the presence of reads showing partial alignment to exon boundaries from two different genes. While this approach was able to detect BCR-ABL1, it was one among a set of 111 other chimeras (with at least 2 reads). Thus, in a de novo discovery mode, it would be difficult to pin-point the BCR-ABL1 fusion in the background of the other putative chimeras. However, when the known fusion junction of BCR-ABL1 (Genbank No. M30829) was used as the reference sequence, 19 chimeric reads were detected (FIG. 1). Thus, an integrative approach was used for chimera detection, utilizing short read sequencing technology for obtaining deep sequence data and long read technology (Roche 454 sequencing platform) to provide reference sequences for mapping candidate fusion genes.

A factor in transcriptome sequencing was whether chimeric transcripts could be detected in the background of highly abundant house-keeping genes (i.e., would cDNA normalization be required). To address this, sequences were compared from normalized and non-normalized cDNA libraries of the prostate cancer cell line VCaP, which harbors the gene fusion TMPRSS2-ERG (TABLE 1). Overall, the normalized library showed an approximately 3.6-fold reduction in the total number of chimeras nominated. Furthermore, while it was expected that the normalized library would enrich for the TMPRSS2-ERG gene fusion, it failed to reveal any TMPRSS2-ERG chimeras indicating that normalization would not provide benefit in these analyses.

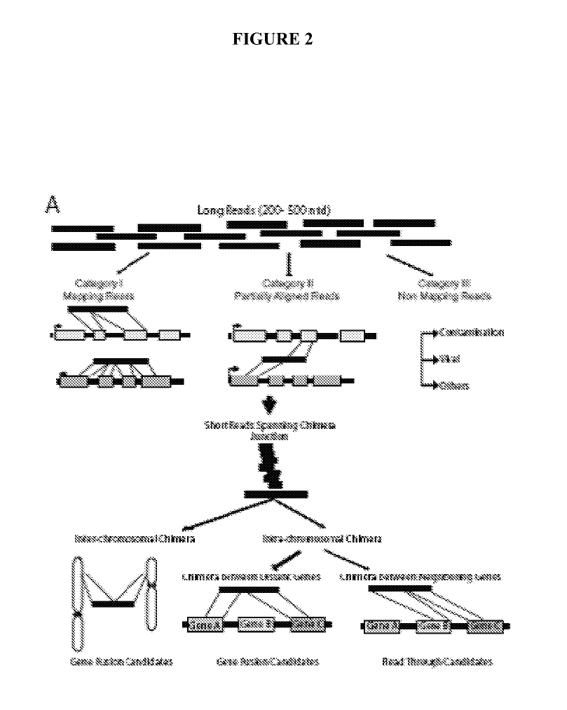

To assess the feasibility of using massively parallel transcriptome sequencing to identify novel gene fusions, non-normalized cDNA libraries were generated from the prostate cancer cell lines VCaP and LNCaP, and a benign immortalized prostate cell line RWPE. As a first step, using the Roche 454 platform, a total of 551,912 VCaP, 244,984 LNCaP, and 826,624 RWPE transcriptome sequence reads were generated, averaging 229.4 nucleotides. These were categorized as completely aligning, partially aligning, or nonmapping to the human reference database (FIG. 2). Sequence reads that showed partial alignments to two genes were nominated as first pass candidate chimeras. This yielded 428 VCaP, 247 LNCaP, and 83 RWPE candidates.

Admittedly, many of these chimeric sequences could be a result of trans-splicing (Takahara et al., Mol. Cell 18:245 [2005]) or co-transcription of adjacent genes coupled with intergenic splicing (Communi et al., J. Biol. Chem. 276:16561 [2001]), or simply, an artifact of the sequencing protocol. Among the 428 VCaP candidates, only one read spanned the TMPRSS2-ERG fusion junction using the long read sequencing platform (TABLE 2).

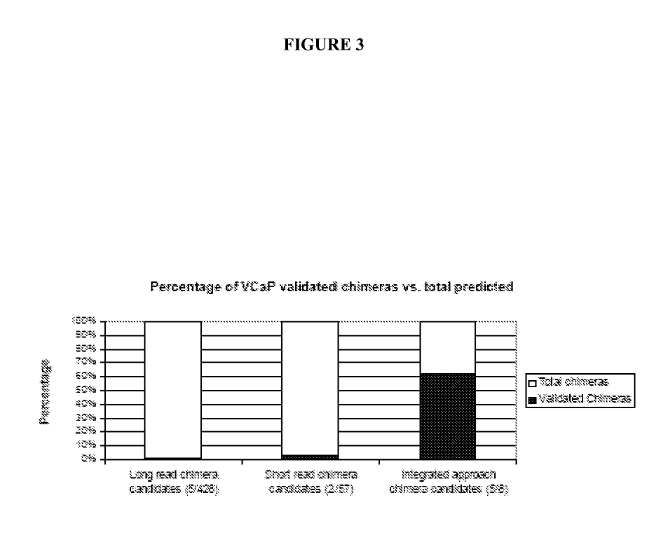

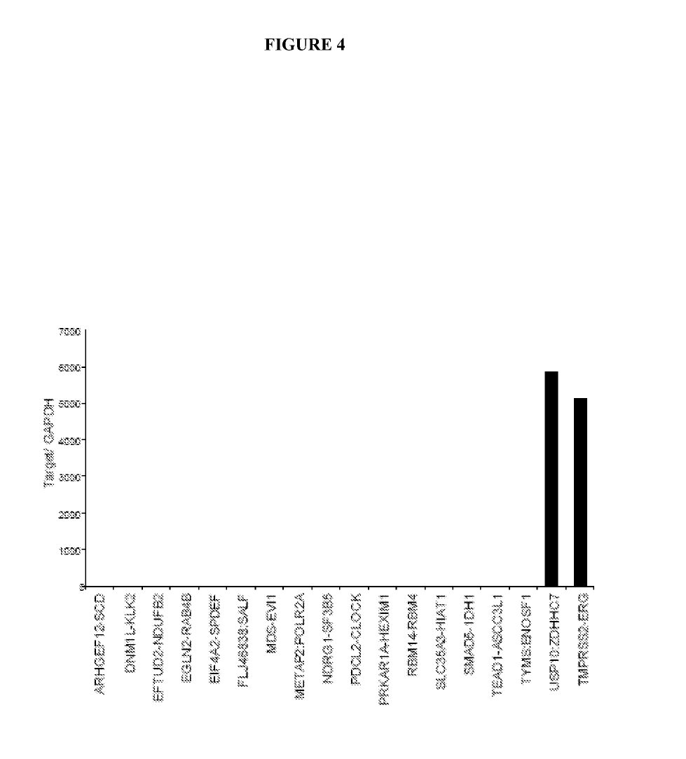

Next, using the Illumina Genome Analyzer over 50 million short transcriptome sequence reads were obtained from VCaP, LNCaP and RWPE cDNA libraries (TABLE 3). Focusing initially on VCaP cells, the TMPRSS2-ERG fusion was identified as one among 57 candidates, many of them likely false positives. To overcome the problem of false positives, lack of depth in long reads, and difficulty in mapping partially aligning short reads, integration of the long and short read sequence data was considered. Following this strategy, the single long read chimeric sequence spanning TMPRSS2-ERG junction from VCaP transcriptome sequence was found, buttressed by 21 short reads (FIG. 2) and existing as one of only eight chimeras nominated, overall. Thus, using the integrative approach the total number of false candidates was reduced and the proportion of experimentally validated candidates increased dramatically (FIG. 3). Extending the integrative analysis to LNCaP and RWPE sequences provided a total of fifteen chimeric transcripts, of which ten could be experimentally confirmed (TABLE 4). To ensure that the integration strategy filtered out only false positives and not valid chimeras, a panel of 16 long read chimera candidates that were eliminated upon integration was tested. None of them confirmed a fusion transcript by qRT-PCR (FIG. 4).

In order to systematically leverage the collective coverage provided by the two sequencing platforms, and to prioritize the candidates, a scoring function was formulated. Scores were obtained by multiplying the number of chimeric reads derived from either method (TABLE 4). Further, these chimeras were categorized as intra- or interchromosomal, based on their location on the same or different chromosomes, respectively. The latter represent bona fide gene fusions as do intra-chromosomal chimeras aligning to non-adjacent transcripts; intra chromosomal chimeras between neighboring genes are classified as (read-throughs) TMPRSS2-ERG was the top ranking gene fusion sequence, second only to a read-through chimera ZNF577-ZNF649.

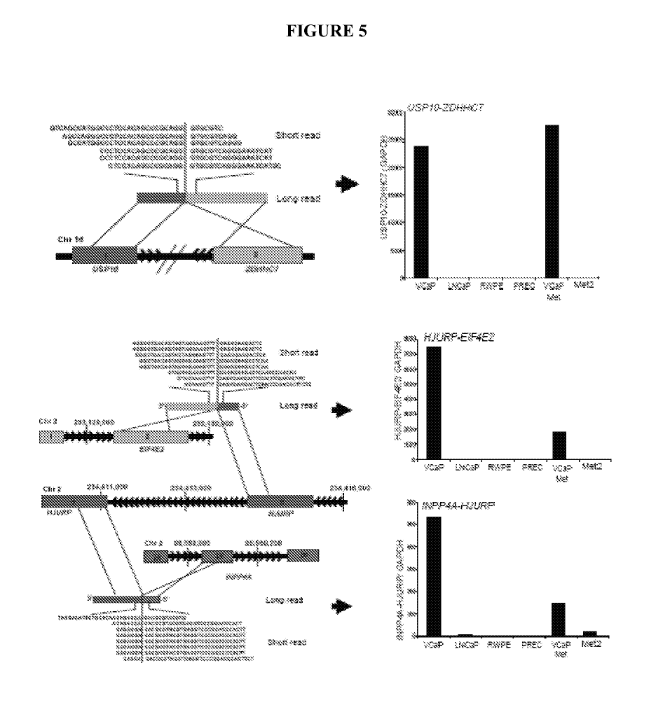

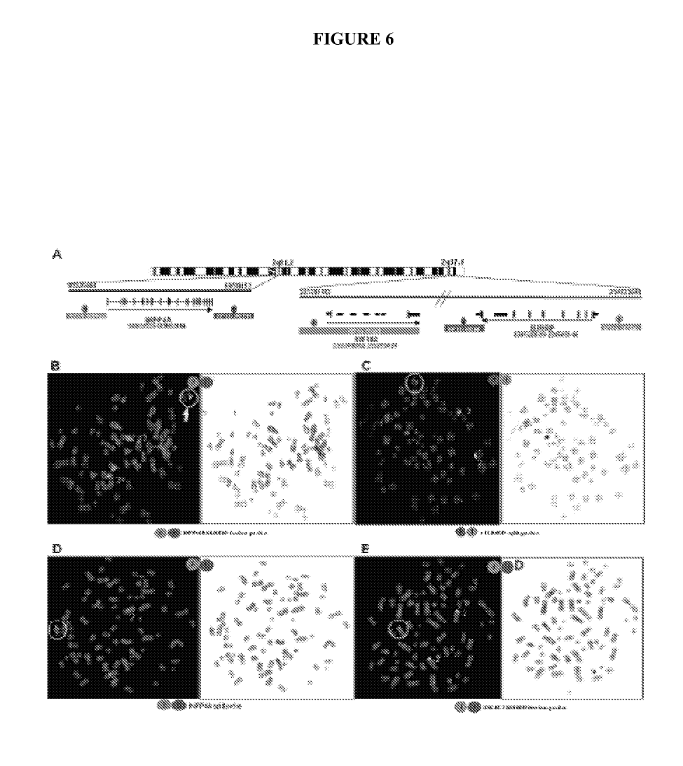

In addition to TMPRSS2-ERG, several new gene fusions were identified in VCaP. One such fusion was between exon 1 of USP10, with exon 3 of ZDHHC7, both genes located on chromosome 16, approximately 200 kb apart, in opposite orientation (FIG. 5). Furthermore, two separate fusions involving the gene HJURP on chromosome 2 were identified. A fusion between exon 2 of EIF4E2 with exon 8 of HJURP generated the fusion transcript EIF4E2-HJURP and a fusion between exon 9 of HJURP with exon 25 of INPP4A yielded HJURP-INPP4A (FIG. 5, FIG. 6).

This unexpected and complex intra-chromosomal rearrangement involving HJURP in VCaP was explored further. The fact that both exon 8 and 9 of HJURP fuse to different genes indicates a breakpoint resides within the intron (FIG. 5). Both of these gene fusions were confirmed by qRT-PCR in VCaP and VCaP-Met, and were found to be absent in other samples tested. This complex intrachromosomal rearrangement was also confirmed by FISH analysis. HJURP has been shown to be associated with genomic instability and immortality in cancer cells (Kato et al., Cancer Res. 67:8544 [2007]), while INPP4A encodes one of the enzymes involved in phosphatidylinositol signaling pathways and EIF4E2 is a eukaryotic translation initiation factor (Greenman et al., Nature 446:153 [2007]).

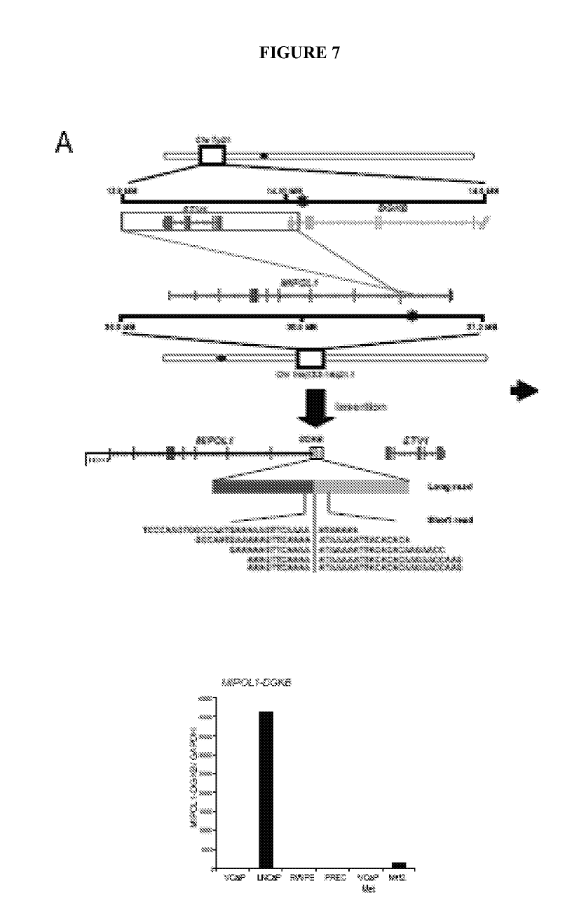

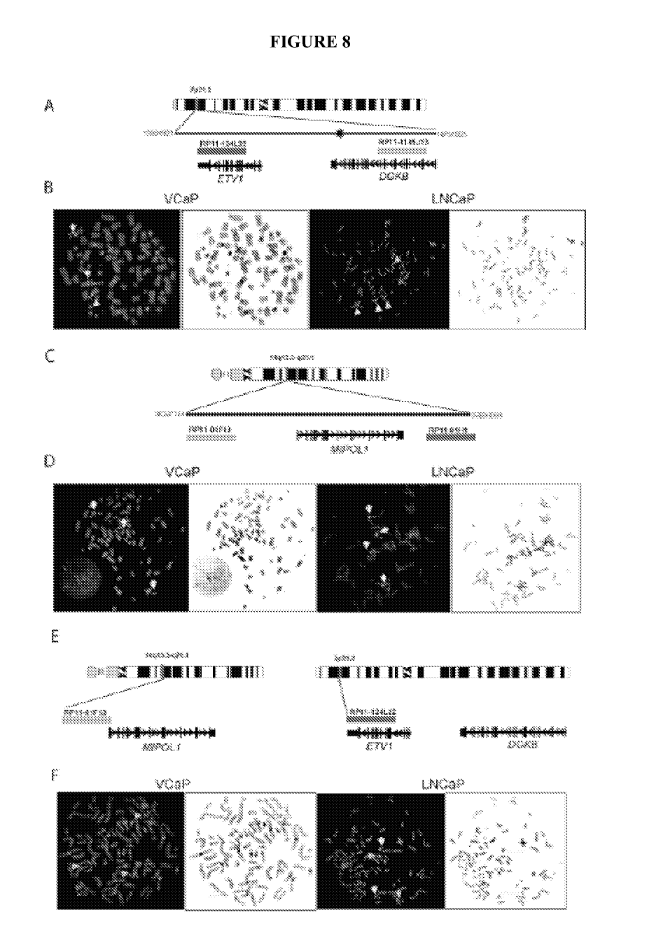

Interestingly, based on whole transcriptome sequencing, the highest ranked LNCaP gene fusion was between exon 11 of MIPOL1 on chromosome 14 with the last exon of DGKB on chromosome 7; confirmed by qRT-PCR and FISH (FIG. 7, FIG. 8). It was recently demonstrated that over-expression of ETV1, a member of the oncogenic ETS transcription factor family, plays a role in tumor progression in LNCaP cells3. While an understanding of the mechanism is not necessary to practice the present invention and while the present invention is not limited to any particular mechanism of action, the mechanism of ETV1 over-expression was attributed to a cryptic insertion of approximately 280 Kb encompassing the ETV1 gene into an intronic region of MIPOL1. Thus, while previous studies suggested that ETV1 was rearranged without evidence of an ETV1 fusion transcript, herein is shown evidence of the generation of a surrogate fusion of MIPOL1 to DGKB, which appears to be indicative of an ETV1 chromosomal aberration.

In addition to gene fusions, several transcript chimeras were identified between neighboring genes, referred to as read-through events. Overall, the read-through events appear to be more broadly expressed across both malignant and benign samples whereas the gene fusions were cancer cell specific (FIG. 9). For instance, a chimera between exon 2 of C19 orf25 with an intron of the neighboring gene APC2 in LNCaP cells (FIG. 9). Experimental validation demonstrated a lower expression level of C19orf25-APC2(intron) than observed for gene fusions and weak expression in multiple cell lines suggesting they are more broadly expressed. A similar pattern was observed for WDR55-DND1 (FIG. 9), MBTPS2-YY2 (FIG. 9), and ZNF649-ZNF577 (FIG. 9).

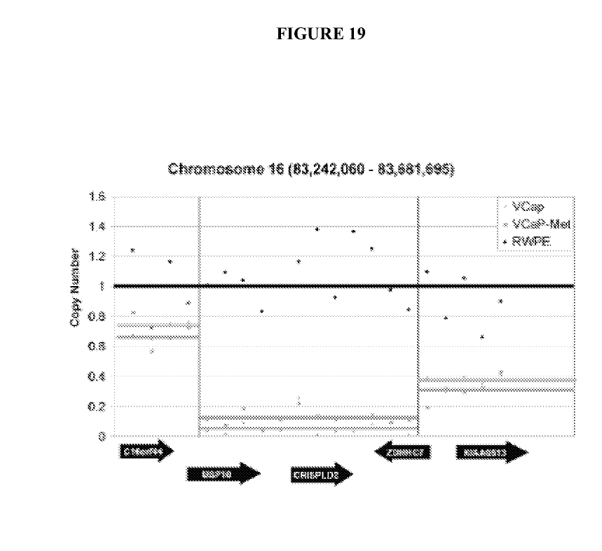

Many studies utilize genomic information for mining gene fusion candidates (Campbell et al., Nature Genet. 40:722 [2008]; Bashir et al., PLoS Comput. Biol. 4:e1000051 [2008]). Therefore, it was desirable to determine whether transcriptome data detects chimeras that would not be apparent from genomic DNA analysis. To do so, unbalanced genomic copy number change data from array comparative genomic hybridization of matched samples was integrated and genomic aberrations were monitored within gene fusion candidates. This revealed breakpoints in genes involved in two gene fusion candidates, USP10-ZDHHC7, and MIPOL1-DGKB (TABLE 4). More specifically, a homozygous deletion was observed to span the region between USP10-ZDHHC7 in VCaP cells as well as in the parental metastatic prostate cancer tissue from which VCaP is derived (VCaP-Met) but not in the normal prostate cell line RWPE (FIG. 19). While an understanding of the mechanism is not necessary to practice the present invention and while the present invention is not limited to any particular mechanism of action, taken together, this indicates that a deletion coupled with a complex rearrangement may have led to the USP10-ZDHHC7 fusion. qRT-PCR based evaluation confirmed this fusion to be specific to VCaP and its parental tissue, VCaP-Met, and not in LNCaP, RWPE, PREC, or metastatic prostate cancer tissue (Met 2) (FIG. 5). In LNCaP cells, for the MIPOL1-DGKB fusion a breakpoint was found only in DGKB but not in MIPOL1. Furthermore, absence of breakpoints in all other fusion chimeras examined indicates that the majority of fusion gene candidates identified by sequencing would not have been discovered by mining genomic copy number aberration data. Moreover, while only a subset of genomic rearrangements potentially represent functional gene fusions, most chimeric transcripts signify productive fusions, with likely roles in the biology of cells they are found in.

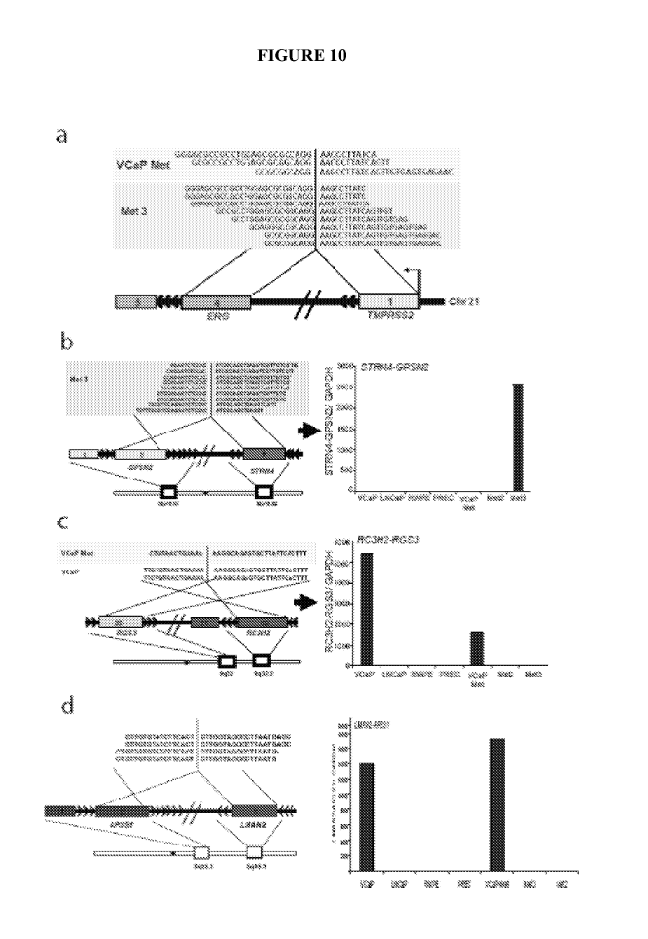

Next, this methodology was extended to tumor samples that represent the malignant cells often admixed with benign epithelia, stromal, lymphocytic, and vascular cells. Transcriptome sequencing was performed using two TMPRSS2-ERG gene fusion positive metastatic prostate cancer tissues, VCaP-Met (from which the VCaP cell line is derived) and Met 3, and one ERG negative metastatic prostate tissue, Met 4. In addition to the TMPRSS2-ERG fusion sequences detected in both VCaP-Met and Met 3 tissues, three novel gene fusions were identified (FIG. 10). One chimeric transcript from Met 3 involves exon 9 of STRN4 with exon 2 of GPSN2 (FIG. 10). GPSN2 belongs to the steroid 5-alpha reductase family, the enzyme that converts testosterone to dihydrotestosterone (DHT), the key hormone that mediates androgen response in prostate tissues. DHT is known to be highly expressed in prostate cancer, and is a therapeutic target. DHT, like its synthetic analog R1881, has been shown to induce TMPRSS2-ERG expression as well as PSA2. Additionally, exon 10 of RC3H2 was found to be fused to exon 20 of RGS3 in the VCaP-Met (and VCaP cells) (FIG. 10). Another novel gene fusion was between exon 1 of LMAN2 and exon 2 of AP3S1 (FIG. 10).

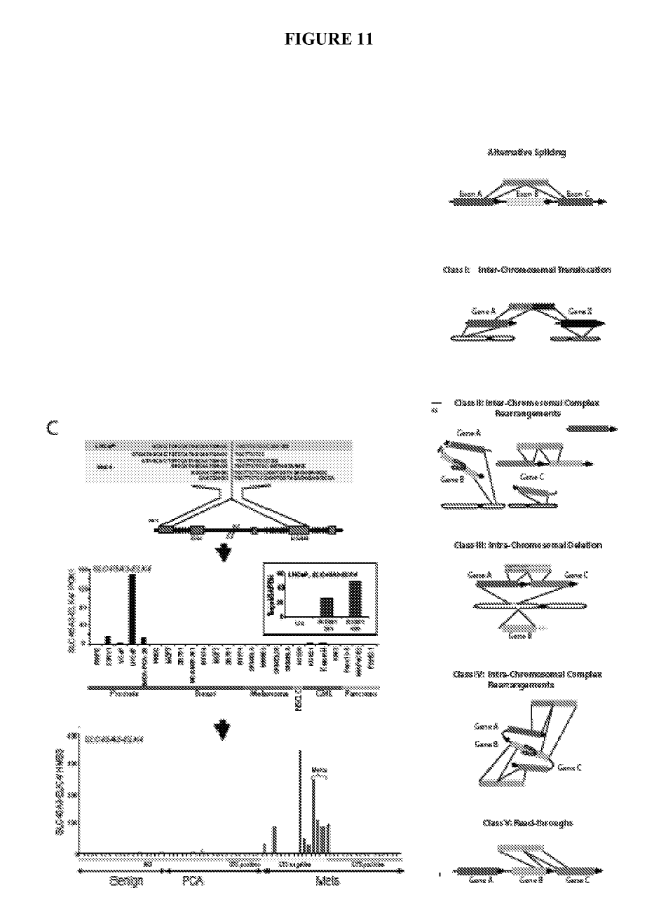

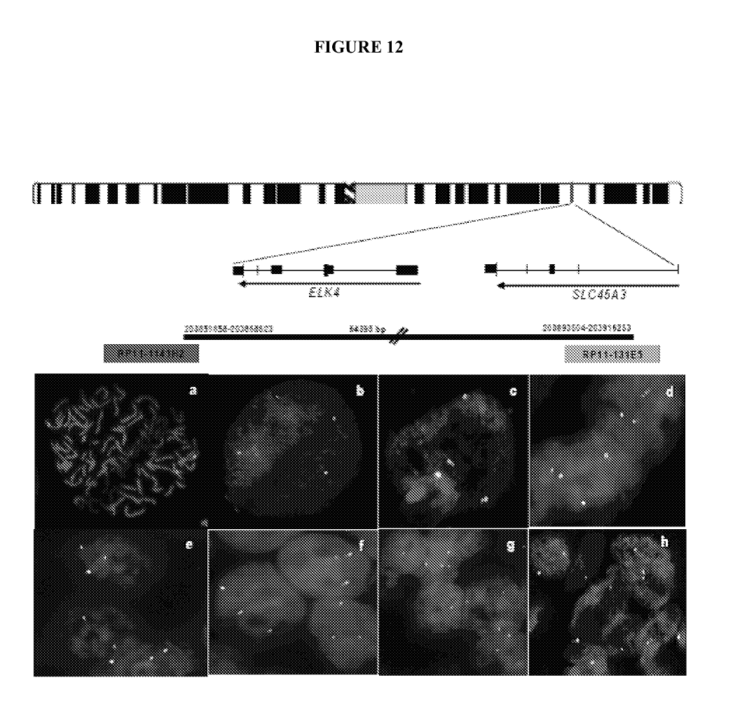

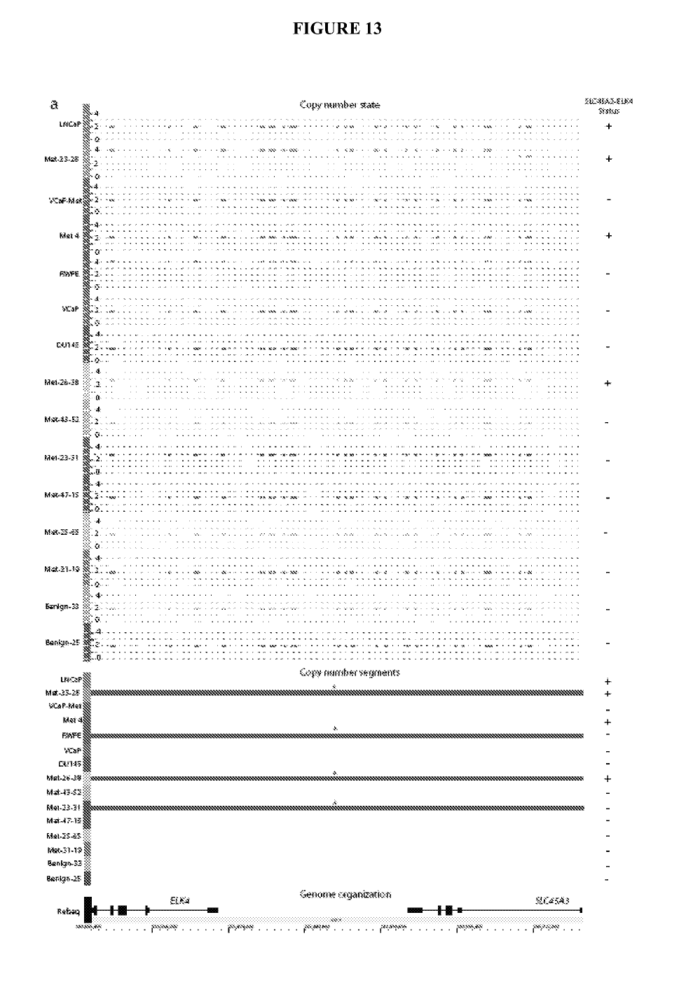

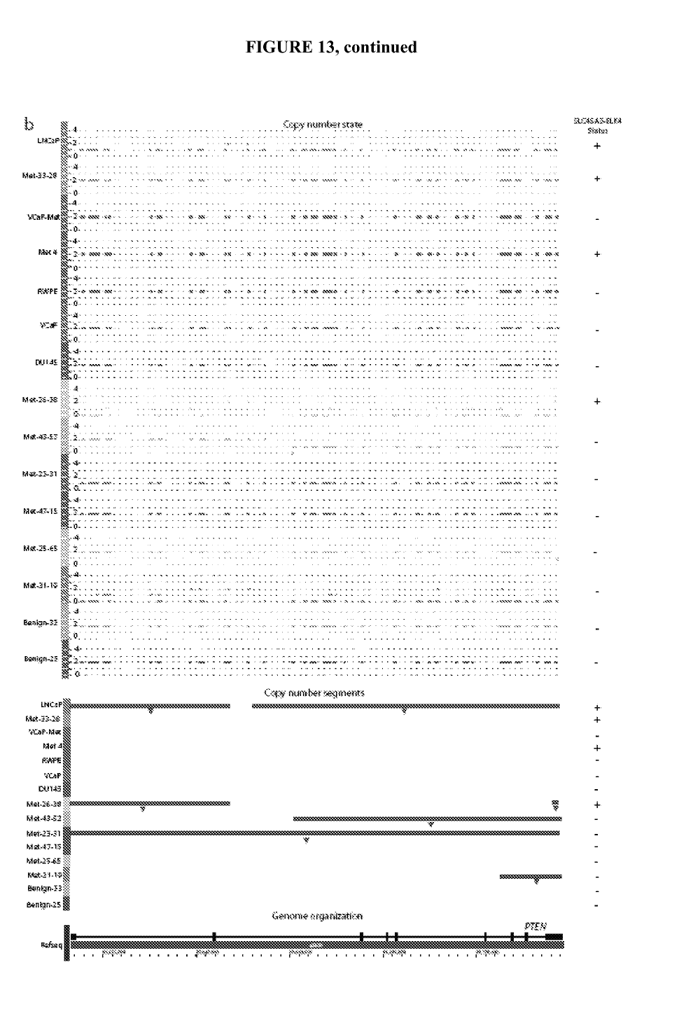

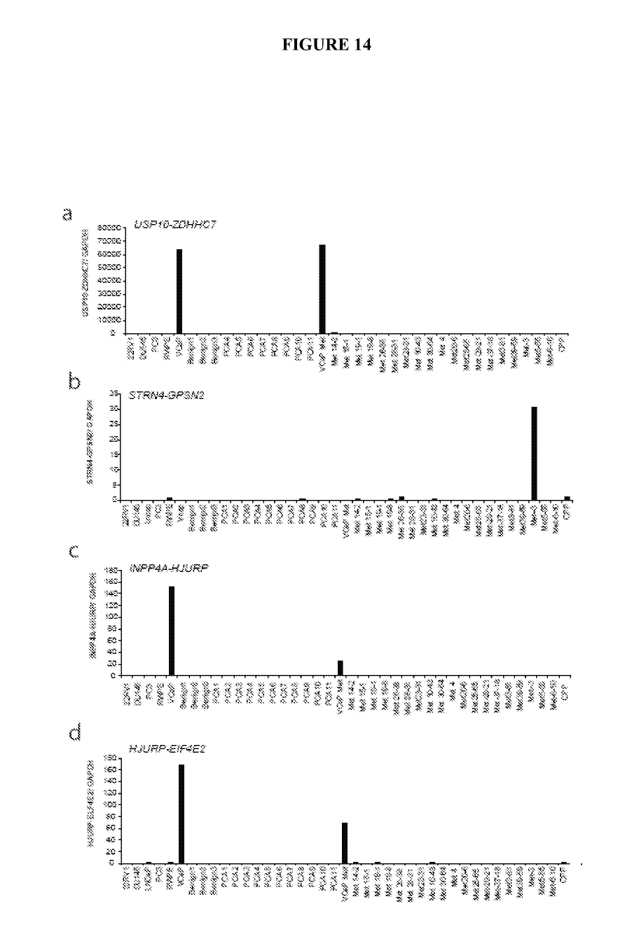

One read-through chimera, SLC45A3-ELK4, between the fourth exon of SLC45A3 with exon 2 of ELK4, a member of the ETS transcription factor family, was identified in metastatic prostate cancer, Met 4, and the LNCaP cell line indicating recurrence (FIG. 11). Taqman qRT-PCR assay for this fusion carried out in a panel of cell lines revealed high level of expression in LNCaP cells and much lower levels in other prostate cancer cell lines including 22Rv1, VCaP, and MDA-PCA-2B. Benign prostate epithelial cells, PREC and RWPE and non-prostate cell lines including breast, melanoma, lung, CML, and pancreatic cancer cell lines were negative for this fusion (FIG. 11). SLC45A3 has been earlier reported to be fused to ETV1 in a prostate cancer sample3, and notably, it is a prostate specific, androgen responsive gene. The fusion transcript SLC45A3-ELK4 was also found to be induced by the synthetic androgen R1881 (FIG. 11). Further, a panel of prostate tissues was interrogated for this fusion, and it was found to be expressed in seven out of twenty metastatic prostate cancer tissues examined (FIG. 11). Six of those seven positive cases have been identified as negative for ETS genes ERG, ETV1, ETV4, and ETV5 in previous work, based on a FISH screen (Han et al., Cancer Res. 68:7629 [2008]). One TMPRSS2-ETV1 positive metastatic prostate cancer sample was also found to be positive for SLC45A3-ELK4 (similar to LNCaP, which is also ETV1 positive (Tomlins et al., Nature 448:595 [2007])). Unlike the previous ETS gene fusions identified, SLC45A3-ELK4 is a read-through event between adjacent genes and does not harbor detectable alterations at the DNA level by FISH (FIG. 12), array CGH (data not shown) or high-density SNP arrays (FIG. 13). As LNCaP and Met 4 harbor genomic aberrations of ETV1, and express high levels of the SLC45A3-ELK4 chimeric transcript, this suggests that ETV1 and ELK4 may cooperate to drive prostate carcinogenesis in those tumors. While an understanding of the mechanism is not necessary to practice the present invention and while the present invention is not limited to any particular mechanism of action, SLC45A3-ELK4 may represent the first description of a recurrent RNA chimeric transcript specific to cancer that does not have a detectable DNA aberration. Overall, SLC45A3-ELK4 appears to be the only recurrent chimeric transcript identified in the transcriptome sequencing study, as other gene fusions tested in a panel of prostate cancer samples, appear to be restricted to the sample in which they were identified (at least in the limited number of samples analyzed) and thus may represent rare or private mutations (FIG. 14).

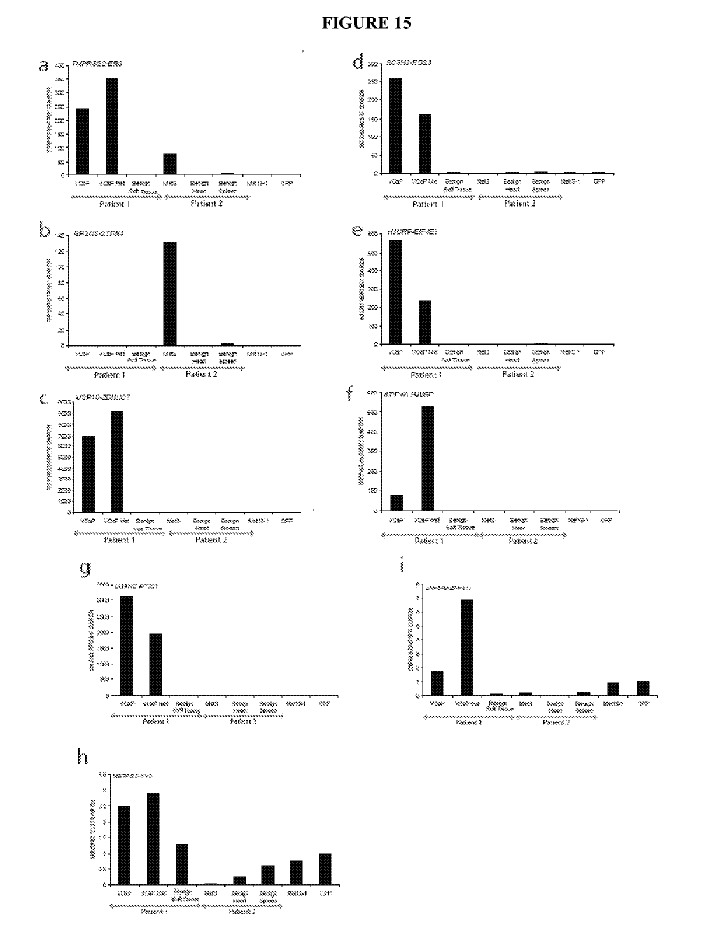

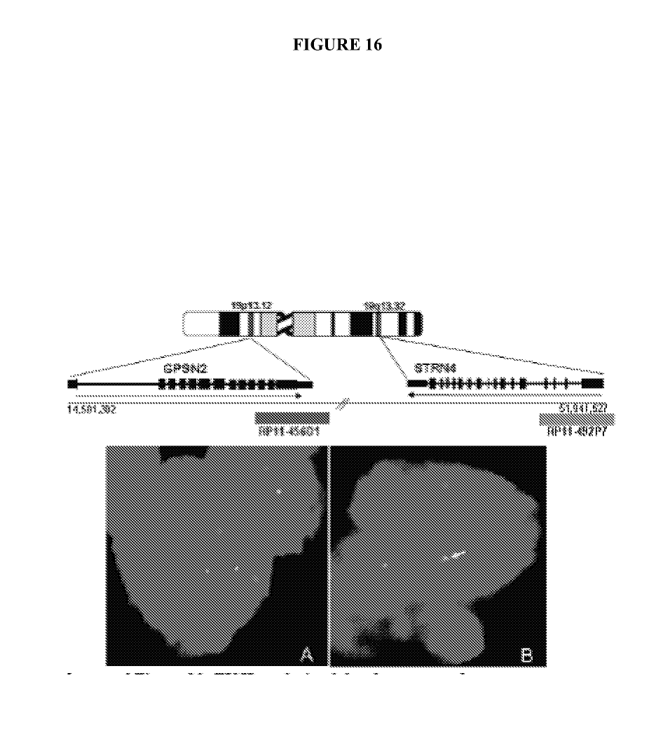

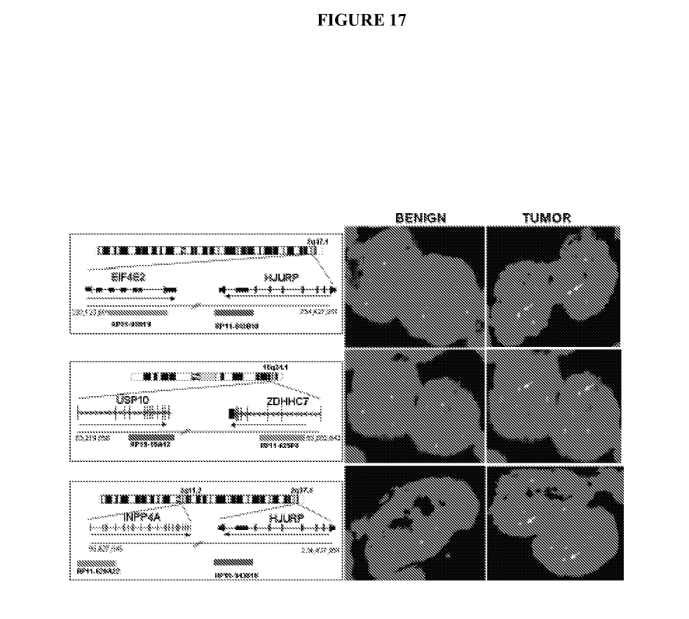

Next novel gene fusions identified in this study were tested to determine whether they represent acquired somatic mutations or simply, germline variations. Based on qPCR (FIG. 15) and FISH (FIG. 16, FIG. 17) assessment of a representative set of fusion genes on patient matched germline tissues, the chimeras were found to be restricted to the cancer tissues. Further, the 29 genes involved in the novel gene fusions were interrogated in the Database of Genomic Variants. Only 8 of them were found to have previously reported copy number variations (CNVs) (TABLE 5), but matched aCGH data did not reveal any copy number variation in those genes (TABLE 6), indicating that the samples analyzed did not harbor CNVs common to the human population.

Based on the gene fusions characterized (TABLE 7), a chimera classification system was proposed (FIG. 11). Inter-chromosomal translocation (Class I) involves fusion between two genes on different chromosomes (for example, BCR ABL1). Inter-chromosomal complex rearrangements (Class II) where two genes from different chromosomes fuse together while a third gene follows along and becomes activated (MIPOL1-DGKB). Intra-chromosomal deletion (Class III) results when deletion of a genomic region fuses the flanking genes (TMPRSS2-ERG). Intra-chromosomal complex rearrangements (Class IV) involve a breakpoint in one gene fusing with multiple regions (HJURP-EIF4E2, and INPP4-HJURP) and Read-through chimeras (Class V) include chimeric transcripts between neighboring genes (ZNF649-ZNF577).

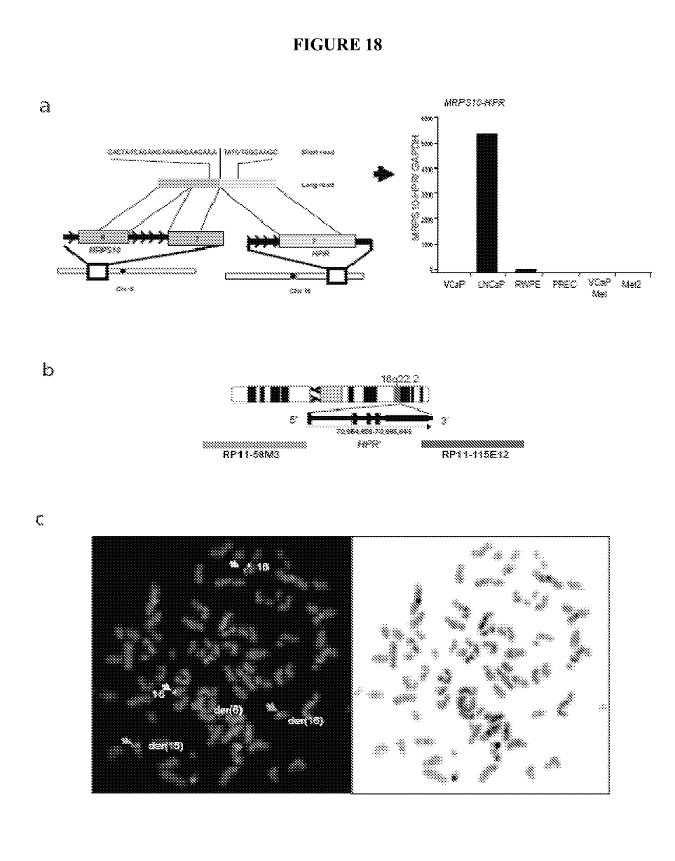

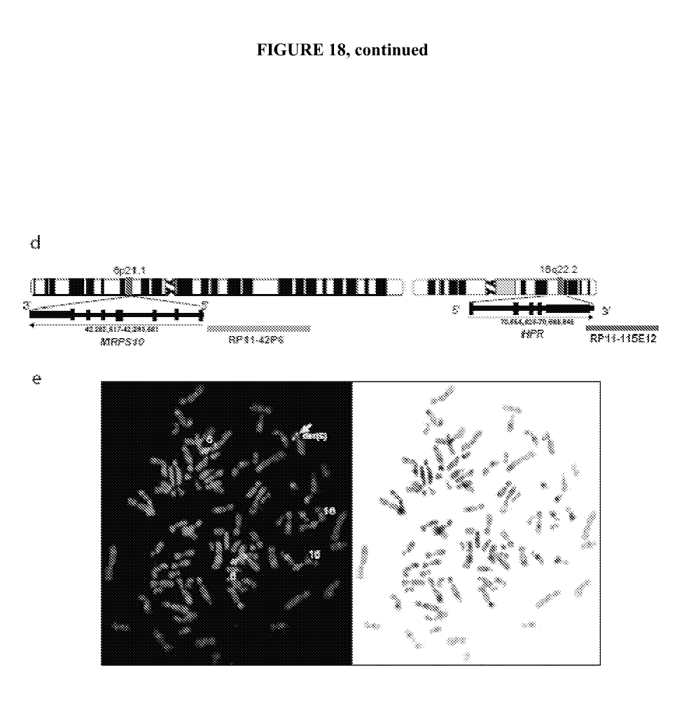

The top gene fusion nomination in LNCaP cells involved the fusion of MIPOL1-DGKB. This gene fusion may represent a harbinger of ETV1 cryptic rearrangement, a putative driver mutation in the LNCaP prostate cancer cell line. Moreover, it was observed that the LNCaP cells harbor multiple fusions, similar to observations in VCaP. One of the validated examples is the fusion between exon 7 of MRPS10 from chromosome 6 with exon 7 of HPR of chromosome 16 (FIG. 18). MRPS10-HPR was confirmed by FISH and validated by qRT-PCR in LNCaP, but not observed in VCaP, VCaP-Met, RWPE, PREC, or Met 2 (FIG. 18).

TABLE 1
Summary of normalized and non-normalized VCaP 454 libraries

| Sample | Normalized VCaP | Non-normalized VCaP |
|---|---|---|
| Subtracted | Yes | No |
| Total Reads | 575985 | 551780 |
| Average length | 218.9 | 226.5 |
| Genes* | 2687 | 2857 |
| Reads/Gene | 214.35 | 193.14 |
| Chimeras | 118 | 428 |
| Reads/chimera | 4881.3 | 1289.3 |

*A read must be a best hit to the gene with greater than 90% alignment

TABLE 8
Primer sequences used for confirming
fusion genes by qRT-PCR.

| Fusion | | |
|---|---|---|
| Gene | Primer Sequence (5′-3′) | SEQ ID NO. |
| ARHGEF12- | GCTAAGGAAAGGGTGGGATG | SEQ ID NO. 7 |
| SCD-F | | |
| ARHGEF12- | TTGTGTTTGTTCATAATAAAAAG | SEQ ID NO. 8 |
| SCD-R | TGAA | |
| BCR-ABL | GAGTCTCCGGGGCTCTATGG | SEQ ID NO. 9 |
| (b3a2)-F | | |
| BCR-ABL | GCCGCTGAAGGGCTTTTGAA | SEQ ID NO. 10 |
| (b3a2)-F | | |
| DNM1L- | GGATCCTCCCCTTCTTTCTG | SEQ ID NO. 11 |
| KLK2-F | | |
| DNM1L- | CAAAACTTGCTAGTTACTGCCTACC | SEQ ID NO. 12 |
| KLK2-R | | |
| EFTUD2- | CCCAGCACCTCTTCTGAGTC | SEQ ID NO. 13 |
| NDUFB2-F | | |
| EFTUD2- | AGAGAGGGGTGTAGGCATCA | SEQ ID NO. 14 |
| NDUFB2-R | | |
| EGLN2- | GGATTGTCAACGTGCCCTAC | SEQ ID NO. 15 |
| RAB4B-F | | |
| EGLN2- | GAGCTAGACCCGGAGAGGAT | SEQ ID NO. 16 |
| RAB4B-R | | |
| EIF4A2- | GTGCACGAACTGGTAGACGA | SEQ ID NO. 17 |
| SPDEF-F | | |
| EIF4A2- | GGCAGAAAGCAACACAACCT | SEQ ID NO. 18 |
| SPDEF-R | | |
| LMAN2- | ACTGACGGCAACAGTGAACA | SEQ ID NO. 19 |
| AP3S1-F | | |
| LMAN2- | TGGAAAGTCTCCCTGATGATTT | SEQ ID NO. 20 |
| AP3S1-R | | |
| MDSI- | ATGCAACAAGGTTGTGCTGA | SEQ ID NO. 21 |
| EVI1-F | | |
| MDSI- | CAAACCTGAAAGACCCCAGT | SEQ ID NO. 22 |
| EVI1-R | | |
| MIA2- | AGCCGACTCCTAACCGATCT | SEQ ID NO. 23 |
| CTAGE5-F | | |
| MIA2- | TGAATTCTGCATTTTCACCAA | SEQ ID NO. 24 |
| CTAGE5-R | | |
| MIPOL1- | CAGAGCGAGCAAATATGGAA | SEQ ID NO. 25 |
| DGKB-F | | |
| MIPOL1- | CTTGCTTCGGTTTCTTGTCC | SEQ ID NO. 26 |
| DGKB-R | | |
| NDRG1- | CAAAAACGAGACGCCAAATC | SEQ ID NO. 27 |
| SF3B5-F | | |
| NDRG1- | CAAAAACAAGACGCGTAGCA | SEQ ID NO. 28 |
| SF3B5-R | | |
| PDCL2- | GAAGCGGTTACAGGAATGGA | SEQ ID NO. 29 |
| CLOCK-F | | |
| PDCL2- | TTCTGAGCTCCAGCAGCTTT | SEQ ID NO. 30 |
| CLOCK-R | | |
| PRKAR1A- | GAACTGAGCAGAGCAGAGCA | SEQ ID NO. 31 |
| HEXIM1-F | | |
| PRKAR1A- | CATTTGGCATTAACAAAGATCAA | SEQ ID NO. 32 |
| HEXIM1-R | | |
| RBM14- | GTGTGACGTGGTGAAAGGTG | SEQ ID NO. 33 |
| RBM4-F | | |
| RBM14- | AAATGGGCAGGAGAGGAAAG | SEQ ID NO. 34 |
| RBM4-R | | |
| RC3H2- | GCTAATGGTCAGAATGCTGCT | SEQ ID NO. 35 |
| RGS3-F | | |
| RC3H2- | CTTCTTCTGCTCCTGCGAGT | SEQ ID NO. 36 |
| RGS3-R | | |
| SLC35A3- | GCTGTCAATAGTCCCCAAGC | SEQ ID NO. 37 |
| HIAT1-F | | |
| SLC35A3- | GGATTTGCAACCTCTTTATCG | SEQ ID NO. 38 |
| HIAT1-R | | |
| SMAD5- | TTTGGGGATAAGGGAAAAGG | SEQ ID NO. 39 |
| IDH1-F | | |
| SMAD5- | GCTTTGCTCTGTGGGCTAAC | SEQ ID NO. 40 |
| IDH1-R | | |
| STRN4- | CTGGGGGACTTGGCAGAT | SEQ ID NO. 41 |
| GPSN2-F | | |
| STRN4- | TCCAAGAAACACAGCTTCTCC | SEQ ID NO. 42 |
| GPSN2-R | | |
| TEAD1- | GGCTCAGGTTGTGGTAGAGG | SEQ ID NO. 43 |
| ASCC3L1-F | | |
| TEAD1- | TTGAGCCTGTCCTGGAACTT | SEQ ID NO. 44 |
| ASCC3L1-R | | |
| TMPRSS2- | GGAGTAGGCGCGAGCTAAG | SEQ ID NO. 45 |
| ERG-F | | |
| TMPRSS2- | GTCCATAGTCGCTGGAGGAG | SEQ ID NO. 46 |
| ERG-R | | |
| USP10- | CGGAGTCCCAATGAAACG | SEQ ID NO. 47 |
| ZDHHC7-F | | |
| USP10- | GAGGAGGAGGACGATGAAGA | SEQ ID NO. 48 |
| ZDHHC7-R | | |
| ZNF577- | CCTTCCCAGAAGTGGTGGT | SEQ ID NO. 49 |
| ZNF649-F | | |
| ZNF577- | CACACGGGAGAGAGACCCTA | SEQ ID NO. 50 |
| ZNF649-R | | |
| MRPS10- | GATTCTTGGGCTTCCCACAT | SEQ ID NO. 51 |
| HPR-F | | |
| MRPS10- | CAAAGACACAATTAGAACAGTTACCA | SEQ ID NO. 51 |
| HPR-R | | |
| SLC45A3- | GCAGATCCTGCCCTACACAC | SEQ ID NO. 53 |
| ELK4-F | | |
| SLC45A3- | AGCTGAAGAAGGAACTGCCA | SEQ ID NO. 54 |
| ELK4-R | | |

TABLE 9
Sequences of chimeric transcripts,
with GenBank accession numbers.
Fusion junction is denoted by ‘*’.

>TMPRSS2-ERG FJ423744
(SEQ ID NO. 55)
GGAGTAGGCGCGAGCTAAGCAGGAGGCGGAGGCGGAGGCGGAGGGC
GAGGGGCGGGGAGCGCCGCCTGGAGCGCGGCAG*GAAGCCTTATCA
GTTGTGAGTGAGGACCAGTCGTTGTTTGAGTGTGCCTACGGAACGC
CACACCTGGCTAAGACAGAGATGACCGCGTCCTCCTCCAGCGACTA
TGGACAGACTTCCAAGATGAGCCCACGCGTCCCTCAGCAGGATTGG
CTGTCT
>INPP4A-HJURP FJ423742
(SEQ ID NO. 56)
AGGTCTCAAGAATCAAAAACAAAACAAAAATACAAACAGAGAGCAA
GTGGGAAGATAAATAACACTCCGAAATAACCTAGCTACACACTTTT
AGTTTCCAATTTTTCTTAGCATGAAATCACTTTTCTCTTCCATCCT
GTAAGACGTGTTCTCTCCT*CTGCGCATGCACTCCAGGGCCTGGGT
GAAGACCTGCGGGGCCATGCCATGCTCGTGTTGCAGGATCAGGCAC
TGCTCCAGTGTCACCG
>ZNF649-ZNF577 FJ423743
(SEQ ID NO. 57)
GGGGCTAGCAACTCTAGTATGTTTTCTCTCTTCTGTCTATTCTGGG
CCTTCCCAGAAGTGGTGGTCAGGTATCATCTCAGGTCAAGCTACCA
CTGGAAATGATGATCTTCCCCAGCCTGGAAGCTCCTTCTTCCATTA
CTGAAAATGTCTTGTTCCTATAGGCCAGAAC*ACTCATCACAGCCA
TAGGGTCTCTCTCCCGTGTGAGTTCTGTGATGTACAATGAGCATTG
>USP10-ZDHHC7 FJ423745
(SEQ ID NO. 58)
ACGCGGGGGAAGCAGCGTGAGCAGCCGGAGGATCGCGGAGTCCCAA
TGAAACGGGCAGCCATGGCCCTCCACAGCCCGCAG*GGTGCGTCAG
GGAAATCATGCAGCCATCAGGACACAGGCTCCGGGACGTCGAGCAC
CATCCTCTCCTGGCTGAAAATGACAACTATGACTCTTCATCGTCCT
CCTCCTCCGAGGCTGACGTGGCTGACCGGGTCTGGTTCATCCGTGA
CGG
>HJURP-EIF4E2 FJ423746
(SEQ ID NO. 59)
CGATTCTTGTCTCGTTCCGTTTTTTCCTTCTCACCATCTTTCTGTG
TGCTGTTTTCTTCATTCTGATCATGGTCCCCACTGTCATCATCTTT
CAAA*CTCTCTTCTGAGTTGGGCTGTGAAGAGCTGCCCTGGTCTCC
CGGTCTGACGGTGTTGTCCACCCCATCTGAGGCACCCAGGGAATTG
CCCTGGCGTCCGGAGCCCGTGGGTTCTGATAGCCTGGGTCTTTTTG
CAGGGAACTGATGGT
>MIPOLl-DGKE FJ423747
(SEQ ID NO. 60)
ACAGAGAGAACATTGTTTCCATCACTCAACAACAAAATGAGGAACT
GGCTACTCAACTGCAACAAGCTCTGACAGAGCGAGCAAATATGGAA
TTACAACTTCAACATGCCAGAGAGGCCTCCCAAGTGGCCAATGAAA
AAGTTCAAAA*ATAAAAATTACACACAAGAACCAAGCCCCAATGCT
GATGGGCCCGCCTCCAAAAACCGGTTTATTCTGCTCCCTCGTCAAA
AGGACAAGAAACCGAAGCAAGGAATAA
>MRPS10-HPR FJ423748
((SEQ ID NO. 61)
GTCACTGGGTTTGCCGGATTCTTGGGCTTCCCACATA*TTTCTTCT
TTTTCTTCTGATAGTGTTTCCCAGATTGGCTCCTTGATGTGTTCTG
GTAACTGTTCTAATTGTGTCTTTGTTACTTCCATGGCAACCCCTTC
AGGTAAGTTTCA
>WDR55-DND1 FJ423749
(SEQ ID NO. 62)
CGCAAAAAAAAGGGAGGACCACTGCGGGCTCTGAGCAGCAAGACTT
GGAGCACCGATGACTTCTTCGCAGGACTGAGGGAAGAGGGAGAAGA
CTCCATGGCTCAGGAAGAAAAGGAGGAGACTGGGGATGACAATGAC
TGAAGGAATGAATTGAATCTTGAGACGGGTCCTCACCAGGGTGCCT
GTGGAGAAAGAATGGAGTCACTGTTTAACCATGGTACCTGCCTCAG
CCCCAGCAGACCACAGGAGGTTCGG
>C19orf25-APC2 (Intron) FJ423750
(SEQ ID NO. 63)
GAATCGGAAGTGGCTGCGTCGTCGACGCTGGGCTTTCGGGTCCCGC
GCCCAGAGATGGGCTCCAAGGCAAAGAAGCGCGTGCTGCTGCCCCA
CCCGCCCAGCGCCCCCCACGGGTGGAGCAGATCCTGGAGGATGTGC
GGGGTGCGCCGGCAGAGGATCCAGTGTTCACCATCCTGGCCCCGGA
AG*GCTGGAGTGCAGTGGCGAGATCTCGACTCACTGCAGGCTCCGA
CTCCCCAGTTCAAGCGATT
>MBTPS2-YY2 FJ423751
(SEQ ID NO. 64)
TTGGGATTTTTCTCTTCATTATTTATCCCGGAGCATTTGTTGATCT
GTTCACCACTCATTTGCAACTTATATCGCCAGTCCAGCAGCAAGGA
TATTTTGTGCAG*CCATGGCCTCCAACGAAGATTTCTCCATCACAC
AAGACCTGGAGATCCCGGCAGATATTGTGGAGCTCCACGACATCAA
TGTGGAGCCCCTTCCTATGGAGGACATTCCGACGGAAAGCGTCCAG
TACG
>STRN4-GPSN2 FJ423752
(SEQ ID NO. 65)
CTGGGGGACTTGGCAGATCTCACCGTCACCAACGACAACGACCTCA
GCTGCGAT*GTGGAGATTCTGGACGCAAAGACAAGGGAGAAGCTGT
GTTTCTTGGA
>LMAN2-AP3S1 FJ423753
(SEQ ID NO. 66)
ACTGACGGCAACAGTGAACATCTCAAGCGGGAGCATTCGCTCATTA
AGCCCTACCAAG*AGTGAAGATACACAACAGCAAATCATCAGGGAG
ACTTTCCA
>RC3H2-RGS3 FJ423754
(SEQ ID NO. 67)
GCTAATGGTCAGAATGCTGCTGGGCCCTCTGCAGATTCTGTAACTG
AAAA*AAGGCAGAGTGCTTATTCACTTTGGAAGCGCACTCGCAGGA
GCAGAAGAAG
>SLC45A3-ELK4 FJ423755
(SEQ ID NO. 68)
GCTGAAGAAGGAACTGCCACAGGGTGATAGCACTGTCCATAGCAAT
GAG*CTGCTTCTCCCGGTGGTAGAGGGAGGCCAGTGTGTAGGGGAG
G

### Example 3

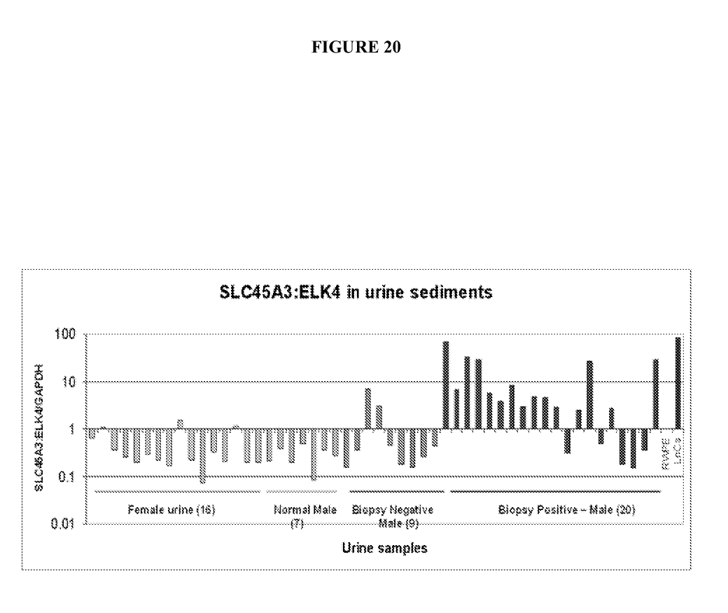

This Example describes the identification of SLC45A3:ELK4 mRNA in urine sediments. A TaqMan qRT-PCR assay using chimera-specific primers on urinary sediment samples was performed. Results are shown in FIG. 20.

### Example 4

Paired-End Gene Fusion Discovery Pipeline. Mate pair transcriptome reads were mapped to the human genome (hg18) and Refseq transcripts, allowing up to 2 mismatches, using Efficient Alignment of Nucleotide Databases (ELAND) pair within the Illumina Genome Analyzer Pipeline software. Illumina export output files wereparsed to categorize passing filtermatepairs as (i) mappingto the same transcript, (ii) ribosomal, (iii) mitochondrial, (iv) quality control, (v) chimera candidates, and (vi) nonmapping. Chimera candidates and nonmapping categories were used for gene fusion discovery. For the chimera candidates category, the following criteria were used: (i) mate pairs are of high mapping quality (best unique match across genome), (ii) best unique mate pairs do not have a more logical alternative combination (e.g., best mate pairs indicate an interchromosomal rearrangement, whereas the second best mapping for a mate resides results in the pair having the expected insert size), (iii) the sum of the distances between the most 5′ and 3′ mate on both partners of the gene fusion is <500 nt, and (iv) mate pairs supporting a chimera are nonredundant.

In addition to mining mate pairs encompassing a fusion boundary, the nonmapping category was mined for mate pairs that had 1 read mapping to a gene, whereas its corresponding read fails to align, because it spans the fusion boundary. First, the annotated transcript that the “mapping” mate pair aligned against was extracted, because this represents one of the potential partners involved in the gene fusion. The “nonmapping” mate pair was then aligned against all of the exon boundaries of the known gene partner to identify a perfect partial alignment. A partial alignment confirms that the nonmapping mate pairmaps to the expected gene partner while revealing the portion of the nonmapping mate pair, or overhang, aligning to the unknown partner. The overhang is then aligned against the exon boundaries of all known transcripts to identify the fusion partner. This is done using a Perl script that extracts all possible (UCSC) and Refseq exon boundaries looking for a single perfect best hit.

Mate pairs spanning the fusion boundary are merged with mate pairs encompassing the fusion boundary. At least 2 independent mate pairs were required to support a chimera nomination. This was achieved by (i) 2 or more nonredundant mate pairs spanning the fusion boundary, (ii) 2 or more nonredundant mate pairs encompassing a fusion boundary, or (iii) 1 or more mate pairs encompassing a fusion boundary and 1 or more mate pairs spanning the fusion boundary. All chimera nominations were normalized based on the cumulative number of mate pairs encompassing or spanning the fusion junction per million mate pairs passing filter. Chimeras were subsequently classified into inter and intrachromosomal gene fusions. The intrachromosomal gene fusions were further divided based on whether or not they were adjacent to one another.

RNA Chimera Analysis. Chimeras found from UHR, HBR, VCaP, and K562 were grouped based on whether they showed expression in all samples, “broadly expressed,” or a single sample, “restricted expression.” Because UHR is comprised of K562, chimeras found in only these 2 samples were also considered as restricted. Heatmap visualization was conducted by using TIGR's MultiExperiment Viewer (TMeV) version 4.0. RNA chimeras were given independent confirmation if one or more ESTs were found to overlap both genes involved in the predicted chimeric event.

Samples and cell lines. VCaP cell line was derived from a vertebral metastasis from a patient with hormone-refractory metastatic prostate cancer (Korenchuk et al. In Vivo 15:163 [2001]; herein incorporated by reference in its entirety). LNCaP or VCaP cells were starved in phenol red free media supplemented with charcoal-dextran filtered FBS and 5% penicillin/streptomycin for 48 h before the addition of 1 nM synthetic androgen (R1881) as indicated. RNA was then isolated using the microRNeasy kit (Qiagen) according to the manufacturer's instructions. Prostate tissues were obtained from the radical prostatectomy series at the University of Michigan and from the Rapid Autopsy Program (Rubin et al. Clin. Cancer Res. 6:1038 [2000]; herein incorporated by reference in its entirety), University of Michigan Prostate Cancer Specialized Program of Research Excellence (SPORE) Tissue Core. All samples were collected with informed consent of the patients and prior approval of the institutional review board. K562, SUP-B15, MEG-01, KU812, GDM-1, and Kasumi-4 cell lines were obtained from American Type Culture Collection (ATCC). UHR was obtained from Strategene. Human brain RNA (HBR) was obtained from Ambion.

Sequence datasets. Human genome build 18 (hg18) was used as a reference genome. All Refseq and University of California Santa Cruz (UCSC) transcripts were downloaded from the UCSC genome browser. Sequences of previously identified TMPRSS2-ERGa fusion transcript (GenBank accession no. DQ204772) and BCR-ABL1 fusion transcript (GenBank accession no. M30829) were used for reference. Previously validated prostate gene fusion chimaeras were extracted using GenBank accession nos. FJ423742-FJ423755.

Paired-end transcriptome sequencing using Illumina Genome Analyzer II. Messenger RNA (1 μg) was fragmented at 70° C. for 2 min in a fragmentation buffer (Applied Biosystems) and converted to single-stranded cDNA using SuperScript II reverse transcriptase (Invitrogen), followed by second-strand cDNA synthesis using Escherichia coli DNA polymerase I (Invitrogen). The doublestranded cDNA was further processed by Illumina mRNA sequencing Prep kit. Briefly, double-stranded cDNA was end repaired by using T4 DNA polymerase and T4 polynucleotide kinase, monoadenylated using a Klenow DNA polymerase I (3′ to 5′ exonucleotide activity), and ligated with adaptor oligo mix (Illumina) using T4 DNA ligase. The adaptor-ligated cDNA library was then fractioned on a 4% agarose gel, and a smear corresponding to approximately 300 nt was excised, purified, and PCR amplified (15 cycles) by Pfu polymerase (Stratagene). The PCR product was again size selected on a 4% agarose gel by cutting out the library smear at 300 base pairs. The library was then purified with the Qiaquick Minelute PCR Purification Kit (Qiagen) and quantified with the Agilent DNA 1000 kit on the Agilent 2100 Bioanalyzer following the manufacturer's instructions. Library (10 nM) was used to prepare flowcells with approximately 100,000-130,000 clusters per lane for analysis on the Illumina Genome Analyzer II.

Long transcriptome read gene fusion discovery. All 100-nt passing filter transcriptome reads generated from the Illumina sequencing platform were processed similar to the method described for detecting chimeras from 454 reads (Maher et al. Nature 458:97 [2009]; herein incorporated by reference in its entirety). All chimera nominations were normalized based on the total number reads spanning the fusion junction per million reads passing filter.

Comparison of single transcriptome reads with paired-end approach. As the 100-nt single transcriptome reads were aligned against only Refseq transcripts to identify chimeras spanning exon-exon boundaries, only those paired-end chimera nominations that had supporting evidence of an exon-exon fusion junction were used for comparison.

RNA chimera classification. Chimeras between adjacent genes were categorized based on their orientation to one another and whether they are overlapping. The categories are (i) readthroughs, adjacent genes in the same orientation, (ii) diverging genes, adjacent genes in opposite orientation whose 5′ sites are in close proximity, (iii) convergent genes, adjacent genes whose 3′ ends are in close proximity, and (iv) overlapping genes, adjacent genes who share common exons. Genes were defined as overlapping if they have even 1 nt overlapping.

Real-time PCR validation. Quantitative PCR was performed using Power SYBR Green Mastermix (Applied Biosystems) on an Applied Biosystems Step One Plus Real Time PCR System as described (Tomlins et al. Nature 448:595 [2007]; herein incorporated by reference in its entirety). All oligonucleotide primers were synthesized by Integrated DNA Technologies. GAPDH (Vandescompele et al. Genome Biol. 3:34 [2002]; herein incorporated by reference in its entirety) primer was as described. All assays were performed in duplicate or triplicate, and results were plotted as average fold change relative to GAPDH.

FISH. FISH hybridizations were performed on VCaP and prostate tumor samples. BAC clones were selected from the UCSC genome browser. After colony purification, midi prep DNA was prepared using QiagenTips-100 (Qiagen). DNA was labeled by nick translation labeling with biotin-16-dUTP and digoxigenin-11-dUTP (Roche). Probe DNA was precipitated and dissolved in hybridization mixture containing 50% formamide, 2×SSC, 10% dextran sulfate, and 1% Denhardts solution. Approximately 200 ng of labeled probes was hybridized to normal human chromosomes to confirm the map position of each BAC clone. FISH signals were obtained using anti digoxigenin-fluorescein and alexa fluor594 conjugate for green and red colors, respectively. Fluorescence images were captured using a high resolution CCD camera controlled by ISIS image processing software (Metasystems).

ChIP-Seq analysis. ChIP from the cultured cells was carried out as previously described (Yu et al. Cancer Cell 12:419 [2007]; herein incorporated by reference in its entirety), using antibodies against AR (no. 06-680; Millipore), ERG (no. sc354; Santa Cruz), and rabbit IgG (no. sc-2027; Santa Cruz). ChIP samples were prepared for sequencing using the Genomic DNA sample prep kit (Illumina) following manufacturers' protocols. The raw sequencing image data were analyzed by the Illumina analysis pipeline, aligned to the unmasked human reference genome (NCBI v36, hg18) using the ELAND software (Illumina) to generate sequence reads of 25-32 bps. These short reads were subsequently analyzed using HPeak. Statistically significant peaks, representing binding regions, were exported into wiggle files for visualization in the UCSC genome browser.

Calculating gene expression from RNA-Seq data. Transcriptome reads were trimmed to 32 nt by removing the first 2 bases and sufficient bases from the end necessary to yield a 32 mer. The 32-mer reads were aligned to the human genome plus 54-mer splice junctions generated by concatenating 28 bases from the end of the 5′ and 3′ splicing partner. This ensures that reads that map to the splice junction overlap the splice junction by 4 bases (Wang et al. Nature 456:470 [2008]; herein incorporated by reference in its entirety). The reads were aligned using Bowtie and allowing up to 2 bases of mismatch. Reads that did not yield a unique best hit, were discarded. Gene expression was calculated by first summing the coverage over all of the positions included in any isoform of the gene that is included in the UCSC mRNA dataset and then dividing by the number of positions included in the sum to yield the average coverage for the gene (Sultan et al. Science 321:956 [2008]; herein incorporated by reference in its entirety). Next, the average coverage was normalized by the number of reads mapping to the human genome in the sample and then multiplied by 1 million to yield a gene expression value in reads per kilobase million (RPKM).

Establishment of mate-pair filtering steps. The criteria described herein for filtering mate pairs encompassing a fusion boundary were selected for the following reasons. First, because the initial chimera candidates were derived from mappings against known transcripts, it is likely they have multiple alignments to the genome that do not correspond to an annotated transcript. Therefore, a mate pair was discarded if either of the mates failed to have a single unique best hit against the genome. If the mate pair does reveal single best hits, iteratetion through secondary mappings was done to ensure none of those reveal a mate pair combination that is in agreement with the expected insert size as this represents a more logical event. In addition to having a secondary hit residing approximately the insert size away on the same transcript, candidates were filtered within 50,000 kb on the genome, presuming this alignment does not overlap a different gene. For the remaining candidates, a filter was established that leverages the insert size between the mate pairs. It was expected that if multiple mate pairs were to support the same fusion event, their mappings will aggregate within the region flanking the fusion junction. An in silico insert size was calculated for each sample using mate pairs aligning to the same gene and the mean size of approximately 200 nt was found. Therefore, it was expected that if 2 mate pair were both encompassing the same breakpoint, the furthest apart that they could reside from one another would have to be nearly equivalent to the insert size. Next, it was observed that some candidates had identical mate pair reads that were in close proximity on the flow cell. These duplicates were likely an artifact of the analysis pipeline and resulted in the overrepresentation of a subset of chimeras. To circumvent this, for each chimera candidate, a nonredundant set of matepairs was generated supporting the predicted fusion event. Last, a requirement was set that a chimera have a minimum of 2 nonredundant mate pairs, unless there was supporting evidence of a mate pair spanning the fusion junction, to increase confidence in the nominated event.

Results. One of the most common classes of genetic alterations is gene fusions, resulting from chromosomal rearrangements (Futreal et al. Nat. Rev. 4:177 [2004]; herein incorporated by reference in its entirety). Approximately 80% of all known gene fusions are attributed to leukemias, lymphomas, and bone and soft tissue sarcomas that account for only 10% of all human cancers. In contrast, common epithelial cancers, which account for 80% of cancer-related deaths, can only be attributed to 10% of known recurrent gene fusions (Kumar-Sinha et al. Nat. Rev. 8:497 [2008]; Mitelman et al. Nat. Genet. 36:331 [2004]; Mitelman et al. Gene Chromosome Canc. 43:350 [2005]; each herein incorporated by reference in its entirety). However, the recent discovery of a recurrent gene fusion, TMPRSS2-ERG, in a majority of prostate cancers (Tomlins et al. Nature 448:595 [2007]; Tomlins et al. Science 310:644 [2005]; each herein incorporated by reference in its entirety), and EML4-ALK in nonsmall-cell lung cancer (NSCLC) (Soda et al. Nature 448:561 [2007]; herein incorporated by reference in its entirety), has expanded the realm of gene fusions as an oncogenic mechanism in common solid cancers. Also, the restricted expression of gene fusions to cancer cells makes them desirable therapeutic targets. One successful example is imatinib mesylate, or Gleevec, that targets BCR-ABL1 in chronic myeloid leukemia (CML) (Druker et al. New Engl. J. Med. 355:645 [2002]; Druker et al. Nat. Med. 2:561 [1996]; Kantarjian et al. New Engl. J. Med. 346:645 [2002]; each herein incorporated by reference in its entirety). Therefore, the identification of novel gene fusions in a broad range of cancers is of enormous therapeutic significance.

The lack of known gene fusions in epithelial cancers has been attributed to their clonal heterogeneity and to the technical limitations of cytogenetic analysis, spectral karyotyping, FISH, and microarray-based comparative genomic hybridization (aCGH). TMPRSS2-ERG was discovered by circumventing these limitations through bioinformatics analysis of gene expression data to nominate genes with marked overexpression, or outliers, a signature of a fusion event (Tomlins et al. Science 310:644 [2005]; herein incorporated by reference in its entirety). Building on this success, more recent strategies have adopted unbiased high-throughput approaches, with increased resolution, for genome-wide detection of chromosomal rearrangements in cancer involving BAC end sequencing (Volik et al. PNAS100:7696 [2003]; herein incorporated by reference in its entirety), fosmid paired-end sequences (Tuzun et al. Nat. Genet. 37:727 [2005]; herein incorporated by reference in its entirety), serial analysis of gene expression (SAGE)-like sequencing (Ruan et al. Genome Res. 17:828 [2007]; herein incorporated by reference in its entirety), and next-generation DNA sequencing (Campbell et al. Nat. Genet. 40:722 [2008]; herein incorporated by reference in its entirety). Despite unveiling many novel genomic rearrangements, solid tumors accumulate multiple nonspecific aberrations throughout tumor progression; thus, making causal and driver aberrations indistinguishable from secondary and insignificant mutations, respectively.

The deep unbiased view of a cancer cell enabled by massively parallel transcriptome sequencing has greatly facilitated gene fusion discovery. Integrating long and short read transcriptome sequencing technologies is an effective approach for enriching for “expressed” fusion transcripts (Maher et al. Nature 458:97 [2009]; herein incorporated by reference in its entirety). However, despite the success of this methodology, it required substantial overhead to leverage 2 sequencing platforms. Therefore, in this study, a single platform paired-end strategy was adapted to comprehensively elucidate novel chimeric events in cancer transcriptomes. Not only was using this single platform more economical, but it allowed a more comprehensively mapping of chimeric mRNA, to in on driver gene fusion products due to its quantitative nature, and to observe rare classes of transcripts that were overlapping, diverging, or converging.

Chimera Discovery via Paired-End Transcriptome Sequencing. Here, transcriptome sequencing was employed to restrict chimera nominations to “expressed sequences,” thus, enriching for potentially functional mutations. To evaluate massively parallel paired-end transcriptome sequencing to identify novel gene fusions, cDNA libraries were generated from the prostate cancer cell line VCaP, CML cell line K562, universal human reference total RNA (UHR; Stratagene), and human brain reference (HBR) total RNA (Ambion). Using the Illumina Genome Analyzer II, 16.9 million VCaP, 20.7 million K562, 25.5 million UHR, and 23.6 million HBR transcriptome mate pairs were generated (2×50 nt). The mate pairs were mapped against the transcriptome and categorized as (i) mapping to same gene, (ii) mapping to different genes (chimera candidates), (iii) nonmapping, (iv) mitochondrial, (v) quality control, or (vi) ribosomal (Table 10). Overall, the chimera candidates represent a minor fraction of the mate pairs, comprising of approximately <1% of the reads for each sample.

TABLE 10
Paired end summary statistics.

| | Lane 1 | Lane 2 | Lane 3 | Lane 4 | Total | Percentage |
|---|---|---|---|---|---|---|
| | VCaP | | | | | |
| Same gene | 3196295 | 3005894 | 2746073 | 2223151 | 11171423 | 65.5% |
| Fusion genes | 35249 | 31217 | 29465 | 22390 | 118311 | 0.7% |
| Ribosomal | 2509 | 2340 | 2243 | 1833 | 8925 | 0.1% |
| Non-mapping | 1445840 | 1333170 | 1261170 | 1143923 | 5184203 | 30.5% |
| Mitochondrial | 122035 | 114042 | 105123 | 84184 | 425384 | 2.5% |
| Quality Control (QC) | 22579 | 18351 | 14427 | 10675 | 66032 | [symbol] % |
| | 4824507 | 4505014 | 4158501 | 3486156 | 16974278 | |
| | K562 | | | | | |
| Same gene | 3774966 | 3756169 | 3737171 | 3505675 | 1477 [symbol] 071 | 71.3% |
| Fusion genes | 49665 | 49127 | 4782 [symbol] | 13390 | 16 [symbol] 908 | 0.9% |
| Ribosomal | 184435 | 182938 | 179565 | 167912 | 714850 | 3.4% |
| Non-mapping | 1031211 | 1047680 | 1080454 | 1073374 | 423 [symbol] 729 | 20.4% |
| Mitochondrial | 208455 | 209451 | 208877 | 195094 | 822877 | 4.0% |
| Quality Control (QC) | 26 | 19 | 38 | 37 | 114 | 0.0% |
| | 5248758 | 5245384 | 525393 [symbol] | 4955482 | 20734549 | |

| | Lane 1 | Lane 2 | Lane 3 | Total | Percentage |
|---|---|---|---|---|---|
| | UHR | | | | |
| Same gene | 8176075 | 6083374 | 6924187 | 18182636 | 71.2% |
| Fusion genes | 53671 | 52328 | 51285 | 157204 | 0.5% |
| Ribosomal | 231218 | 228336 | 221872 | 681425 | 2.7% |
| Non-mapping | 17 [symbol] 29 [symbol] | 1569245 | 1619 [symbol] 57 | 5111292 | 20.5% |
| Mitochondrial | 472404 | 463054 | 453238 | 1388706 | 5.4% |
| Quality Control (QC) | 2645 | 5442 | 2917 | 14006 | 0.1% |
| | | | Total | 25535269 | |
| | Brain | | | | |
| Same gene | 5462592 | 5173159 | 492 [symbol] 236 | 15 [symbol] 39 | 65.8% |
| Fusion genes | 48116 | 37624 | 36344 | 114084 | 0.5% |
| Ribosomal | 157576 | 149854 | 144159 | 451719 | 1.9% |
| Non-mapping | 1776145 | 1578741 | 18 [symbol] 4155 | 5339341 | 22.6% |
| Mitochondrial | 758158 | 715153 | 677967 | 2152310 | 9.1% |
| Quality Control (QC) | 6259 | 4570 | 5336 | 18165 | 0.1% |
| | | | Total | 23529858 | |

[symbol]

indicates data missing or illegible when filed

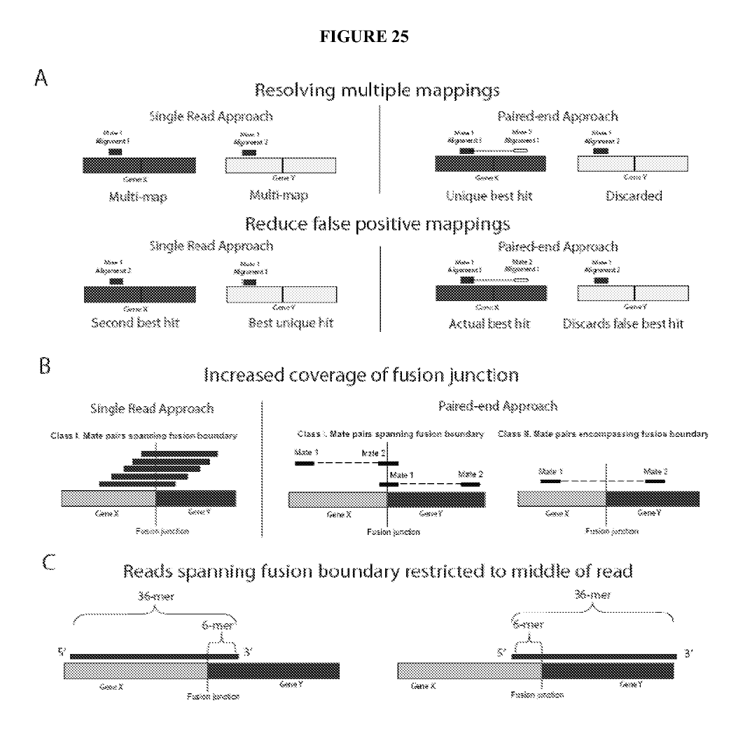

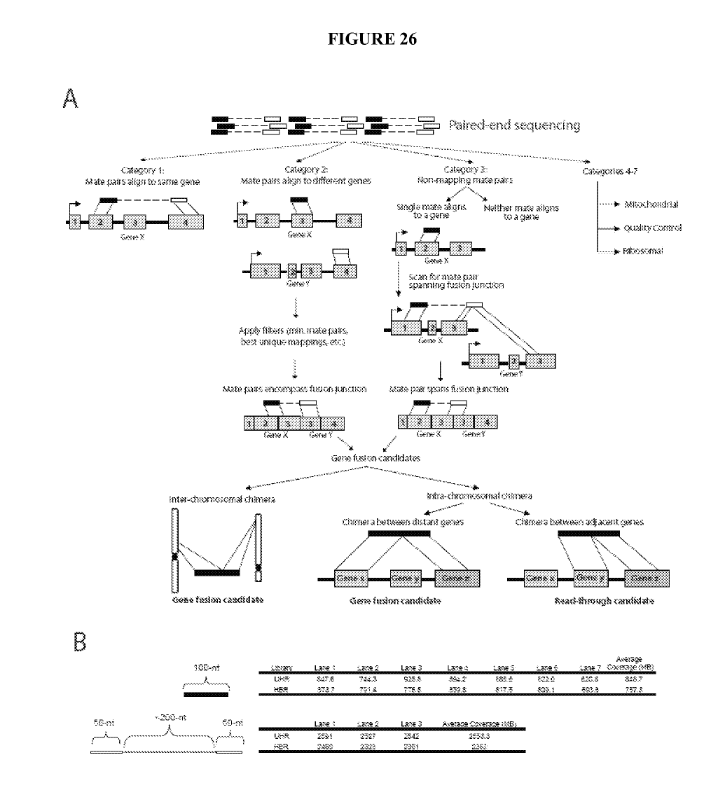

A paired-end strategy was believed to offer multiple advantages over single read based approaches such as alleviating the reliance on sequencing the reads traversing the fusion junction, increased coverage provided by sequencing reads from the ends of a transcribed fragment, and the ability to resolve ambiguous mappings (FIG. 25). Therefore, to nominate chimeras, each of these aspects was leveraged in the bioinformatics analysis. Focus was kept on both mate pairs encompassing and/or spanning the fusion junction by analyzing 2 main categories of sequence reads: chimera candidates and nonmapping (FIG. 26). The resulting chimera candidates from the nonmapping category that span the fusion boundary were merged with the chimeras found to encompass the fusion boundary revealing 119, 144, 205, and 294 chimeras in VCaP, K562, HBR, and UHR, respectively.

Comparison of a Paired-End Strategy Against Existing Single Read Approaches. To assess the merit of adopting a paired-end transcriptome approach, results were compared against existing single read approaches. Although current RNA sequencing (Seq) studies have been using 36-nt single reads (Marioni et al. Genome Res. 18:1509 [2008]; Mortazavi et al. Nat. Methods 5:621 [2008]; each herein incorporated by reference in its entirety), the likelihood of spanning a fusion junction was increased by generating 100-nt long single reads using the Illumina Genome Analyzer II. Also, this length was chosen because it would facilitate a more comparable amount of sequencing time as required for sequencing both 50-nt mate pairs. In total, 7.0, 59.4, and 53.0 million 100-nt transcriptome reads were generated for VCaP, UHR, and HBR, respectively, for comparison against paired-end transcriptome reads from matched samples.

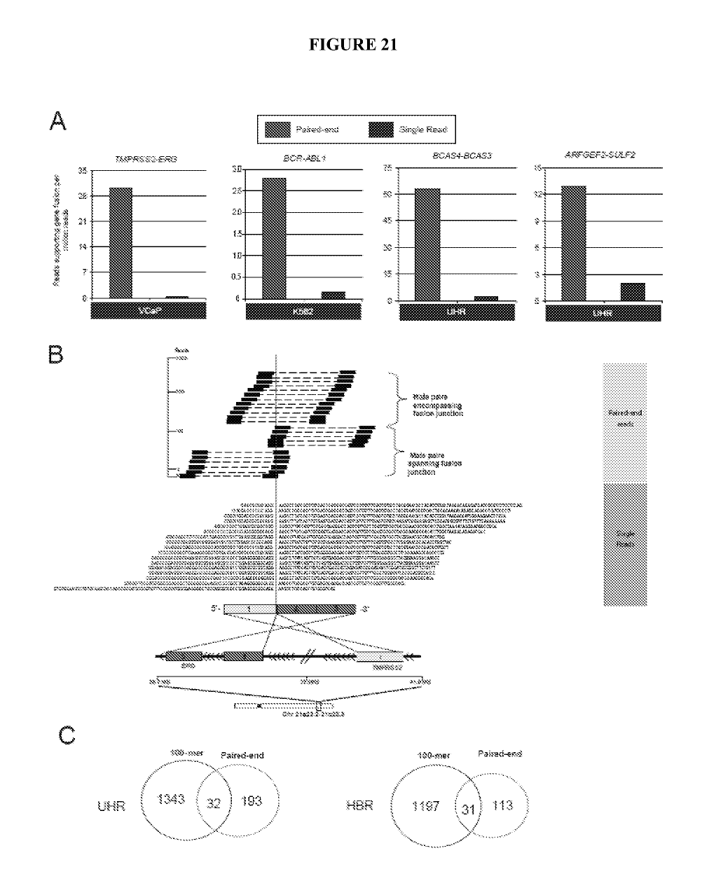

Because the UHR is a mixture of cancer cell lines, there was an expectation to find numerous previously identified gene fusions. Therefore, the depth of coverage of a paired-end approach against long single reads was first assessed by directly comparing the normalized frequency of sequence reads supporting 4 previously identified gene fusions (TMPRSS2-ERG (Tomlins et al. Nature 448:595 [2007]; Tomlins et al. Science 310:644 [2005]; each herein incorporated by reference in its entirety), BCR-ABL1 (Shtivelman et al. Nature 315:550 [1985]; herein incorporated by reference in its entirety), BCAS4-BCAS3 (Barlund et al. Gene Chromosome Canc. 35:311 [2002]; herein incorporated by reference in its entirety), and ARFGEF2-SULF2 (Hampton et al. Genome Res. 19:167 [2009]; herein incorporated by reference in its entirety)). As shown in FIG. 21A, a marked enrichment of paired-end reads was observed as compared with long single reads for each of these well characterized gene fusions.

TMPRSS2-ERG was observed to have a >10-fold enrichment between paired-end and single read approaches. The schematic representation in FIG. 21B indicates the distribution of reads confirming the TMPRSS2-ERG gene fusion from a single flow cell lane of both paired-end and single read sequencing. The longer reads improve the number of reads spanning known gene fusions. For example, had a single 36-mer been sequenced, 11 of the 17 chimeras, shown in the bottom portion of the long single reads, would not have spanned the gene fusion boundary, but instead, would have terminated before the junction and, therefore, only aligned to TMPRSS2. However, despite the improved results from longer single reads, this generated only 17 chimeric reads from 7.0 million sequences. In contrast, paired-end sequencing resulted in 552 reads supporting the TMPRSS2-ERG gene fusion from approximately 17 million sequences.

Because sequence based evidence was used to nominate a chimera, it was hypothesized that the approach providing the maximum nucleotide coverage is more likely to capture a fusion junction. An in silico insert size was calculated for each sample using mate pairs aligning to the same gene, and it was found that the mean insert size was approximately 200 nt. Then, the total coverage from single reads (coverage is equivalent to the total number of pass filter reads against the read length) was compared with the paired-end approach (coverage is equivalent to the sum of the insert size with the length of each read) (FIG. 26B). Overall, an average coverage of 848.7 and 757.3 MB was observed, using single read technology, compared with 2,553.3 and 2,363 MB from paired-end in UHR and HBR, respectively. This increase in approximately 3-fold coverage in the paired-end samples compared with the long read approach, per lane, could explain the increased dynamic range observed using a paired-end strategy.

Next it was desired to identify chimeras common to both strategies. The long read approach nominated 1,375 and 1,228 chimeras, whereas with a paired-end strategy, only 225 and 144 chimeras in UHR and HBR were nominated, respectively. As shown in the Venn diagram (FIG. 21C), there were 32 and 31 candidates common to both technologies for UHR and HBR, respectively. Within the common UHR chimeric candidates, previously identified gene fusions BCAS4-BCAS3, BCR-ABL1, ARFGEF2-SULF2, and RPS6 KB1-TMEM49 (Ruan et al. Genome Res. 17:828 [2007]; herein incorporated by reference in its entirety) were observed. The remaining chimeras, nominated by both approaches, represent a high fidelity set. Therefore, to further assess whether a paired-end strategy has an increased dynamic range, the ratio of normalized mate pair reads was compared against single reads for the remaining chimeras common to both technologies. It was observed that 93.5 and 93.9% of UHR and HBR candidates, respectively, had a higher ratio of normalized mate pair reads to single reads (Table 11), confirming the increased dynamic range offered by a paired-end strategy. It was hypothesized that the greater number of nominated candidates specific to the long read approach represents an enrichment of false positives, as observed when using the 454 long read technology (Maher et al. Nature 458; 97 [2009]; Zhao et al. PNAS 106:1886 [2009]; each herein incorporated by reference in its entirety).

TABLE 11
Chimera candidates nominated by 100-nt reads and paired-end sequencing.

| | | | | | Paired End | Long Read | |
|---|---|---|---|---|---|---|---|
| Sample | 5p Gene | 5p [symbol] | [symbol] Gene | 3p [symbol] | [symbol] | [symbol] | [symbol] |
| HBR | [symbol] E [symbol] | NM_0 [symbol] 5 [symbol] 78 | LOC34923 [symbol] | NM_182635 | 0.26 | 0.0169 | 15.39 |
| | DPH1 | NM_001383 | OVCA2 | NM_080622 | 0.22 | 0.0169 | 1 [symbol] . [symbol] 2 |
| | H [symbol] | NM_213 [symbol] | WNK1 | NM_013979 | 0.17 | 0.0169 | 10.06 |
| | PRH1 | NM_ [symbol] 62 [symbol] | PRR4 | NM_007244 | 0.17 | 0.0169 | 10.06 |
| | [symbol] | NM_024326 | P [symbol] D | NM_002779 | 0.13 | 0.0169 | 7.7 |
| | M [symbol] 2474 | NM_023931 | FLJ23435 | NM_ [symbol] 4671 | 0.22 | 0.0337 | 6.53 |
| | INPP [symbol] A | NM_005539 | NKX5-2 | NM_177400 | 0.43 | 0.0674 | 5.38 |
| | [symbol] | NM_018455 | C16orf61 | NM_0201 [symbol] | 0.09 | 0.0169 | 5.33 |
| | [symbol] NF1LK2 | NM_015191 | PPP2R1B | NM_181699 | 0.09 | 0.0169 | 5.33 |
| | GFAP | NM_ [symbol] | TP63INP2 | NM_021202 | 0.09 | 0.0169 | 5.33 |
| | HLA-E | NM_ [symbol] 5516 | HLA-C | NM_002117 | 0.09 | 0.0169 | 5.33 |
| | A [symbol] C1 | NM_152265 | C9orf37 | NM_032937 | 0.09 | 0.0169 | 5.33 |
| | T [symbol] | NM_ [symbol] 20 [symbol] | ZNF269 | NM_0 [symbol] 5741 | 0.09 | 0.0169 | 5.33 |
| | COG1 | NM_018714 | FAM1 [symbol] | NM_032837 | 0.09 | 0.0169 | 5.33 |
| | [symbol] | NM_024102 | OVGP1 | NM_002557 | 0.09 | 0.0169 | 5.33 |
| | APE [symbol] | NM_0 [symbol] 1640 | RNF123 | NM_022 [symbol] 64 | 0.09 | 0.0169 | 5.33 |
| | [symbol] 12 | NM_ [symbol] 2989 | HDAC1 [symbol] | NM_0 [symbol] 2 [symbol] 1 [symbol] | 0.09 | 0.0169 | 5.33 |
| | [symbol] | NM_0 [symbol] 3910 | PTCD1 | NM_015546 | 0.13 | 0.0337 | 3.86 |
| | PHPT [symbol] | NM_014172 | EDF1 | NM_1532 [symbol] | 0.13 | 0.0337 | 3. [symbol] 5 |
| | C [symbol] | NM_182516 | AP [symbol] | NM_0 [symbol] 29 | 0.22 | 0.0674 | 3.27 |
| | [symbol] | NM_1 [symbol] 9295 | [symbol] | NM_0013 [symbol] 2 | 0.09 | 0.0337 | 2. [symbol] |
| | [symbol] | NM_015407 | ACY1 | NM_0 [symbol] 6 | 0.09 | 0.0337 | 2. [symbol] 8 |
| | [symbol] 27 | NM_518971 | EF4E3 | NM_173359 | 0.09 | 0.0337 | 2. [symbol] 3 |
| | EF4E3 | NM_173369 | GPR27 | NM_013971 | 0.13 | 0.0505 | 2.58 |
| | ABC [symbol] | NM_0 [symbol] 71 [symbol] 5 | ACCN [symbol] | NM_004759 | 0.17 | 0.0674 | 2.33 |
| | EF2A [symbol] | NM_014413 | JTV1 | NM_ [symbol] 303 | 0.17 | 0.0674 | 2.53 |
| | TUB [symbol] | NM_0 [symbol] 085 | TUB [symbol] | NM_ [symbol] 87 | 0.09 | 0.0505 | 1.79 |
| | NKX5-2 | NM_1774 [symbol] | [symbol] A | NM_0 [symbol] 538 | 0.09 | 0.0505 | 1.79 |
| | BPTF | NM_0 [symbol] 4459 | [symbol] PNA2 | NM_002266 | 0.22 | 0.1347 | 1.64 |
| | CENPT | NM_02 [symbol] 082 | NUTF2 | NM_005796 | 0.09 | 0.1179 | 0.77 |
| | CHAD | NM_ [symbol] 1257 | FLJ20925 | NM_025149 | 0.09 | 0.2594 | 0.34 |
| UHR | BCA34 | NM_017543 | [symbol] CA [symbol] | NM_001099 [symbol] 32 | 46.2106 | 2.8297 | 15.34 |
| | [symbol] CR | NM_021674 | ABL [symbol] | NM_ [symbol] 7313 | 2.7414 | 0.1887 | 14.53 |
| | ANP325 | NM_005401 | DALR | NM_0 [symbol] 4 [symbol] | 2.3497 | 0.1887 | 12.46 |
| | RP [symbol] | NM_ [symbol] 3141 | TMEM49 | NM_ [symbol] 093 [symbol] | 1.9581 | 0.1887 | 10.36 |
| | [symbol] D1 | NM_194249 | WDR [symbol] | NM_017706 | 1.6655 | 0.1887 | 5.31 |
| | FAD [symbol] | NM_ [symbol] 134 [symbol] 2 | FAD [symbol] 2 | NM_ [symbol] 4255 | 1.5565 | 0.1887 | 5.31 |
| | NUP214 | NM_ [symbol] | [symbol] | NM_175678 | 2.3497 | 0.3773 | 6.23 |
| | ADCK4 | NM_024876 | NUMB [symbol] | NM_ [symbol] 4756 | 2.3497 | 0.3773 | 6.23 |
| | [symbol] 1 | NM_162289 | ZNF562 | NM_017656 | 1.1749 | 0.1887 | 6.23 |
| | VAMP [symbol] | NM_0 [symbol] 3761 | VAMP [symbol] | NM_ [symbol] 634 | 1.1749 | 0.1887 | 6.23 |
| | TUBA [symbol] | NM_ [symbol] 532704 | K-ALPHA-1 | NM_006 [symbol] 82 | 1.1749 | 0.01887 | 6.23 |
| | GA [symbol] [symbol] | NM_0 [symbol] 20 | RASA3 | NM_067 [symbol] 68 | 13.7856 | 2.4524 | 5. [symbol] 9 |
| | FLJ14540 | NM_ [symbol] 2815 | PEPD | NM_000255 | 2.3497 | 0.56 [symbol] | 4.15 |
| | ZFP41 | NM_173832 | GL [symbol] 4 | NM_1 [symbol] 8455 | 1.5565 | 0.3773 | 4.1 [symbol] |
| | DNAJB7 | NM 145174 | LOC63929 | NM 022995 | 0.7833 | 0.1567 | 4.1 [symbol] |
| | MCFD2 | NM 1 [symbol] 9279 | TTC7A | NM 020458 | 0.7833 | 0.1567 | 4.1 [symbol] |
| | HEXB | NM_000521 | GFM2 | NM_170591 | 0.7833 | 0.1567 | 4.1 [symbol] |
| | DGCR [symbol] | NM_02272 [symbol] | HTF9 [symbol] | NM_182984 | 0.7833 | 0.1567 | 4.1 [symbol] |
| | C11orf2 | NM_01326 [symbol] | TM7 [symbol] F2 | NM_003273 | 0.7833 | 0.1567 | 4.1 [symbol] |
| | AP4B1 | NM_00 [symbol] 94 | R [symbol] N1 | NM_018364 | 0.7833 | 0.1567 | 4.1 [symbol] |
| | PGLA2 | NM 0 [symbol] | CDC42EP2 | NM 0 [symbol] 6779 | 0.7833 | 0.1567 | 4.1 [symbol] |
| | TM [symbol] X | NM 021109 | TM [symbol] | NM 183549 | 1. [symbol] 581 | [symbol] .565 | 3.46 |
| | ARFGEF2 | NM 00642 [symbol] | [symbol] ULF2 | NM 198595 | 8.6155 | 2.5413 | 3.27 |
| | PLCXD2 | NM 153269 | PHLD62 | NM 1457 [symbol] | 1.3748 | 0.3773 | 3.12 |
| | D [symbol] C2A | NM 003586 | FLJ90652 | NM 17361 [symbol] | 0.7833 | 0.3773 | 2.08 |
| | CGI-96 | NM 015703 | SERHL | NM 170594 | 0.7833 | 0.3773 | 2.08 |
| | CRIP2 | NM 001312 | CRIP1 | NM 001 [symbol] 11 | 0.7833 | 0. [symbol] 65 | 1.38 |
| | HLA-G | NM 002127 | HLA- [symbol] | NM 002117 | 0.7833 | 0.7545 | 1.04 |
| | ZNF276 | NM 004924 | C16orf7 | NM 004913 | 0.7833 | 0.7545 | 1.04 |
| | ACTN4 | NM 004913 | ACTN1 | NM 001102 | 0.7833 | 0.7545 | 1.04 |
| | C16orf [symbol] | NM 002115 | ZNF27 [symbol] | NM 152287 | 0.7833 | 0.7545 | 1.04 |
| | HLA- [symbol] | NM 0 [symbol] 29 | HLA-B | NM [symbol] 14 | 0.7833 | 0.9433 | 0. [symbol] 4 |
| | FLJ14346 | NM 0 [symbol] 29 | N [symbol] MA [symbol] E3 | NM 017751 | 0.7833 | 1.5092 | [symbol] |
| | [symbol] -ALPHA- | NM-0 [symbol] 082 | TUBA3 | NM_006009 | 0.7833 | 2.2538 | 0.35 |
| | [symbol] | | | | | | |

[symbol]

indicates data missing or illegible when filed

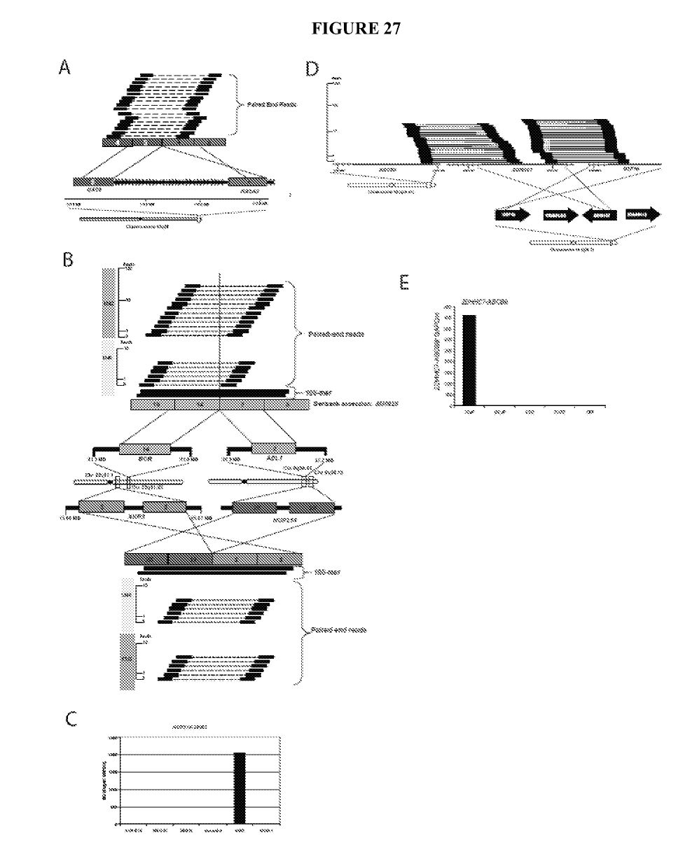

Paired-End Approach Reveals Novel Gene Fusions. Among the top chimeras nominated from VCaP, HBR, UHR, and K562, many were already known, including TMPRSS2-ERG, BCAS4-BCAS3, BCR-ABL1, USP10-ZDHHC7, and ARFGEF2-SULF2. Also ranking among these well known gene fusions in UHR was a fusion on chromosome 13 between GAS6 and RASA3 (FIG. 27A and Table 11). The fact that GAS6-RASA3 ranked higher than BCR-ABL1 indicates that it may be a driving fusion in one of the cancer cell lines in the RNA pool.

Another observation was that there were 2 candidates among the top 10 found in both UHR and K562. Hematological malignancies are not considered to have multiple gene fusion events. In addition to BCR-ABL1, it was possible to detect a previously undescribed interchromosomal gene fusion between exon 23 of NUP214 located at chromosome 9q34.13 with exon 2 of XKR3 located on chromosome 22. Both of these genes reside on chromosome 22 and 9, in close proximity, to BCR and ABU, respectively (FIG. 27B). The presence of NUP214 XKR3 in K562 cells was confirmed using qRT-PCR, but it was not possible to detect it across an additional 5 CML cell lines tested (SUP-B15, MEG-01, KU812, GDM-1, and Kasumi-4) (FIG. 27C). This indicates that NUP214-XKR3 is a “private” fusion that originated from additional complex rearrangements after the translocation that generated BCR-ABL1 and a focal amplification of both gene regions.

Although it was possible to detect BCR-ABL1 and NUP214-XKR3 in both UHR and K562, there was a marked reduction in the mate pairs supporting these fusions in UHR. Although a diluted signal is expected, because UHR is pooled samples, it provides evidence that pooling samples can serve as a useful approach for nominating top expressing chimeras, and potentially enrich for “driver” chimeras.

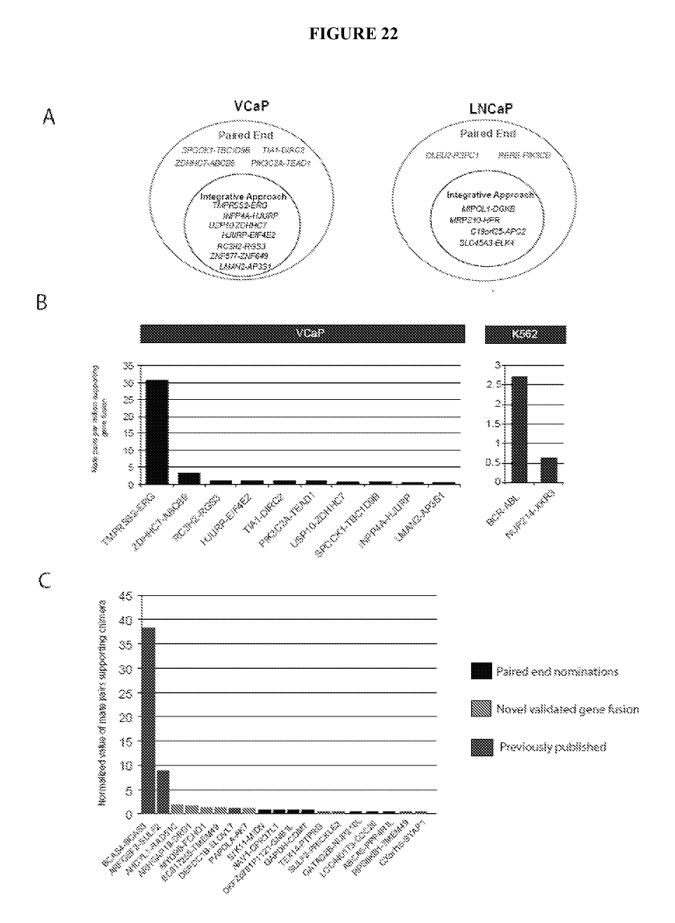

Previously Undescribed Prostate Gene Fusions. Previous work using integrative transcriptome sequencing to detect gene fusions in cancer revealed multiple gene fusions, demonstrating the complexity of the prostate transcriptomes of VCaP and LNCaP (Maher et al. Nature 458:97 [2009]; herein incorporated by reference in its entirety). Here, the comprehensiveness of a paired-end strategy on the same cell lines was exploited to reveal novel chimeras. In the circular plot shown in FIG. 22A, all experimentally validated paired-end chimeras are displayed in the larger circle. All of the previously discovered chimeras in VCaP and LNCaP comprised a subset of the paired-end candidates, as displayed in the inner circle.

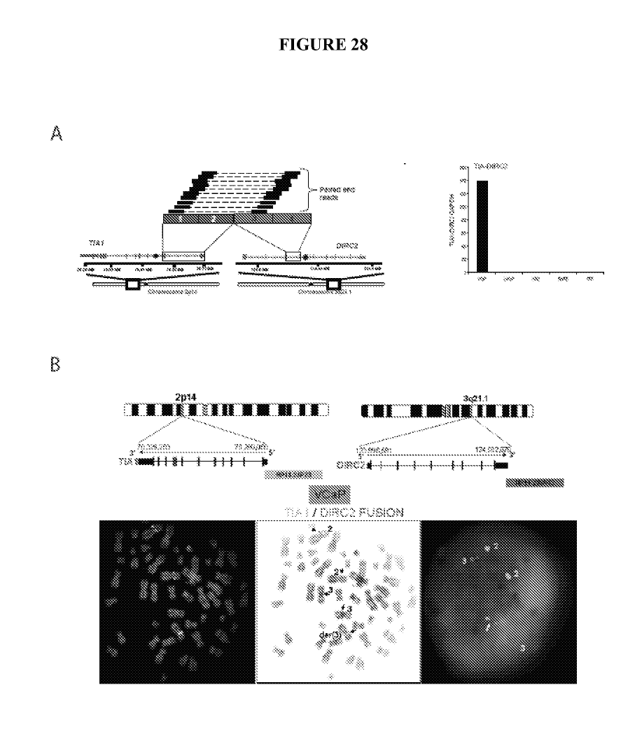

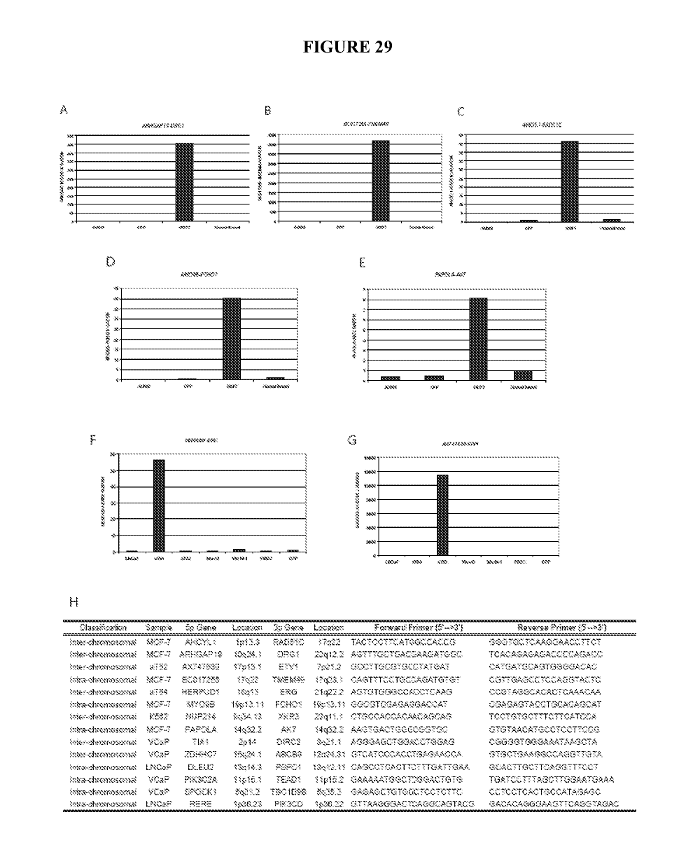

TMPRSS2-ERG was the top VCaP candidate. In addition to “rediscovering” the USP10-ZDHHC7, HJURP-INPP4A, and EIF4E2-HJURP gene fusions, a paired-end approach revealed several previously undescribed gene fusions in VCaP. One such example was an interchromosomal gene fusion between ZDHHC7, on chromosome 16, with ABCB9, residing on chromosome 12, that was validated by qRT-PCR (FIG. 27D). The 5′ partner, ZDHHC7, had previously been validated as a complex intrachromosomal gene fusion with USP10 (Maher et al. Nature 458:97 [2009]; herein incorporated by reference in its entirety). Both fusions have mate pairs aligning to the same exon of ZDHHC7 (Maher et al. Nature 458:97 [2009]; herein incorporated by reference in its entirety), indicating that their breakpoints are in adjacent introns (FIG. 27D). Another previously undescribed VCaP interchromosomal gene fusion was between exon 2 of TIA1, residing on chromosome 2, with exon 3 of DIRC2, or disrupted in renal carcinoma 2, located on chromosome 3. TIA1-DIRC2 was validated by qRT-PCR and FISH (FIG. 28). In total, an additional 4 VCaP and 2 LNCaP chimeras were confirmed (FIG. 29). Overall, these fusions demonstrate that paired-end transcriptome sequencing can nominate candidates that have eluded previous techniques, including other massively parallel transcriptome sequencing approaches.

Distinguishing Causal Gene Fusions from Secondary Mutations. The next objective was to determine whether the dynamic range provided by paired-end sequencing can distinguish known high level “driving” gene fusions, such as known recurrent gene fusions BCR-ABL1 and TMPRSS2-ERG, from lower level “passenger” fusions. To evaluate this, the normalized mate pair coverage was plotted at the fusion boundary for all experimentally validated gene fusions for the 2 cell lines that were sequenced harboring recurrent gene fusions, VCaP and K562. As shown in FIG. 22B, both driver fusions, TMPRSS2-ERG and BCR-ABL1, were observed to show the highest expression among the validated chimeras in VCaP and K562, respectively. This demonstrates a paired-end nomination strategy for selecting putative driver gene fusions among private nonspecific private gene fusions, because many of these were experimentally tested and shown to lack detectable levels of expression across a panel of samples (Maher et al. Nature 458:97 [2009]; herein incorporated by reference in its entirety).

Previously Undescribed Breast Cancer Gene Fusions. The ability to detect previously undescribed prostate gene fusions in VCaP and LNCaP demonstrated the comprehensiveness of paired-end transcriptome sequencing compared with an integrated approach, using short and long transcriptome reads. Therefore a paired-end approach was applied to detect novel breast cancer gene fusions. To accomplish this, paired-end transcriptome sequencing of the breast cancer cell line MCF-7 was conducted. MCF-7 has been mined for fusions using numerous approaches such as expressed sequence tags (ESTs) (Hahn et al. PNAS101:13257 [2004]; herein incorporated by reference in its entirety), array CGH (Shadeo et al. Breast Cancer Res. 8:R9 [2006]; herein incorporated by reference in its entirety), single nucleotide polymorphism arrays (Huang et al. Hum. Genom. 1:287 [2004]; herein incorporated by reference in its entirety), gene expression arrays (Neve et al. Cancer Cell 10:515 [2006]; herein incorporated by reference in its entirety), end sequence profiling (Hampton et al. Genome Res. 19:167 [2009]; Volik et al. Genome Res. 16:394 [2006]; each herein incorporated by reference in its entirety), and paired-end diTag (PET) (Ruan et al. Genome Res. 17:828 [2007]; herein incorporated by reference in its entirety).

A histogram (FIG. 22C) of the top ranking MCF-7 candidates highlights BCAS4-BCAS3 and ARFGEF-SULF2 as the top 2 ranking candidates, whereas other previously reported candidates, such as SULF2-PRICKLE, DEPDC1B-ELOVL7, RPS6 KB1-TMEM49, and CXorf15-SYAP1, were interspersed among a comprehensive list of previously undescribed putative chimeras. To confirm that these previously undescribed nominations were not false positives, 2 interchromosomal and 3 intrachromosomal candidates were experimentally validated using qRT-PCR (FIG. 29). Overall, not only was a paired-end approach able to detect gene fusions that have eluded numerous existing technologies, it revealed 5 previously undescribed mutations in breast cancer.

RNA-Based Chimeras. Although many of the inter and intrachromosomal rearrangements that were nominated were found within a single sample many chimeric events were observed to be shared across samples. 13 chimeric events were identified as common to UHR, VCaP, K562, and HBR (Table 12). Via heatmap representation (FIG. 3A) of the normalized frequency of mate pairs supporting each chimeric event, these events are observed to be broadly transcribed, in contrast to the top 13 restricted chimeric events. Also, 100% of the broadly expressed chimeras resided adjacent to one another on the genome, whereas only 7.7% of the restricted candidates were neighboring genes. This discrepancy can be explained by the enrichment of inter and intrachromosomal rearrangements in the restricted set.

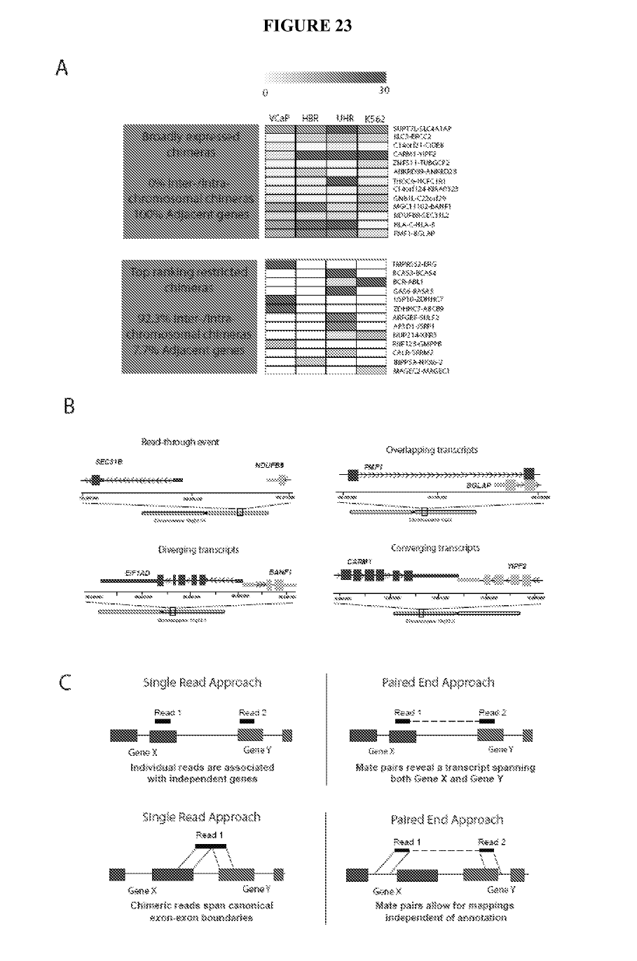

Unlike previously characterized restricted read-throughs, such as SLC45A3-ELK4 (Maher C A, et al. (2009) Nature 458:97-101), which are found adjacent to one another, but in the same orientation, the majority of the broadly expressed chimera candidates resided adjacent to one another in different orientations. Therefore, these events were catagorized as (i) read-throughs, adjacent genes in the same orientation, (ii) diverging genes, adjacent genes in opposite orientation whose 5′ sites are in close proximity, (iii) convergent genes, adjacent genes in opposite orientation whose 3′ ends are in close proximity, and (iv) overlapping genes, adjacent genes who share common exons (FIG. 3B). Based on this classification, 1 read-through, 2 convergent genes, 6 divergent genes, and 4 overlapping genes were found. Also, approximately 84.6% of these chimeras had at least 1 supporting EST, providing independent confirmation of the event (Table 12). In contrast to paired-end, single read approaches would likely miss these instances as each mate would have aligned to their respective genes based on the current annotations (FIG. 23C). Also, these instances may represent extensions of a transcriptional unit, which would not be detectable by a single read approach that identifies chimeric reads that span exon boundaries of independent genes. Overall, many of these broadly expressed RNA chimeras represent instances where mate pairs are revealing previously undescribed annotation for a transcriptional unit.

TABLE 12
Chimeras nominated in all samples (VCaP, K562, and Brain).

| 5p Gene | 5p Refseq | 3p Gene | 3p Refseq | Category | EST confirmation |
|---|---|---|---|---|---|
| CARM1 | NM_199141 | YIPF2 | NM_024029 | Converging | Yes |
| MGC11102 | NM_032325 | BANF1 | NM_0 [symbol] 550 | Diverging | Yes |
| SLC4A1AP | NM_018158 | SUPT7L | NM_0149 [symbol] | Diverging | Yes |
| ERCC2 | NM_030400 | KLC3 | NM_177417 | Converging | Yes |
| PMF1 | NM_037221 | BGLAP | NM_199173 | Overlapping | Yes |
| THCD6 | NM_024339 | HCFC [symbol] 1 | NM_0178 [symbol] | Diverging | Yes |
| NDLF55 | NM_035224 | SEC31L2 | NM_015490 | Read-through | Yes |
| ANKRD [symbol] | NM_016455 | ANKRD23 | NM_144994 | Diverging | No |
| C14orf124 | NM_020195 | KIAA [symbol] 323 | NM_015299 | Overlapping | Yes |
| C14orf21 | NM_174913 | [symbol] IDES | NM_014430 | Diverging | No |
| ZNF511 | NM_145906 | TUBGCP2 | NM_026659 | Diverging | Yes |

[symbol]

indicates data missing or illegible when filed

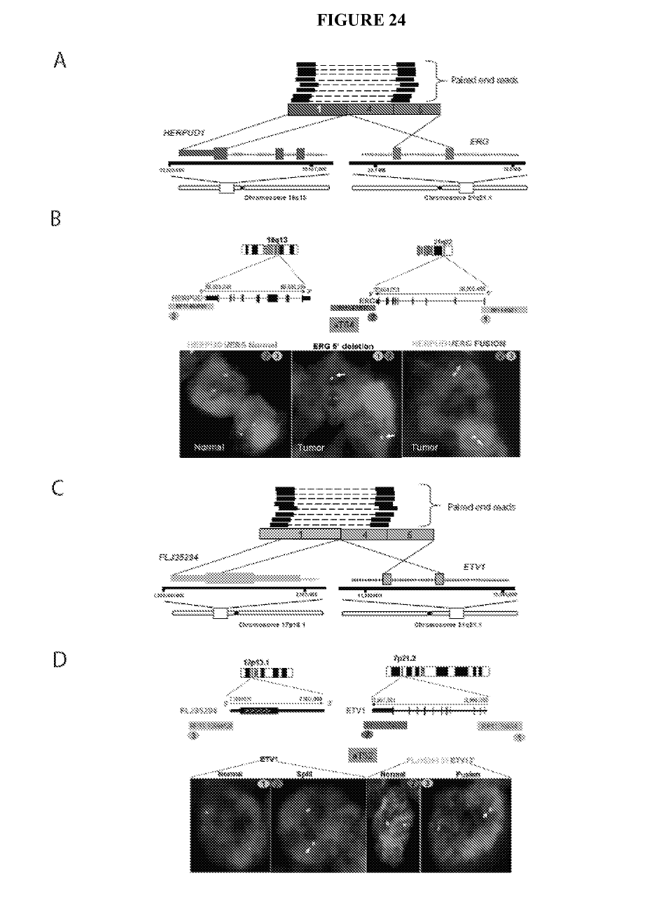

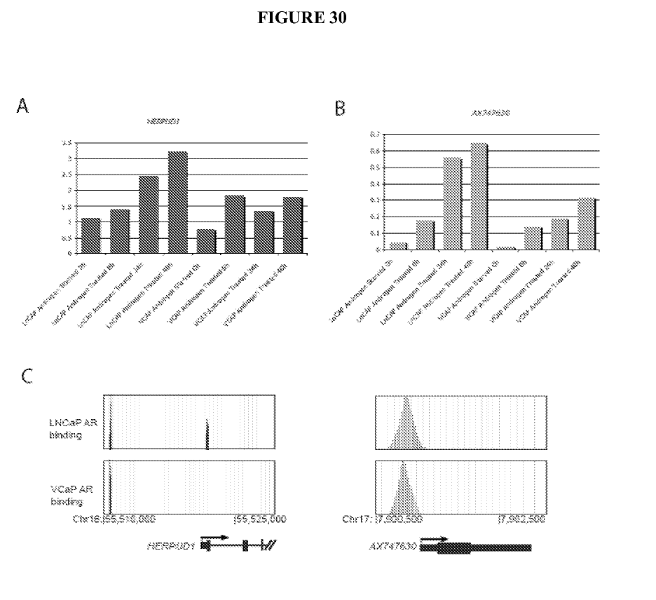

Previously Undescribed ETS Gene Fusions in Clinically Localized Prostate Cancer. Given the high prevalence of gene fusions involving ETS oncogenic transcription factor family members in prostate tumors, paired-end transcriptome sequencing was applied for gene fusion discovery in prostate tumors lacking previously reported ETS fusions. For 2 prostate tumors, aT52 and aT64, 6.2 and 7.4 million transcriptome mate pairs were generated, respectively. In aT64, HERPUD1, residing on chromosome 16, juxtaposed in front of exon 4 of ERG (FIG. 24A), which was validated by qRT-PCR (FIG. 29) and FISH (FIG. 24B). This represents the third 5′ fusion partner for ERG, after TMPRSS2 (Tomlins et al. Science 310:644 [2005]; herein incorporated by reference in its entirety) and SLC45A3 (Han et al. Cancer Res. 68:7629 [2008]; herein incorporated by reference in its entirety), and presumably, HERPUD1 also mediates the overexpression of ERG in a subset of prostate cancer patients. Also, just as TMPRSS2 and SLC45A3 have been shown to be androgen regulated by qRT-PCR (Tomlins et al. Nature 448:595 [2007]; herein incorporated by reference in its entirety), HERPUD1 expression, via RNASeq, to be responsive to androgen treatment (FIG. 30). Also, ChIP-Seq analysis revealed androgen binding at the 5′ end of HERPUD1 (FIG. 30).

Also, in the second prostate tumor sample (aT52), an interchromosomal gene fusion was discovered between the 5′ end of a prostate cDNA clone, AX747630, residing on chromosome 17, with exon 4 of ETV1, located on chromosome 7 (FIG. 246), which was validated via qRT-PCR (FIG. 29) and FISH (FIG. 24D). This fusion has previously been reported in an independent sample found by a fluorescence in situ hybridization screen (Han et al. Cancer Res. 68:7629 [2008]; herein incorporated by reference in its entirety); thus, demonstrating that it is recurrent in a subset of prostate cancer patients. As previously reported, gene expression via RNA-Seq confirmed that AX747630 is an androgen-inducible gene (FIG. 30). Also, ChIP-Seq revealed androgen occupancy at the 5′ end of AX747630 (FIG. 30).

Effectiveness of paired-end filtering steps. The chimera candidates, comprised of mate pairs that align to different genes, were subjected to a series of filters incorporating insert size, duplicate reads, and ambiguous mappings to reduce potential false positives. To confirm the effectiveness of the filters, 12 candidates were tested that did not pass the filters, and all failed qRT-PCR validation. This confirms that these filters are removing false positive nominations.

Paracentric inversion generates novel universal human reference (UHR) gene fusion, GAS6-RASA3. The gene fusion between GAS6 and RASA3 residing on chromosome 13 was of particular interest. The fact that GAS6-RASA3 ranked higher than BCR-ABL1 indicates that it is a driving fusion in one of the cancer cell lines in the RNA pool. GAS6 is a gamma-carboxyglutamic acid (Gla)-containing protein believed to stimulate cell proliferation. It resides approximately 200 MB, in opposite orientation and separated by FAM70B, from RASA3 indicating that this fusion gene is generated by a small paracentric inversion. RASA3 is a member of the GAP1 family of GTPase-activating proteins. Overall, GAS6-RASA3 is one of many novel gene fusions that sheds light into the tumorigenesis of one of the anonymous cancer cell lines within the UHR pool.

Novel interchromosomal VCaP gene fusions, TIA1-DIRC2. One novel VCaP interchromosomal gene fusion found by a paired-end strategy was between exon 2 of TIA1, residing on chromosome 2, with exon 3 of DIRC2, or disrupted in renal carcinoma 2, located on chromosome 3. TIA1-DIRC2 was validated by qRTPCR and FISH (FIG. 28). The splicing regulator, TIA1, is a member of a RNA-binding protein family that has nucleolytic activity against cytotoxic lymphocyte (CTL) target cells and could have a role in inducing apoptosis. The present invention is not limited to a particular mechanism. Indeed, an understanding of the mechanism is not necessary to practice the present invention. Nonetheless, the disruption of DIRC2 has been associated with haplo-insufficiency, which could provide mechanism for tumor growth in renal cell carcinoma (Bodmer et al. Hum. Mol. Genet. 11:641 [2002]; herein incorporated by reference in its entirety).

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

We claim:

1. A method for identifying prostate cancer in a patient comprising:
(a) providing a sample from the patient that may contain nucleic acids of prostate origin; and
(b) detecting the presence or absence in the sample of a gene fusion having a 5′ portion from a transcriptional regulatory region of an SLC45A3 gene and a 3′ portion from an ELK4 gene,
wherein detecting the presence in the sample of the gene fusion identifies prostate cancer in the patient.

2. The method of claim 1, wherein the transcriptional regulatory region of the SLC45A3 gene comprises a promoter region of the SLC45A3 gene.

3. The method of claim 1, wherein step (b) comprises detecting chimeric mRNA transcripts having a 5′ RNA portion transcribed from the transcriptional regulatory region of the SLC45A3 gene and a 3′ RNA portion transcribed from the ELK4 gene.

4. The method of claim 1, wherein said gene fusion is a read through transcript.

5. The method of claim 1, wherein the sample is selected from the group consisting of tissue, blood, plasma, serum, urine, urine supernatant, urine cell pellet, semen, prostatic secretions and prostate cells.

6. The method of claim 1, further comprising the step of detecting the presence or absence of a gene fusion having a 5′ portion from a transcriptional regulatory region of an androgen regultated gene or a housekeeping gene and a 3′ portion from an ETS family member gene.

7. A method for identifying prostate cancer in a patient comprising:
(a) providing a sample from the patient that may contain nucleic acids of prostate origin; and
(b) detecting the presence or absence in the sample of a gene fusion selected from the group consisting of USP10:ZDHHC7, EIF4E2:HJURP, HJURP:INPP4A, STRN4:GPSN2, RC3H2:RGS3, LMAN2:AP3S1, ZNF649-ZNF577 and MIPOL1:DGKB,
wherein detecting the presence in the sample of the gene fusion is identifies prostate cancer in the patient.

8. The method of claim 7, wherein step (b) comprises detecting chromosomal rearrangements of genomic DNA.

9. The method of claim 7, wherein step (b) comprises detecting chimeric mRNA transcripts.

10. The method of claim 7, wherein the sample is selected from the group consisting of tissue, blood, plasma, serum, urine, urine supernatant, urine cell pellet, semen, prostatic secretions and prostate cells.

11. A method for identifying prostate cancer in a patient comprising:
(a) providing a sample from the patient that may contain nucleic acids of prostate origin; and
(b) detecting the presence or absence in the sample of a gene fusion having a 5′ portion from a transcriptional regulatory region of an HERPUD1 gene and a 3′ portion from an ERG gene,
wherein detecting the presence in the sample of the gene fusion identifies prostate cancer in the patient.

12. A method for identifying prostate cancer in a patient comprising:
(a) providing a sample from the patient that may contain nucleic acids of prostate origin; and
(b) detecting the presence or absence in the sample of a gene fusion having a 5′ portion from a transcriptional regulatory region of an AX747630 gene and a 3′ portion from an ETV1 gene,
wherein detecting the presence in the sample of the gene fusion identifies prostate cancer in the patient.

13. A method for identifying prostate cancer in a patient comprising:
(a) providing a sample from the patient that may contain nucleic acids of prostate origin; and
(b) detecting the presence or absence in the sample of a gene fusion selected from the group consisting of TIA1:DIRC2, NUP214:XKR3, DLEU2:PSPC1, PIK3C2A:TEAD1, SPOCK1:TBC1D9B, and RERE:PIK3CD,
wherein detecting the presence in the sample of the gene fusion is identifies prostate cancer in the patient.

14. A method for identifying breast cancer in a patient comprising:
(a) providing a sample from the patient that may contain nucleic acids of breast origin; and
(b) detecting the presence or absence in the sample of a gene fusion selected from the group consisting of AHCYL1:RAD51C, ARHGAP19:DRG1, BC017255:TMEM49, FCHO1:MYO9B, and PAPOLA:AK7,
wherein detecting the presence in the sample of the gene fusion is identifies prostate cancer in the patient.

15. A method for identifying prostate cancer in a patient comprising:
(a) providing a sample from the patient that may contain nucleic acids of prostate origin; and
(b) detecting the presence or absence in the sample of a gene fusion selected from the group consisting of CARM1:YIPF2, MGC11102:BANF1, SLC4A1AP:SUPT7L, ERCC2:KLC3, PMF1:BGLAP, THOC6:HCFC1R1, NDUFB8:SEC31L2, ANKRD39:ANKRD23, C14orf124:KIAA0323, C14orf21:CIDEB, and ZNF511:TUBGCP2,
wherein detecting the presence in the sample of the gene fusion is identifies prostate cancer in the patient.

16. A composition comprising at least one of the following:
(a) an oligonucleotide probe comprising a sequence that hybridizes to a junction of a chimeric genomic DNA or chimeric mRNA in which a 5′ portion of the chimeric genomic DNA or chimeric mRNA is from a transcriptional regulatory region of an SLC45A3 gene and a 3′ portion of the chimeric genomic DNA or chimeric mRNA is from an ELK4 gene;
(b) a first oligonucleotide probe comprising a sequence that hybridizes to a 5′ portion of a chimeric genomic DNA or chimeric mRNA from a transcriptional regulatory region of an SLC45A3 gene and a second oligonucleotide probe comprising a sequence that hybridizes to a 3′ portion of the chimeric genomic DNA or chimeric mRNA from an ELK4 gene; and
(c) a first amplification oligonucleotide comprising a sequence that hybridizes to a 5′ portion of a chimeric genomic DNA or chimeric mRNA from a transcriptional regulatory region of an SLC45A3 gene and a second amplification oligonucleotide comprising a sequence that hybridizes to a 3′ portion of the chimeric genomic DNA or chimeric mRNA from an ERG gene.

17. A composition comprising at least one of the following:
(a) an oligonucleotide probe comprising a sequence that hybridizes to a junction of a chimeric genomic DNA or chimeric mRNA of a gene fusion selected from the group consisting of USP10:ZDHHC7, EIF4E2:HJURP, HJURP:INPP4A, STRN4:GPSN2, RC3H2:RGS3, LMAN2:AP3S1, ZNF649-ZNF577 and MIPOL1:DGKB;
(b) a first oligonucleotide probe comprising a sequence that hybridizes to a 5′ portion of a chimeric genomic DNA or chimeric mRNA from a gene fusion selected from the group consisting of USP10:ZDHHC7, EIF4E2:HJURP, HJURP:INPP4A, STRN4:GPSN2, RC3H2:RGS3, LMAN2:AP3S1, ZNF649-ZNF577 and MIPOL1:DGKB and a second oligonucleotide probe comprising a sequence that hybridizes to a 3′ portion of the chimeric genomic DNA or chimeric mRNA from a gene fusion selected from the group consisting of USP10:ZDHHC7, EIF4E2:HJURP, HJURP:INPP4A, STRN4:GPSN2, RC3H2:RGS3, LMAN2:AP3S1, ZNF649-ZNF577 and MIPOL1:DGKB;
(c) a first amplification oligonucleotide comprising a sequence that hybridizes to a 5′ portion of a chimeric genomic DNA or chimeric mRNA from a transcriptional regulatory region of an gene fusion selected from the group consisting of USP10:ZDHHC7, EIF4E2:HJURP, HJURP:INPP4A, STRN4:GPSN2, RC3H2:RGS3, LMAN2:AP3S1, ZNF649-ZNF577 and MIPOL1:DGKB and a second amplification oligonucleotide comprising a sequence that hybridizes to a 3′ portion of from a gene fusion selected from the group consisting of USP10:ZDHHC7, EIF4E2:HJURP, HJURP:INPP4A, STRN4:GPSN2, RC3H2:RGS3, LMAN2:AP3S1, ZNF649-ZNF577 and MIPOL1:DGKB.

18. A composition comprising at least one of the following:
(a) an oligonucleotide probe comprising a sequence that hybridizes to a junction of a chimeric genomic DNA or chimeric mRNA of a gene fusion selected from the group consisting of HERPUD1:ERG, AX747630:ETV1, TIA1:DIRC2, NUP214:XKR3, DLEU2:PSPC1, PIK3C2A:TEAD1, SPOCK1:TBC1D9B, RERE:PIK3CD, AHCYL1:RAD51C, ARHGAP19:DRG1, BC017255:TMEM49, FCHO1:MYO9B, PAPOLA:AK7, CARM1:YIPF2, MGC11102:BANF1, SLC4A1AP:SUPT7L, ERCC2:KLC3, PMF1:BGLAP, THOC6:HCFC1R1, NDUFB8:SEC31L2, ANKRD39:ANKRD23, C14orf124:KIAA0323, C14orf21:CIDEB, and ZNF511:TUBGCP2;
(b) a first oligonucleotide probe comprising a sequence that hybridizes to a 5′ portion of a chimeric genomic DNA or chimeric mRNA from a gene fusion selected from the group consisting of HERPUD1:ERG, AX747630:ETV1, TIA1:DIRC2, NUP214:XKR3, DLEU2:PSPC1, PIK3C2A:TEAD1, SPOCK1:TBC1D9B, RERE:PIK3CD, AHCYL1:RAD51C, ARHGAP19:DRG1, BC017255:TMEM49, FCHO1:MYO9B, PAPOLA:AK7, CARM1:YIPF2, MGC11102:BANF1, SLC4A1AP:SUPT7L, ERCC2:KLC3, PMF1:BGLAP, THOC6:HCFC1R1, NDUFB8:SEC31L2, ANKRD39:ANKRD23, C14orf124:KIAA0323, C14orf21:CIDEB, and ZNF511:TUBGCP2 and a second oligonucleotide probe comprising a sequence that hybridizes to a 3′ portion of the chimeric genomic DNA or chimeric mRNA from a gene fusion selected from the group consisting of HERPUD1:ERG, AX747630:ETV1, TIA1:DIRC2, NUP214:XKR3, DLEU2:PSPC1, PIK3C2A:TEAD1, SPOCK1:TBC1D9B, RERE:PIK3CD, AHCYL1:RAD51C, ARHGAP19:DRG1, BC017255:TMEM49, FCHO1:MYO9B, PAPOLA:AK7, CARM1:YIPF2, MGC11102:BANF1, SLC4A1AP:SUPT7L, ERCC2:KLC3, PMF1:BGLAP, THOC6:HCFC1R1, NDUFB8:SEC31L2, ANKRD39:ANKRD23, C14orf124:KIAA0323, C14orf21:CIDEB, and ZNF511:TUBGCP2;
(c) a first amplification oligonucleotide comprising a sequence that hybridizes to a 5′ portion of a chimeric genomic DNA or chimeric mRNA from a transcriptional regulatory region of an gene fusion selected from the group consisting of HERPUD1:ERG, AX747630:ETV1, TIA1:DIRC2, NUP214:XKR3, DLEU2:PSPC1, PIK3C2A:TEAD1, SPOCK1:TBC1D9B, RERE:PIK3CD, AHCYL1:RAD51C, ARHGAP19:DRG1, BC017255:TMEM49, FCHO1:MYO9B, and PAPOLA:AK7, CARM1:YIPF2, MGC11102:BANF1, SLC4A1AP:SUPT7L, ERCC2:KLC3, PMF1:BGLAP, THOC6:HCFC1R1, NDUFB8:SEC31L2, ANKRD39:ANKRD23, C14orf124:KIAA0323, C14orf21:CIDEB, ZNF511:TUBGCP2 and a second amplification oligonucleotide comprising a sequence that hybridizes to a 3′ portion of from a gene fusion selected from the group consisting of HERPUD1:ERG, AX747630:ETV1, TIA1:DIRC2, NUP214:XKR3, DLEU2:PSPC1, PIK3C2A:TEAD1, SPOCK1:TBC1D9B, RERE:PIK3CD, AHCYL1:RAD51C, ARHGAP19:DRG1, BC017255:TMEM49, FCHO1:MYO9B, PAPOLA:AK7, CARM1:YIPF2, MGC11102:BANF1, SLC4A1AP:SUPT7L, ERCC2:KLC3, PMF1:BGLAP, THOC6:HCFC1R1, NDUFB8:SEC31L2, ANKRD39:ANKRD23, C14orf124:KIAA0323, C14orf21:CIDEB, and ZNF511:TUBGCP2.
